SEQUENCE LISTING (121 sequences)

SEQ ID NO: 1 - protein, 7 aa, synthetic construct
RFQSNPM

SEQ ID NO: 2 - protein, 7 aa, synthetic construct
SNVVPVG

SEQ ID NO: 3 - protein, 7 aa, synthetic construct
MMHHDSH

SEQ ID NO: 4 - protein, 7 aa, synthetic construct
KVTAPHS

SEQ ID NO: 5 - protein, 7 aa, synthetic construct
AHAKGMD

SEQ ID NO: 6 - protein, 7 aa, synthetic construct
LHKNYGV

SEQ ID NO: 7 - protein, 7 aa, synthetic construct
VIGIKGA

SEQ ID NO: 8 - protein, 7 aa, synthetic construct
PHVMVPV

SEQ ID NO: 9 - protein, 7 aa, synthetic construct
MELGSFT

SEQ ID NO: 10 - protein, 7 aa, synthetic construct
MVPGKGD

SEQ ID NO: 11 - protein, 7 aa, synthetic construct
NTIRREN

SEQ ID NO: 12 - protein, 7 aa, synthetic construct
NGVKDRH

SEQ ID NO: 13 - protein, 7 aa, synthetic construct
NVDRPRA

SEQ ID NO: 14 - protein, 7 aa, synthetic construct
NDSRARM

SEQ ID NO: 15 - protein, 7 aa, synthetic construct
KAVTTDI

SEQ ID NO: 16 - protein, 7 aa, synthetic construct
IFPSTTK

SEQ ID NO: 17 - protein, 7 aa, synthetic construct
EHKAIMM

SEQ ID NO: 18 - protein, 7 aa, synthetic construct
LKDAVVK

SEQ ID NO: 19 - protein, 7 aa, synthetic construct
NNVMKHS

SEQ ID NO: 20 - protein, 7 aa, synthetic construct
NAVRMAP

SEQ ID NO: 21 - protein, 7 aa, synthetic construct
NATKVSW

SEQ ID NO: 22 - protein, 7 aa, synthetic construct
NSTRATM

SEQ ID NO: 23 - protein, 7 aa, synthetic construct
MNDTSRS

SEQ ID NO: 24 - protein, 7 aa, synthetic construct
NVVRPVA

SEQ ID NO: 25 - protein, 7 aa, synthetic construct
NQVRHFS

SEQ ID NO: 26 - protein, 7 aa, synthetic construct
KDSIERF

SEQ ID NO: 27 - protein, 7 aa, synthetic construct
NFRHHGL

SEQ ID NO: 28 - protein, 7 aa, synthetic construct
NEMNRSR

SEQ ID NO: 29 - protein, 7 aa, synthetic construct
NHDRGTN

SEQ ID NO: 30 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 31 - protein, 735 aa, synthetic construct [sequence omitted]

SEQ ID NO: 32 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 33 - protein, 736 aa, synthetic construct; identical to SEQ ID NO: 32

SEQ ID NO: 34 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPEQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 35 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 36 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 37 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 38 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 39 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 41 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 42 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 43 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 44 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 45 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 46 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQGQRQAAATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQGQSQAAATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 49 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 50 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQGQRQAATTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 51 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQGQSQAATTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 52 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQGQRGAATTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 53 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQGQSGAATTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 54 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPEQNGNTATVPGTLIFTSEEELAATNATDTDMWGNLPGGDQGQRQAATVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 55 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPEQNGNTATVPGTLIFTSEEELAATNATDTDMWGNLPGGDQGQSQAATVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 56 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQGQRQAAATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 57 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQGQSQAAATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 58 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQRQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 59 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQGQRQAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 61 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQGQSQAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 62 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQRQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 63 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQSQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 64 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQRQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 66 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQGQRQAAIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 67 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQGQSQAAIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 68 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQGQRQAAITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 69 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQGQSQAAITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 70 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQGQRQAAHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 71 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQGQSQAAHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 72 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNGQRQAATVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 73 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNGQSQAATVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 74 - protein, 737 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLGQRQAAIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 75 - protein, 737 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLGQSQAAIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 76 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQGQRQAAIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 77 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQGQSQAAIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 78 - protein, 5 aa, synthetic construct
GSGGS

SEQ ID NO: 79 - protein, 6 aa, synthetic construct
GGSGGS

SEQ ID NO: 80 - protein, 4 aa, synthetic construct
GGGS

SEQ ID NO: 81 - protein, 4 aa, synthetic construct
GGSG

SEQ ID NO: 82 - protein, 5 aa, synthetic construct
GGSGG

SEQ ID NO: 83 - protein, 5 aa, synthetic construct
GSGSG

SEQ ID NO: 84 - protein, 5 aa, synthetic construct
GSGGG

SEQ ID NO: 85 - protein, 5 aa, synthetic construct
GGGSG

SEQ ID NO: 86 - protein, 5 aa, synthetic construct
GSSSG

SEQ ID NO: 87 - protein, 42 aa, synthetic construct
PVATERFGTVAVNFQSSSTDPATGDVHAMGALPGMVWQDRDV

SEQ ID NO: 88 - protein, 42 aa, synthetic construct
PVATEQYGSVSTNLQRGNRQAATADVNTQGVLPGMVWQDRDV

SEQ ID NO: 89 - protein, 39 aa, synthetic construct
PVATEQYGTVANNLQSSNTAPTTRTVNDQGALPGMVWQD

SEQ ID NO: 90 - protein, 41 aa, synthetic construct
ATDTDMWGNLPGGDQSNSNLPTVDRLTALGAVPGMVWQNRD

SEQ ID NO: 91 - protein, 42 aa, synthetic construct
RVAYNVGGQMATNNQSSTTAPATGTYNLQEIVPGSVWMERDV

SEQ ID NO: 92 - protein, 42 aa, synthetic construct
PVATERFGTVAVNLQSSSTDPATGDVHVMGALPGMVWQDRDV

SEQ ID NO: 93 - protein, 42 aa, synthetic construct
PVATEEYGIVSSNLQAANTAAQTQVVNNQGALPGMVWQNRDV

SEQ ID NO: 94 - protein, 42 aa, synthetic construct
PVATEEYGIVADNLQQQNTAPQIGTVNSQGALPGMVWQNRDV

SEQ ID NO: 95 - protein, 42 aa, synthetic construct
PVATESYGQVATNHQSAQAQAQTGWVQNQGILPGMVWQDRDV

SEQ ID NO: 96 - protein, 42 aa, synthetic construct
PVATEQYGVVADNLQQANTGPIVGNVNSQGALPGMVWQNRDV

SEQ ID NO: 97 - protein, 42 aa, synthetic construct
PRDTDMFGQIADNNQNATTAPITGNVTAMGVLPGMVWQNRDI

SEQ ID NO: 98 - protein, 42 aa, synthetic construct
PRDTDMFGQIADNNQNATTAPHIANLDAMGIVPGMVWQNRDI

SEQ ID NO: 99 - protein, 42 aa, synthetic construct
PVATEQYGTVSNNLQNSNAGPTTGTVNHQGALPGMVWQDRDV

SEQ ID NO: 100 - protein, 42 aa, synthetic construct
PVATEQYGVVADNLQQQNAAPIVGAVNSQGALPGMVWQNRDV

SEQ ID NO: 101 - protein, 42 aa, synthetic construct; identical to SEQ ID NO: 100

SEQ ID NO: 102 - DNA, 45 bp, synthetic construct
  source 1..45: mol_type = other DNA
ggccagagwggcnnnnnnnnnnnnnnnnnnnnngcccaggcggcc

SEQ ID NO: 103 - protein, 15 aa, synthetic construct
GQSGXXXXXXXAQAA

SEQ ID NO: 104 - DNA, 45 bp, synthetic construct
  source 1..45: mol_type = other DNA
ggccagagwggcaacnnnnnncgtnnnnnnnnngcccaggcggcc

SEQ ID NO: 105 - protein, 15 aa, synthetic construct
GQSGNXXRXXXAQAA

SEQ ID NO: 106 - DNA, 20 bp, synthetic construct
  source 1..20: mol_type = other DNA
cgagacgctccttcctctca

SEQ ID NO: 107 - DNA, 24 bp, synthetic construct
  source 1..24: mol_type = other DNA
gtagatctctcgagcagcatctcg

SEQ ID NO: 108 - DNA, 65 bp, synthetic construct
  source 1..65: mol_type = other DNA
tctttccctacacgacgctcttccgatctnnnnnnnnnnnngtagatctctcgagcagcatctcg

SEQ ID NO: 109 - DNA, 54 bp, synthetic construct
  source 1..54: mol_type = other DNA
gtgactggagttcagacgtgtgctcttccgatctgccacctcccacctaggcta

SEQ ID NO: 110 - DNA, 59 bp, synthetic construct
  source 1..59: mol_type = other DNA
aatgatacggcgaccaccgagatctacacnnnnnnnnacactctttccctacacgacgc

SEQ ID NO: 111 - DNA, 50 bp, synthetic construct
  source 1..50: mol_type = other DNA
caagcagaagacggcatacgagatnnnnnnnngtgactggagttcagacg

SEQ ID NO: 112 - DNA, 29 bp, synthetic construct
  source 1..29: mol_type = other DNA
tctttccctacacgacgctcttccgatct

SEQ ID NO: 113 - DNA, 86 bp, synthetic construct
  source 1..86: mol_type = other DNA
caagcagaagacggcatacgagatagagtggagtgactggagttcagacgtgtgctcttccgatctgccacctcccacctaggcta

SEQ ID NO: 114 - DNA, 20 bp, synthetic construct
  source 1..20: mol_type = other DNA
gccacctcccacctaggcta

SEQ ID NO: 115 - DNA, 20 bp, synthetic construct
  source 1..20: mol_type = other DNA
aaagctgatgtgggaggagc

SEQ ID NO: 116 - DNA, 20 bp, synthetic construct
  source 1..20: mol_type = other DNA
ttcccgttctcagccttcac

SEQ ID NO: 117 - protein, 9 aa, synthetic construct
PERTAMSLP

SEQ ID NO: 118 - protein, 9 aa, synthetic construct
QQQNTAPQI

SEQ ID NO: 119 - protein, 9 aa, synthetic construct
LALGETTRP

SEQ ID NO: 120-121 - skipped (2 entries)

(19) United States
(12) Patent Application Publication
(10) Pub. No.: US 2026/0042803 A1
(43) Pub. Date: Feb. 12, 2026

(54) VARIANT AAV CAPSID POLYPEPTIDES TARGETING THE EYE

(71) Applicants: REVVITY GENE DELIVERY GMBH, Gräfelfing (DE); UNIVERSITÄT HEIDELBERG, Heidelberg (DE)

(72) Inventors: Christian Olivier THIRION, Munich (DE); Yvonne BERGHÖFER-HOCHHEIMER, Munich (DE); Dirk GRIMM, Heidelberg (DE); Kleopatra RAPTI, Heidelberg (DE)

(73) Assignees: REVVITY GENE DELIVERY GMBH, Gräfelfing (DE); UNIVERSITÄT HEIDELBERG, Heidelberg (DE)

(21) Appl. No.: 19/294,728

(22) Filed: Aug. 8, 2025

(30) Foreign Application Priority Data
Aug. 9, 2024 (EP) 24193921.4

(51) Int. Cl. C07K 14/005; A61K 48/00; C12N 15/86

(52) U.S. Cl. CPC C07K 14/005; A61K 48/0075; C12N 15/86; C12N 2750/14122; C12N 2750/14143

(57) ABSTRACT

The present application relates to (i) a variant adeno-associated virus (AAV) capsid polypeptide comprising a peptide insertion in the variable region IV or in the variable region VIII relative to a wild-type AAV capsid polypeptide, wherein the peptide insertion comprises an amino acid sequence selected from the group consisting of SEQ ID NOs: 1-29 or an amino acid sequence having at least 70% sequence identity thereto, (ii) an isolated nucleic acid encoding the aforementioned variant polypeptide, (iii) a recombinant polynucleotide comprising the aforementioned nucleic acid, and (iv) an isolated cell comprising the aforementioned polypeptide, nucleic acid or recombinant polynucleotide. The present application further relates to (v) an adeno-associated virus (AAV) vector comprising the aforementioned variant polypeptide, (vi) a pharmaceutical composition comprising the aforementioned AAV vector as well as (vii) the use of the aforementioned vector or pharmaceutical composition in preventing or treating an ocular disease. Finally, the present application relates to (viii) a method of delivering a heterologous nucleic acid to a retinal cell and (ix) a method of delivering a heterologous nucleic acid to the eye of a subject.

### REFERENCE TO RELATED APPLICATIONS

This application claims priority to European Application 24193921.4, filed Aug. 9, 2024, the entire content of which is incorporated herein by reference.

### SEQUENCE LISTING

The instant application contains a Sequence Listing which has been submitted electronically in XML format and is hereby incorporated by reference in its entirety. Said XML format Sequence Listing, created on Aug. 6, 2025, is named R10865WO.xml and is 140,081 bytes in size.

### TECHNICAL FIELD

The present application is in the field of AAV vectors targeting the eye. In particular, the present application is concerned with a variant adeno-associated virus (AAV) capsid polypeptide comprising a peptide insertion in the variable region IV or the variable region VIII relative to a wild-type AAV capsid polypeptide, wherein the peptide insertion comprises an amino acid sequence selected from the group consisting of SEQ ID NOs: 1-29 or an amino acid sequence having at least 70% sequence identity thereto, and an isolated nucleic acid encoding the same. Further, the present application relates to a recombinant polynucleotide comprising the isolated nucleic acid and an isolated cell comprising the polypeptide, nucleic acid or recombinant polynucleotide of the present disclosure. Still further, the present application is related to an adeno-associated virus (AAV) vector comprising the variant polypeptide of the disclosure, a pharmaceutical composition comprising the same as well as the use of the vector or pharmaceutical composition in preventing or treating an ocular disease. Finally, the present application is concerned with a method of delivering a heterologous nucleic acid to a retinal cell and a method of delivering a heterologous nucleic acid to the eye of a subject.

### BACKGROUND OF THE DISCLOSURE

Adeno-associated viruses (AAVs) belong to the Parvoviridae family. Vectors derived from AAV are attractive for delivering genetic material because they are able to transduce a wide variety of non-dividing and dividing cell types, they are devoid of the virus replication, assembly and structural genes, thereby reducing the host cell responses to virus infection (such as e.g., interferon-mediated responses), and, because they can be employed as replication-deficient AAV vectors, there is a limited risk of insertional mutagenesis or genotoxicity. Further, AAVs have not been reported to cause any diseases. At present, there are eight AAV-based gene delivery medications approved by the FDA and/or the EMA, namely Glybera (withdrawn due to commercialization challenges), Luxturna, Zolgensma, Upstaza, Roctavian, Hemgenix, Elevidys and Beqvez (see for some of these also Table 1 of He X et al. Research. 2023; 6; Article 0291).

Introduction and expression of a heterologous nucleic acid in the eye has great therapeutic value in treating a variety of ophthalmic diseases that arise from or are related to eye cells or eye function. Thus, the main goal in ophthalmic gene therapy is the targeted delivery of a heterologous nucleic acid, such as e.g., a therapeutic gene, an interfering RNA or a gene editing tool, to eye cells, in particular to the neuroretina, the retinal pigment epithelium (RPE) or even to distinct cell populations of the aforementioned cells, especially in the context of degenerated cell layers, a characteristic of a progressed retinal disease. The first approved eye gene therapy is Luxturna (the tradename for the gene therapy referred to as voretigene neparvovec), which is based on AAV2 and carries a gene copy of RPE65, an enzyme of the visual cycle located in the RPE. Furthermore, many AAV-based gene therapies are presently undergoing clinical trials for ocular diseases, see e.g., Table 2 of He X et al., supra.

However, despite the existence of many AAV-based gene therapies, there is nevertheless still the need to provide further, in particular improved AAV vectors with high specificity to the eye and/or efficient eye transduction for successful eye-targeted gene therapy.

### SUMMARY OF THE DISCLOSURE

The present disclosure solves the above need in that it provides a variant AAV capsid polypeptide that comprises a specific peptide insertion, which results in an improved targeting to the eye (including specific cells thereof) and/or an improved transduction of (specific) cells of the eye.

In a first aspect, the present disclosure is directed to a variant adeno-associated virus (AAV) capsid polypeptide comprising a peptide insertion in the variable region IV or in the variable region VIII relative to a wild-type AAV capsid polypeptide, wherein the peptide insertion comprises an amino acid sequence selected from the group consisting of SEQ ID NOs: 1-29 or an amino acid sequence having at least 70%, preferably at least 85% sequence identity thereto.

### Embodiments Relating to the Peptide Insertion

As noted above in the first aspect, the peptide insertion comprises an amino acid sequence selected from the group consisting of SEQ ID NOs: 1-29 or an amino acid sequence having at least 70% sequence identity thereto. Any of the amino acid sequences of SEQ ID NOs: 1-29 is seven amino acids long. An amino acid sequence having at least 70% sequence identity thereto means that the sequence has at least five amino acids in common with the seven amino acids as defined in any of SEQ ID NOs: 1-29.

As also noted above in the first aspect, the peptide insertion preferably comprises an amino acid sequence selected from the group consisting of SEQ ID NOs: 1-29 or an amino acid sequence having at least 85% sequence identity thereto. An amino acid sequence having at least 85% sequence identity thereto means that the sequence has at least six amino acids in common with the seven amino acids as defined in any of SEQ ID NOs: 1-29. In one embodiment of the first aspect, the peptide insertion comprises an amino acid sequence selected from the group consisting of SEQ ID NOs: 1-29 or an amino acid sequence having at least 85% sequence identity thereto.

In one embodiment of the first aspect, the peptide insertion comprises an amino acid sequence selected from the group consisting of SEQ ID NOs: 1-29.

In one embodiment of the first aspect, the peptide insertion comprises an amino acid sequence selected from the group consisting of SEQ ID NOs: 1-10 or an amino acid sequence having at least 70% sequence identity thereto. In one embodiment of the first aspect, the peptide insertion comprises an amino acid sequence selected from the group consisting of SEQ ID NOs: 1-10 or an amino acid sequence having at least 85% sequence identity thereto. In one embodiment of the first aspect, the peptide insertion comprises an amino acid sequence selected from the group consisting of SEQ ID NOs: 1-10.

In one embodiment of the first aspect, the peptide insertion comprises the amino acid sequence of SEQ ID NO:1 or an amino acid sequence having at least 70% sequence identity thereto, or SEQ ID NO: 2 or an amino acid sequence having at least 70% sequence identity thereto, or SEQ ID NO:3 or an amino acid sequence having at least 70% sequence identity thereto, or SEQ ID NO:4 or an amino acid sequence having at least 70% sequence identity thereto, or SEQ ID NO:5 or an amino acid sequence having at least 70% sequence identity thereto, or SEQ ID NO:6 or an amino acid sequence having at least 70% sequence identity thereto, or SEQ ID NO:7 or an amino acid sequence having at least 70% sequence identity thereto. In one embodiment of the first aspect, the peptide insertion comprises the amino acid sequence of SEQ ID NO:1 or an amino acid sequence having at least 85% sequence identity thereto, or SEQ ID NO:2 or an amino acid sequence having at least 85% sequence identity thereto, or SEQ ID NO:3 or an amino acid sequence having at least 85% sequence identity thereto, or SEQ ID NO:4 or an amino acid sequence having at least 85% sequence identity thereto, or SEQ ID NO:5 or an amino acid sequence having at least 85% sequence identity thereto, or SEQ ID NO: 6 or an amino acid sequence having at least 85% sequence identity thereto, or SEQ ID NO:7 or an amino acid sequence having at least 85% sequence identity thereto. In one embodiment of the first aspect, the peptide insertion comprises the amino acid sequence of SEQ ID NO:1 or SEQ ID NO: 2 or SEQ ID NO:3 or SEQ ID NO:4 or SEQ ID NO:5 or SEQ ID NO:6 or SEQ ID NO:7.

In one embodiment of the first aspect, the peptide insertion comprises the amino acid sequence of SEQ ID NO:1 or an amino acid sequence having at least 70% sequence identity thereto, or SEQ ID NO: 2 or an amino acid sequence having at least 70% sequence identity thereto. In one embodiment of the first aspect, the peptide insertion comprises the amino acid sequence of SEQ ID NO:1 or an amino acid sequence having at least 85% sequence identity thereto, or SEQ ID NO:2 or an amino acid sequence having at least 85% sequence identity thereto. In one embodiment of the first aspect, the peptide insertion comprises the amino acid sequence of SEQ ID NO:1 or SEQ ID NO:2.

In one embodiment of the first aspect, the peptide insertion comprises the amino acid sequence of SEQ ID NO:2 or an amino acid sequence having at least 70% sequence identity thereto, or SEQ ID NO: 10 or an amino acid sequence having at least 70% sequence identity thereto. In one embodiment of the first aspect, the peptide insertion comprises the amino acid sequence of SEQ ID NO:2 or an amino acid sequence having at least 85% sequence identity thereto, or SEQ ID NO:10 or an amino acid sequence having at least 85% sequence identity thereto. In one embodiment of the first aspect, the peptide insertion comprises the amino acid sequence of SEQ ID NO:2 or SEQ ID NO:10.

In a first alternative relating to all of the aforementioned embodiments, the peptide insertion consists of the sequences as defined therein. The following embodiments relate to this first alternative.

In one embodiment of the first aspect, the peptide insertion consists of an amino acid sequence selected from the group consisting of SEQ ID NOs: 1-29 or an amino acid sequence having at least 70% sequence identity thereto. In one embodiment of the first aspect, the peptide insertion consists of an amino acid sequence selected from the group consisting of SEQ ID NOs: 1-29 or an amino acid sequence having at least 85% sequence identity thereto. In one embodiment of the first aspect, the peptide insertion consists of an amino acid sequence selected from the group consisting of SEQ ID NOs: 1-29.

In one embodiment of the first aspect, the peptide insertion consists of an amino acid sequence selected from the group consisting of SEQ ID NOs: 1-10 or an amino acid sequence having at least 70% sequence identity thereto. In one embodiment of the first aspect, the peptide insertion consists of an amino acid sequence selected from the group consisting of SEQ ID NOs: 1-10 or an amino acid sequence having at least 85% sequence identity thereto. In one embodiment of the first aspect, the peptide insertion consists of an amino acid sequence selected from the group consisting of SEQ ID NOs: 1-10.

In one embodiment of the first aspect, the peptide insertion consists of the amino acid sequence of SEQ ID NO:1 or an amino acid sequence having at least 70% sequence identity thereto, or SEQ ID NO: 2 or an amino acid sequence having at least 70% sequence identity thereto, or SEQ ID NO:3 or an amino acid sequence having at least 70% sequence identity thereto, or SEQ ID NO:4 or an amino acid sequence having at least 70% sequence identity thereto, or SEQ ID NO:5 or an amino acid sequence having at least 70% sequence identity thereto, or SEQ ID NO:6 or an amino acid sequence having at least 70% sequence identity thereto, or SEQ ID NO:7 or an amino acid sequence having at least 70% sequence identity thereto. In one embodiment of the first aspect, the peptide insertion consists of the amino acid sequence of SEQ ID NO:1 or an amino acid sequence having at least 85% sequence identity thereto, or SEQ ID NO:2 or an amino acid sequence having at least 85% sequence identity thereto, or SEQ ID NO:3 or an amino acid sequence having at least 85% sequence identity thereto, or SEQ ID NO:4 or an amino acid sequence having at least 85% sequence identity thereto, or SEQ ID NO:5 or an amino acid sequence having at least 85% sequence identity thereto, or SEQ ID NO: 6 or an amino acid sequence having at least 85% sequence identity thereto, or SEQ ID NO:7 or an amino acid sequence having at least 85% sequence identity thereto. In one embodiment of the first aspect, the peptide insertion consists of the amino acid sequence of SEQ ID NO:1 or SEQ ID NO: 2 or SEQ ID NO:3 or SEQ ID NO:4 or SEQ ID NO:5 or SEQ ID NO:6 or SEQ ID NO:7.

In one embodiment of the first aspect, the peptide insertion consists of the amino acid sequence of SEQ ID NO:1 or an amino acid sequence having at least 70% sequence identity thereto, or SEQ ID NO: 2 or an amino acid sequence having at least 70% sequence identity thereto. In one embodiment of the first aspect, the peptide insertion consists of the amino acid sequence of SEQ ID NO:1 or an amino acid sequence having at least 85% sequence identity thereto, or SEQ ID NO:2 or an amino acid sequence having at least 85% sequence identity thereto. In one embodiment of the first aspect, the peptide insertion consists of the amino acid sequence of SEQ ID NO:1 or SEQ ID NO:2.

In one embodiment of the first aspect, the peptide insertion consists of the amino acid sequence of SEQ ID NO:2 or an amino acid sequence having at least 70% sequence identity thereto, or SEQ ID NO: 10 or an amino acid sequence having at least 70% sequence identity thereto. In one embodiment of the first aspect, the peptide insertion consists of the amino acid sequence of SEQ ID NO:2 or an amino acid sequence having at least 85% sequence identity thereto, or SEQ ID NO:10 or an amino acid sequence having at least 85% sequence identity thereto. In one embodiment of the first aspect, the peptide insertion consists of the amino acid sequence of SEQ ID NO:2 or SEQ ID NO:10.

In a second alternative, the peptide insertion comprises at least one further amino acid in addition to the sequences as defined herein. The following embodiments relate to this second alternative.

In some embodiments, any of the sequences as defined in SEQ ID NOs: 1-29 comprises at the N-terminus about 10, about 9, about 8, about 7, about 6, about 5, about 4, about 3, about 2, or about 1 amino acids in addition to the respective sequence set out in SEQ ID NOs: 1-29 and/or any of the sequences as defined in SEQ ID NOs: 1-29 comprises at the C-terminus about 10, about 9, about 8, about 7, about 6, about 5, about 4, about 3, about 2, or about 1 amino acids in addition to the respective sequence set out in SEQ ID NOs: 1-29. In other embodiments, any of the sequences as defined in SEQ ID NOs: 1-29 comprises at the N-terminus one or two amino acids in addition to the respective sequence set out in SEQ ID NOs: 1-29 and at the C-terminus one or two amino acids in addition to the respective sequence set out in SEQ ID NOs: 1-29.

In some embodiments, any of the sequences as defined in SEQ ID NOs: 1-29 comprises at the N-terminus a linker or a flexible linker of about 10, about 9, about 8, about 7, about 6, about 5, about 4, about 3, about 2, or about 1 amino acids, in addition to the respective sequence set out in SEQ ID NOs: 1-29 and/or any of the sequences as defined in SEQ ID NOs: 1-29 comprises at the C-terminus a linker or a flexible linker of about 10, about 9, about 8, about 7, about 6, about 5, about 4, about 3, about 2, or about 1 amino acids, in addition to the respective sequence set out in SEQ ID NOs: 1-29. A flexible linker can in particular be comprised of small amino acids, such as glycine and alanine. The creation of such linker sequences is routine to those of skill in the art. Examples of linker sequences include proline polymers (P)n, glycine polymers (G)n, glycine-serine polymers [including, for example, (GS)n, GSGGS (SEQ ID NO:78), (GGSGGS)n (SEQ ID NO:79), and (GGGS)n (SEQ ID NO:80), where n is an integer of at least one], glycine-alanine polymers and alanine-serine polymers. Exemplary linkers can comprise amino acid sequences including, but not limited to, GGSG (SEQ ID NO: 81), GGSGG (SEQ ID NO:82), GSGSG (SEQ ID NO:83), GSGGG (SEQ ID NO:84), GGGSG (SEQ ID NO:85), GSSSG (SEQ ID NO:86), and the like.

In some embodiments, any of the sequences as defined in SEQ ID NOs: 1-29 further comprises one amino acid at the N-terminus and/or one amino acid at the C-terminus. In some embodiments, any of the sequences as defined in SEQ ID NOs: 1-29 further comprises a glycine (G) at the N-terminus and/or an alanine (A) at the C-terminus. The sequence identities given in the next paragraphs apply to the sequence including the G at the N-terminus and/or the A at the C-terminus.

In the next paragraphs, the sequence is defined by a G at the N-terminus, followed by an amino acid sequence selected from the group consisting of SEQ ID NOs: 1-29, so overall eight amino acids. The sequence identity as given in the next paragraphs applies to the overall eight amino acids.

In one embodiment of the first aspect, the peptide insertion comprises an amino acid sequence of a G at the N-terminus, followed by an amino acid sequence selected from the group consisting of SEQ ID NOs: 1-29, or an amino acid sequence having at least 75% sequence identity thereto. Any of the aforementioned sequences is eight amino acids long. An amino acid sequence having at least 75% sequence identity thereto means that the sequence has at least six amino acids in common with these eight amino acids. In one embodiment of the first aspect, the peptide insertion comprises an amino acid sequence of a G at the N-terminus, followed by an amino acid sequence selected from the group consisting of SEQ ID NOs: 1-29, or an amino acid sequence having at least 87% sequence identity thereto. Any of the aforementioned sequences is eight amino acids long. An amino acid sequence having at least 87% sequence identity thereto means that the sequence has at least seven amino acids in common with these eight amino acids. In one embodiment of the first aspect, the peptide insertion comprises an amino acid sequence of a G at the N-terminus, followed by an amino acid sequence selected from the group consisting of SEQ ID NOs: 1-29.

In one embodiment of the first aspect, the peptide insertion comprises an amino acid sequence of a G at the N-terminus, followed by an amino acid sequence selected from the group consisting of SEQ ID NOs: 1-10, or an amino acid sequence having at least 75% sequence identity thereto. In one embodiment of the first aspect, the peptide insertion comprises an amino acid sequence of a G at the N-terminus, followed by an amino acid sequence selected from the group consisting of SEQ ID NOs: 1-10, or an amino acid sequence having at least 87% sequence identity thereto. In one embodiment of the first aspect, the peptide insertion comprises an amino acid sequence of a G at the N-terminus, followed by an amino acid sequence selected from the group consisting of SEQ ID NOs: 1-10.

In one embodiment of the first aspect, the peptide insertion comprises an amino acid sequence of a G at the N-terminus, followed by the amino acid sequence of SEQ ID NO:1 or SEQ ID NO:2 or SEQ ID NO: 3 or SEQ ID NO:4 or SEQ ID NO:5 or SEQ ID NO:6 or SEQ ID NO:7, or an amino acid sequence having at least 75% sequence identity thereto. In one embodiment of the first aspect, the peptide insertion comprises an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO: 6 or SEQ ID NO:7, or an amino acid sequence having at least 87% sequence identity thereto. In one embodiment of the first aspect, the peptide insertion comprises an amino acid sequence of a G at the N-terminus, followed by the amino acid sequence of SEQ ID NO:1 or SEQ ID NO:2 or SEQ ID NO: 3 or SEQ ID NO:4 or SEQ ID NO:5 or SEQ ID NO:6 or SEQ ID NO:7.

In one embodiment of the first aspect, the peptide insertion comprises an amino acid sequence of a G at the N-terminus, followed by the amino acid sequence of SEQ ID NO:1 or SEQ ID NO:2, or an amino acid sequence having at least 75% sequence identity thereto. In one embodiment of the first aspect, the peptide insertion comprises an amino acid sequence of a G at the N-terminus, followed by the amino acid sequence of SEQ ID NO:1 or SEQ ID NO:2, or an amino acid sequence having at least 87% sequence identity thereto. In one embodiment of the first aspect, the peptide insertion comprises an amino acid sequence of a G at the N-terminus, followed by the amino acid sequence of SEQ ID NO: 1 or SEQ ID NO:2.

In one embodiment of the first aspect, the peptide insertion comprises an amino acid sequence of a G at the N-terminus, followed by the amino acid sequence of SEQ ID NO:2 or SEQ ID NO:10, or an amino acid sequence having at least 75% sequence identity thereto. In one embodiment of the first aspect, the peptide insertion comprises an amino acid sequence of a G at the N-terminus, followed by the amino acid sequence of SEQ ID NO:2 or SEQ ID NO:10, or an amino acid sequence having at least 87% sequence identity thereto. In one embodiment of the first aspect, the peptide insertion comprises an amino acid sequence of a G at the N-terminus, followed by the amino acid sequence of SEQ ID NO:2 or SEQ ID NO:10.

In the next paragraphs, the sequence is defined by a G at the N-terminus, followed by an amino acid sequence selected from the group consisting of SEQ ID NOs: 1-29, followed by an A at the C-terminus, so overall nine amino acids. The sequence identity as given in the next paragraphs applies to the overall nine amino acids.

In one embodiment of the first aspect, the peptide insertion comprises an amino acid sequence of a G at the N-terminus, followed by an amino acid sequence selected from the group consisting of SEQ ID NOs: 1-29, followed by an A at the C-terminus, or an amino acid sequence having at least 77% sequence identity thereto. Any of the aforementioned sequences is nine amino acids long. An amino acid sequence having at least 77% sequence identity thereto means that the sequence has at least seven amino acids in common with these nine amino acids. In one embodiment of the first aspect, the peptide insertion comprises an amino acid sequence of a G at the N-terminus, followed by an amino acid sequence selected from the group consisting of SEQ ID NOs: 1-29, followed by an A at the C-terminus, or an amino acid sequence having at least 88% sequence identity thereto. Any of the aforementioned sequences is nine amino acids long. An amino acid sequence having at least 88% sequence identity thereto means that the sequence has at least eight amino acids in common with these nine amino acids. In one embodiment of the first aspect, the peptide insertion comprises an amino acid sequence of a G at the N-terminus, followed by an amino acid sequence selected from the group consisting of SEQ ID NOs: 1-29, followed by an A at the C-terminus.

In one embodiment of the first aspect, the peptide insertion comprises an amino acid sequence of a G at the N-terminus, followed by an amino acid sequence selected from the group consisting of SEQ ID NOs: 1-10, followed by an A at the C-terminus, or an amino acid sequence having at least 77% sequence identity thereto. In one embodiment of the first aspect, the peptide insertion comprises an amino acid sequence of a G at the N-terminus, followed by an amino acid sequence selected from the group consisting of SEQ ID NOs: 1-10, followed by an A at the C-terminus, or an amino acid sequence having at least 88% sequence identity thereto. In one embodiment of the first aspect, the peptide insertion comprises an amino acid sequence of a G at the N-terminus, followed by an amino acid sequence selected from the group consisting of SEQ ID NOs: 1-10, followed by an A at the C-terminus.

In one embodiment of the first aspect, the peptide insertion comprises an amino acid sequence of a G at the N-terminus, followed by the amino acid sequence of SEQ ID NO:1 or SEQ ID NO:2 or SEQ ID NO: 3 or SEQ ID NO:4 or SEQ ID NO:5 or SEQ ID NO:6 or SEQ ID NO:7, followed by an A at the C-terminus, or an amino acid sequence having at least 77% sequence identity thereto. In one embodiment of the first aspect, the peptide insertion comprises an amino acid sequence of a G at the N-terminus, followed by the amino acid sequence of SEQ ID NO:1 or SEQ ID NO:2 or SEQ ID NO:3 or SEQ ID NO: 4 or SEQ ID NO:5 or SEQ ID NO:6 or SEQ ID NO:7, followed by an A at the C-terminus, or an amino acid sequence having at least 88% sequence identity thereto. In one embodiment of the first aspect, the peptide insertion comprises an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, followed by an A at the C-terminus.

In one embodiment of the first aspect, the peptide insertion comprises an amino acid sequence of a G at the N-terminus, followed by the amino acid sequence of SEQ ID NO:1 or SEQ ID NO:2, followed by an A at the C-terminus, or an amino acid sequence having at least 77% sequence identity thereto. In one embodiment of the first aspect, the peptide insertion comprises an amino acid sequence of a G at the N-terminus, followed by the amino acid sequence of SEQ ID NO:1 or SEQ ID NO:2, followed by an A at the C-terminus, or an amino acid sequence having at least 88% sequence identity thereto. In one embodiment of the first aspect, the peptide insertion comprises an amino acid sequence of a G at the N-terminus, followed by the amino acid sequence of SEQ ID NO:1 or SEQ ID NO:2, followed by an A at the C-terminus.

In one embodiment of the first aspect, the peptide insertion comprises an amino acid sequence of a G at the N-terminus, followed by the amino acid sequence of SEQ ID NO:2 or SEQ ID NO:10, followed by an A at the C-terminus, or an amino acid sequence having at least 77% sequence identity thereto. In one embodiment of the first aspect, the peptide insertion comprises an amino acid sequence of a G at the N-terminus, followed by the amino acid sequence of SEQ ID NO:2 or SEQ ID NO:10, followed by an A at the C-terminus, or an amino acid sequence having at least 88% sequence identity thereto. In one embodiment of the first aspect, the peptide insertion comprises an amino acid sequence of a G at the N-terminus, followed by the amino acid sequence of SEQ ID NO:2 or SEQ ID NO:10, followed by an A at the C-terminus.

In some of the above embodiments relating to the second alternative, the peptide insertion consists of the sequences as defined therein, as it is disclosed in the following embodiments. In one embodiment of the first aspect, the peptide insertion consists of an amino acid sequence of a G at the N-terminus, followed by an amino acid sequence selected from the group consisting of SEQ ID NOs: 1-29, followed by an A at the C-terminus, or an amino acid sequence having at least 77% sequence identity thereto. In one preferred embodiment of the first aspect, the peptide insertion consists of an amino acid sequence of a G at the N-terminus, followed by an amino acid sequence selected from the group consisting of SEQ ID NOs: 1-29, followed by an A at the C-terminus, or an amino acid sequence having at least 88% sequence identity thereto. In one preferred embodiment of the first aspect, the peptide insertion consists of an amino acid sequence of a G at the N-terminus, followed by an amino acid sequence selected from the group consisting of SEQ ID NOs: 1-29, followed by an A at the C-terminus.

In one embodiment of the first aspect, the peptide insertion consists of an amino acid sequence of a G at the N-terminus, followed by an amino acid sequence selected from the group consisting of SEQ ID NOs: 1-10, followed by an A at the C-terminus, or an amino acid sequence having at least 77% sequence identity thereto. In one preferred embodiment of the first aspect, the peptide insertion consists of an amino acid sequence of a G at the N-terminus, followed by an amino acid sequence selected from the group consisting of SEQ ID NOs: 1-10, followed by an A at the C-terminus, or an amino acid sequence having at least 88% sequence identity thereto. In one preferred embodiment of the first aspect, the peptide insertion consists of an amino acid sequence of a G at the N-terminus, followed by an amino acid sequence selected from the group consisting of SEQ ID NOs: 1-10, followed by an A at the C-terminus.

In one embodiment of the first aspect, the peptide insertion consists of an amino acid sequence of a G at the N-terminus, followed by the amino acid sequence of SEQ ID NO:1 or SEQ ID NO:2 or SEQ ID NO: 3 or SEQ ID NO:4 or SEQ ID NO:5 or SEQ ID NO:6 or SEQ ID NO:7, followed by an A at the C-terminus, or an amino acid sequence having at least 77% sequence identity thereto. In one preferred embodiment of the first aspect, the peptide insertion consists of an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, followed by an A at the C-terminus, or an amino acid sequence having at least 88% sequence identity thereto. In one preferred embodiment of the first aspect, the peptide insertion consists of an amino acid sequence of a G at the N-terminus, followed by the amino acid sequence of SEQ ID NO:1 or SEQ ID NO:2 or SEQ ID NO:3 or SEQ ID NO: 4 or SEQ ID NO:5 or SEQ ID NO:6 or SEQ ID NO:7, followed by an A at the C-terminus.

In one embodiment of the first aspect, the peptide insertion consists of an amino acid sequence of a G at the N-terminus, followed by the amino acid sequence of SEQ ID NO:1 or SEQ ID NO:2, followed by an A at the C-terminus, or an amino acid sequence having at least 77% sequence identity thereto. In one preferred embodiment of the first aspect, the peptide insertion consists of an amino acid sequence of a G at the N-terminus, followed by the amino acid sequence of SEQ ID NO:1 or SEQ ID NO: 2, followed by an A at the C-terminus, or an amino acid sequence having at least 88% sequence identity thereto. In one preferred embodiment of the first aspect, the peptide insertion consists of an amino acid sequence of a G at the N-terminus, followed by the amino acid sequence of SEQ ID NO:1 or SEQ ID NO:2, followed by an A at the C-terminus.

In one embodiment of the first aspect, the peptide insertion consists of an amino acid sequence of a G at the N-terminus, followed by the amino acid sequence of SEQ ID NO:2 or SEQ ID NO:10, followed by an A at the C-terminus, or an amino acid sequence having at least 77% sequence identity thereto. In one preferred embodiment of the first aspect, the peptide insertion consists of an amino acid sequence of a G at the N-terminus, followed by the amino acid sequence of SEQ ID NO:2 or SEQ ID NO: 10, followed by an A at the C-terminus, or an amino acid sequence having at least 88% sequence identity thereto. In one preferred embodiment of the first aspect, the peptide insertion consists of an amino acid sequence of a G at the N-terminus, followed by the amino acid sequence of SEQ ID NO:2 or SEQ ID NO: 10, followed by an A at the C-terminus.

### Embodiments Relating to the Capsid Polypeptide Excluding the Peptide Insertion

In one embodiment of the first aspect, the variable region IV (and thus the insertion site) is located in the stretch of amino acids comprised in the β-turn connecting the two β-sheets that build up the highest peak at the three-fold symmetry axis of the AAV capsid polypeptide. More particularly, the insertion site is located in the amino acids fragment connecting β-sheets GH2 and GH3. See, e.g., Boucas et al. J Gene Med 2009; 11:1103-1113, Xie et al. Proc Natl Acad Sci USA 2002; 99 (16): 10405-10 and Meyer et al. eLife 2019; 8: e44707. In some embodiments, the insertion site is within amino acids 446 to 470 of AAV1, within amino acids 445 to 469 or more specifically G451 to T455 of AAV2, within amino acids 445 to 470 of AAV3, within amino acids 445 to 470 of AAV3b, within amino acids 439 to 464 or more specifically G446 to A451 of AAV4, within amino acids 438 to 456 of AAV5, within amino acids 446 to 470 of AAV6, within amino acids 447 to 472 of AAV7, within amino acids 448 to 472 of AAV8, within amino acids 447 to 470 of AAV9, within amino acids 448 to 472 of AAV10, within amino acids 438 to 463 of AAV11, within amino acids 447 to 472 of AAV12, within amino acids 444 to 467 of AAV13, within amino acids 448 to 472 of AAVrh10, or within amino acids 448 to 472 of AAVrh74.

In one embodiment of the first aspect, the variable region VIII (and thus the insertion site) is located in the stretch of amino acids between strands BG and BH of the AAV capsid polypeptide. In some embodiments, the insertion site is within amino acids 580 to 595 of AAV1, within amino acids 579 to 594 of AAV2, within amino acids 580 to 595 of AAV3, within amino acids 580 to 595 of AAV3b, within amino acids 578 to 593 of AAV4, within amino acids 569 to 584 of AAV5, within amino acids 580 to 595 of AAV6, within amino acids 581 to 596 of AAV7, within amino acids 582 to 597 of AAV8, within amino acids 580 to 595 of AAV9, within amino acids 582 to 597 of AAV10, within amino acids 575 to 593 of AAV11, within amino acids 584 to 600 of AAV12, within amino acids 575 to 593 of AAV13, within amino acids 582 to 597 of AAVrh10, or within amino acids 582 to 597 of AAVrh74.

In one embodiment of the first aspect, the capsid polypeptide excluding the peptide insertion is a wild-type AAV capsid polypeptide or is derived from a wild-type AAV capsid polypeptide while retaining the ability to form a capsid. A capsid polypeptide excluding the peptide insertion that is derived from a wild-type AAV capsid can in particular comprise at least one modification (compared to a wild-type capsid polypeptide) that provides for a desired characteristic of the resulting capsid polypeptide independent from targeting to the eye. Examples of modifications in the wild-type capsid that provide desired characteristics include e.g., a decreased proteasomal degradation of the viral particle once internalized. The decreased degradation can be achieved, for example, by changing tyrosines, which can be phosphorylated and, therefore, target the capsid for degradation. The tyrosines can be changed to phenylalanines [in AAV2: Y252, 272, 444, 500, 700, 730F]. See e.g., Silva et al. Mol Ther. 2009; 17 (3): 463-71. An alternative way of decreasing degradation can be achieved by changing lysines to glutamic acid, see e.g., Büning and Srivastava, Mol Ther Methods Clin Dev. 2019:12:248-265.

In one embodiment of the first aspect, the variant AAV capsid polypeptide is selected from the group consisting of a VP1 polypeptide, a VP2 polypeptide, a VP3 polypeptide, and functional fragments thereof.

In one embodiment of the first aspect, the AAV is selected from the group consisting of AAV1, AAV2, AAV3, AAV3b, AAV4, AAV5, AAV6, AAV7, AAV8, AAV9, AAV10, AAV11, AAV12, AAV13, AAVrh10 and AAVrh74. In another embodiment, the AAV is selected from the group consisting of AAV2, AAV5, AAV7, AAV8, and AAV9. In a further embodiment, the AAV is AAV2 or AAV8.

In one embodiment of the first aspect, the AAV is AAV1 and the peptide insertion is immediately after an amino acid selected from amino acids 580 to 595 (VP1 numbering) of AAV1, the AAV is AAV2 and the peptide insertion is immediately after an amino acid selected from amino acids 579 to 594 (VP1 numbering) of AAV2, the AAV is AAV3 and the peptide insertion is immediately after an amino acid selected from amino acids 580 to 595 (VP1 numbering) of AAV3, the AAV is AAV3b and the peptide insertion is immediately after an amino acid selected from amino acids 580 to 595 (VP1 numbering) of AAV3b, the AAV is AAV4 and the peptide insertion is immediately after an amino acid selected from amino acids 578 to 593 (VP1 numbering) of AAV4, the AAV is AAV5 and the peptide insertion is immediately after an amino acid selected from amino acids 569 to 584 (VP1 numbering) of AAV5, the AAV is AAV6 and the peptide insertion is immediately after an amino acid selected from amino acids 580 to 595 (VP1 numbering) of AAV6, the AAV is AAV7 and the peptide insertion is immediately after an amino acid selected from amino acids 581 to 596 (VP1 numbering) of AAV7, the AAV is AAV8 and the peptide insertion is immediately after an amino acid selected from amino acids 582 to 597 (VP1 numbering) of AAV8, the AAV is AAV9 and the peptide insertion is immediately after an amino acid selected from amino acids 580 to 595 (VP1 numbering) of AAV9, the AAV is AAV10 and the peptide insertion is immediately after an amino acid selected from amino acids 582 to 597 (VP1 numbering) AAV10, the AAV is AAV11 and the peptide insertion is immediately after an amino acid selected from amino acids 575 to 593 (VP1 numbering) of AAV11, the AAV is AAV12 and the peptide insertion is immediately after an amino acid selected from amino acids 584 to 600 (VP1 numbering) of AAV12, the AAV is AAV13 and the peptide insertion is immediately after an amino acid selected from amino acids 575 to 593 (VP1 numbering) of AAV13, the AAV is AAVrh10 and the peptide insertion is immediately after an amino acid selected from amino acids 582 to 597 (VP1 numbering) of AAVrh10, or the AAV is AAVrh74 and the peptide insertion is immediately after an amino acid selected from amino acids 582 to 597 (VP1 numbering) of AAVrh74. In some embodiments, the AAV is AAV2 and the peptide insertion is immediately after an amino acid selected from amino acids 579 to 594 (VP1 numbering) of AAV2, the AAV is AAV5 and the peptide insertion is immediately after an amino acid selected from amino acids 569 to 584 (VP1 numbering) of AAV5, the AAV is AAV7 and the peptide insertion is immediately after an amino acid selected from amino acids 581 to 596 (VP1 numbering) of AAV7, the AAV is AAV8 and the peptide insertion is immediately after an amino acid selected from amino acids 582 to 597 (VP1 numbering) of AAV8, or the AAV is AAV9 and the peptide insertion is immediately after an amino acid selected from amino acids 580 to 595 (VP1 numbering) of AAV9. In other embodiments, the AAV is AAV2 and the peptide insertion is immediately after an amino acid selected from amino acids 579 to 594 (VP1 numbering) of AAV2 or the AAV is AAV8 and the peptide insertion is immediately after an amino acid selected from amino acids 582 to 597 (VP1 numbering) of AAV8.

In one embodiment of the first aspect, the AAV is AAV1 and the peptide insertion is immediately after amino acid 588 (VP1 numbering) of AAV1, the AAV is AAV2 and the peptide insertion is immediately after amino acid 587 (VP1 numbering) of AAV2, the AAV is AAV3 and the peptide insertion is immediately after amino acid 588 (VP1 numbering) of AAV3, the AAV is AAV3b and the peptide insertion is immediately after amino acid 588 (VP1 numbering) of AAV3b, the AAV is AAV4 and the peptide insertion is immediately after amino acid 586 (VP1 numbering) of AAV4, the AAV is AAV5 and the peptide insertion is immediately after amino acid 577 (VP1 numbering) of AAV5, the AAV is AAV6 and the peptide insertion is immediately after amino acid 588 (VP1 numbering) of AAV6, the AAV is AAV7 and the peptide insertion is immediately after amino acid 589 (VP1 numbering) of AAV7, the AAV is AAV8 and the peptide insertion is immediately after amino acid 590 (VP1 numbering) of AAV8, the AAV is AAV9 and the peptide insertion is immediately after amino acid 588 (VP1 numbering) of AAV9, the AAV is AAV10 and the peptide insertion is immediately after amino acid 590 (VP1 numbering) of AAV10, the AAV is AAV11 and the peptide insertion is immediately after amino acid 585 (VP1 numbering) of AAV11, the AAV is AAV12 and the peptide insertion is immediately after amino acid 594 (VP1 numbering) of AAV12, the AAV is AAV13 and the peptide insertion is immediately after amino acid 588 (VP1 numbering) of AAV13, the AAV is AAVrh10 and the peptide insertion is immediately after amino acid 590 (VP1 numbering) of AAVrh10, or the AAV is AAVrh74 and the peptide insertion is immediately after amino acid 590 (VP1 numbering) of AAVrh74. In some embodiments, the AAV is AAV2 and the peptide insertion is immediately after amino acid 587 (VP1 numbering) of AAV2, the AAV is AAV5 and the peptide insertion is immediately after amino acid 577 (VP1 numbering) of AAV5, the AAV is AAV7 and the peptide insertion is immediately after amino acid 589 (VP1 numbering) of AAV7, the AAV is AAV8 and the peptide insertion is immediately after amino acid 590 (VP1 numbering) of AAV8, or the AAV is AAV9 and the peptide insertion is immediately after amino acid 588 (VP1 numbering) of AAV9. In other embodiments, the AAV is AAV2 and the peptide insertion is immediately after amino acid 587 (VP1 numbering) of AAV2 or the AAV is AAV8 and the peptide insertion is immediately after amino acid 590 (VP1 numbering) of AAV8.

In one embodiment of the first aspect, the AAV capsid polypeptide excluding the peptide insertion comprises a wild-type AAV capsid amino acid sequence or an amino acid sequence having at least 70% sequence identity thereto. In other embodiments, the sequence identity is at least 75%, at least 80%, at least 85%, at least 90%, at least 95% or at least 98% compared to the wild-type AAV capsid amino acid sequence. The wild-type AAV capsid amino acid sequence can in particular be an AAV1, AAV2, AAV3, AAV3b, AAV4, AAV5, AAV6, AAV7, AAV8, AAV9, AAV10, AAV11, AAV12, AAV13, AAVrh10 or AAVrh74 sequence. In one embodiment, the wild-type AAV capsid amino acid sequence is an AAV2, AAV5, AAV7, AAV8, or AAV9 sequence. In other embodiments, the wild-type AAV capsid amino acid sequence is an AAV2 or AAV8 sequence. In some embodiments, the AAV capsid polypeptide is selected from the group consisting of VP1, VP2 and VP3.

In one embodiment of the first aspect, the AAV capsid polypeptide excluding the peptide insertion consists of a wild-type AAV capsid amino acid sequence or an amino acid sequence having at least 70% sequence identity thereto. In other embodiments, the sequence identity is at least 75%, at least 80%, at least 85%, at least 90%, at least 95% or at least 98% compared to the wild-type AAV capsid amino acid sequence. The wild-type AAV capsid amino acid sequence can in particular be an AAV1, AAV2, AAV3, AAV3b, AAV4, AAV5, AAV6, AAV7, AAV8, AAV9, AAV10, AAV11, AAV12, AAV13, AAVrh10 or AAVrh74 sequence. In one embodiment, the wild-type AAV capsid amino acid sequence is an AAV2, AAV5, AAV7, AAV8, or AAV9 sequence. In other embodiments, the wild-type AAV capsid amino acid sequence is an AAV2 or AAV8 sequence. In some embodiments, the AAV capsid polypeptide is selected from the group consisting of VP1, VP2 and VP3.

In one embodiment of the first aspect, (a) the AAV is AAV1 and the AAV1 capsid polypeptide excluding the peptide insertion comprises or consists of the wild-type capsid amino acid sequence of AAV1 of SEQ ID NO:30 or an amino acid sequence having at least 70% sequence identity thereto, (b) the AAV is AAV2 and the AAV2 capsid polypeptide excluding the peptide insertion comprises or consists of the wild-type capsid amino acid sequence of AAV2 of SEQ ID NO:31 or an amino acid sequence having at least 70% sequence identity thereto, (c) the AAV is AAV3 and the AAV3 capsid polypeptide excluding the peptide insertion comprises or consists of the wild-type capsid amino acid sequence of AAV3 of SEQ ID NO:32 or an amino acid sequence having at least 70% sequence identity thereto, (d) the AAV is AAV3b and the AAV3b capsid polypeptide excluding the peptide insertion comprises or consists of the wild-type capsid amino acid sequence of AAV3b of SEQ ID NO:33 or an amino acid sequence having at least 70% sequence identity thereto, (e) the AAV is AAV4 and the AAV4 capsid polypeptide excluding the peptide insertion comprises or consists of the wild-type capsid amino acid sequence of AAV4 of SEQ ID NO:34 or an amino acid sequence having at least 70% sequence identity thereto, (f) the AAV is AAV5 and the AAV5 capsid polypeptide excluding the peptide insertion comprises or consists of the wild-type capsid amino acid sequence of AAV5 of SEQ ID NO:35 or an amino acid sequence having at least 70% sequence identity thereto, (g) the AAV is AAV6 and the AAV6 capsid polypeptide excluding the peptide insertion comprises or consists of the wild-type capsid amino acid sequence of AAV6 of SEQ ID NO:36 or an amino acid sequence having at least 70% sequence identity thereto, (h) the AAV is AAV7 and the AAV7 capsid polypeptide excluding the peptide insertion comprises or consists of the wild-type capsid amino acid sequence of AAV7 of SEQ ID NO:37 or an amino acid sequence having at least 70% sequence identity thereto, (i) the AAV is AAV8 and the AAV8 capsid polypeptide excluding the peptide insertion comprises or consists of the wild-type capsid amino acid sequence of AAV8 of SEQ ID NO:38 or an amino acid sequence having at least 70% sequence identity thereto, (j) the AAV is AAV9 and the AAV9 capsid polypeptide excluding the peptide insertion comprises or consists of the wild-type capsid amino acid sequence of AAV9 of SEQ ID NO: 39 or an amino acid sequence having at least 70% sequence identity thereto, (k) the AAV is AAV10 and the AAV10 capsid polypeptide excluding the peptide insertion comprises or consists of the wild-type capsid amino acid sequence of AAV10 of SEQ ID NO:40 or an amino acid sequence having at least 70% sequence identity thereto, (I) the AAV is AAV11 and the AAV11 capsid polypeptide excluding the peptide insertion comprises or consists of the wild-type capsid amino acid sequence of AAV11 of SEQ ID NO:41 or an amino acid sequence having at least 70% sequence identity thereto, (m) the AAV is AAV12 and the AAV12 capsid polypeptide excluding the peptide insertion comprises or consists of the wild-type capsid amino acid sequence of AAV12 of SEQ ID NO:42 or an amino acid sequence having at least 70% sequence identity thereto, (n) the AAV is AAV13 and the AAV13 capsid polypeptide excluding the peptide insertion comprises or consists of the wild-type capsid amino acid sequence of AAV13 of SEQ ID NO:43 or an amino acid sequence having at least 70% sequence identity thereto, (o) the AAV is AAVrh10 and the AAVrh10 capsid polypeptide excluding the peptide insertion comprises or consists of the wild-type capsid amino acid sequence of AAVrh10 of SEQ ID NO: 44 or an amino acid sequence having at least 70% sequence identity thereto, or (p) the AAV is AAVrh74 and the AAVrh74 capsid polypeptide excluding the peptide insertion comprises or consists of the wild-type capsid amino acid sequence of AAVrh74 of SEQ ID NO:45 or an amino acid sequence having at least 70% sequence identity thereto. In some embodiments, the sequence identity is at least 75%, at least 80%, at least 85%, at least 90%, at least 95% or at least 98%.

In one embodiment of the first aspect, (a) the AAV is AAV2 and the AAV2 capsid polypeptide excluding the peptide insertion comprises or consists of the wild-type capsid amino acid sequence of AAV2 of SEQ ID NO:31 or an amino acid sequence having at least 70% sequence identity thereto, (b) the AAV is AAV5 and the AAV5 capsid polypeptide excluding the peptide insertion comprises or consists of the wild-type capsid amino acid sequence of AAV5 of SEQ ID NO:35 or an amino acid sequence having at least 70% sequence identity thereto, (c) the AAV is AAV7 and the AAV7 capsid polypeptide excluding the peptide insertion comprises or consists of the wild-type capsid amino acid sequence of AAV7 of SEQ ID NO:37 or an amino acid sequence having at least 70% sequence identity thereto, (d) the AAV is AAV8 and the AAV8 capsid polypeptide excluding the peptide insertion comprises or consists of the wild-type capsid amino acid sequence of AAV8 of SEQ ID NO:38 or an amino acid sequence having at least 70% sequence identity thereto, or (e) the AAV is AAV9 and the AAV9 capsid polypeptide excluding the peptide insertion comprises or consists of the wild-type capsid amino acid sequence of AAV9 of SEQ ID NO:39 or an amino acid sequence having at least 70% sequence identity thereto. In some embodiments, the sequence identity is at least 75%, at least 80%, at least 85%, at least 90%, at least 95% or at least 98%.

In one embodiment of the first aspect, (a) the AAV is AAV2 and the AAV2 capsid polypeptide excluding the peptide insertion comprises or consists of the wild-type capsid amino acid sequence of AAV2 of SEQ ID NO:31 or an amino acid sequence having at least 70% sequence identity thereto, or (b) the AAV is AAV8 and the AAV8 capsid polypeptide excluding the peptide insertion comprises or consists of the wild-type capsid amino acid sequence of AAV8 of SEQ ID NO:38 or an amino acid sequence having at least 70% sequence identity thereto. In some embodiments, the sequence identity is at least 75%, at least 80%, at least 85%, at least 90%, at least 95% or at least 98%.

In one embodiment of the first aspect, the amino acid (immediately) preceding the site into which said peptide is inserted has been changed, if applicable, to R or S. In one embodiment of the first aspect, the two amino acids (immediately) preceding the site into which said peptide is inserted have been changed, if applicable, to QR or QS. In one embodiment of the first aspect, the three amino acids (immediately) preceding the site into which said peptide is inserted have been changed, if applicable, to GOR or GQS. These changes are to be seen relative to the wild-type AAV capsid polypeptide amino acid sequence.

In one embodiment of the first aspect, the amino acid (immediately) following the site into which said peptide is inserted has been changed, if applicable, to Q. In one embodiment of the first aspect, the two amino acids (immediately) following the site into which said peptide is inserted have been changed, if applicable, to QA. In one embodiment of the first aspect, the three amino acids (immediately) following the site into which said peptide is inserted have been changed, if applicable, to QAA. These changes are to be seen relative to the wild-type AAV capsid polypeptide amino acid sequence.

In one embodiment of the first aspect, the amino acid (immediately) preceding the site into which said peptide is inserted has been changed, if applicable, to R or S, and the amino acid (immediately) following the site into which said peptide is inserted has been changed, if applicable, to Q. In one embodiment of the first aspect, the two amino acids (immediately) preceding the site into which said peptide is inserted have been changed, if applicable, to QR or QS, and the two amino acids (immediately) following the site into which said peptide is inserted have been changed, if applicable, to QA. In one embodiment of the first aspect, the three amino acids (immediately) preceding the site into which said peptide is inserted have been changed, if applicable, to GOR or GQS, and the three amino acids (immediately) following the site into which said peptide is inserted have been changed, if applicable, to QAA. These changes are to be seen relative to the wild-type AAV capsid polypeptide amino acid sequence.

In one embodiment of the first aspect, (a) the AAV is AAV1 and the AAV1 capsid polypeptide excluding the peptide insertion comprises or consists of the amino acid sequence of SEQ ID NO:46 or 47 or an amino acid sequence having at least 70% sequence identity thereto, (b) the AAV is AAV2 and the AAV2 capsid polypeptide excluding the peptide insertion comprises or consists of the amino acid sequence of SEQ ID NO:48 or 49 or an amino acid sequence having at least 70% sequence identity thereto, (c) the AAV is AAV3 and the AAV3 capsid polypeptide excluding the peptide insertion comprises or consists of the amino acid sequence of SEQ ID NO:50 or 51 or an amino acid sequence having at least 70% sequence identity thereto, (d) the AAV is AAV3b and the AAV3b capsid polypeptide excluding the peptide insertion comprises or consists of the amino acid sequence of SEQ ID NO: 52 or 53 or an amino acid sequence having at least 70% sequence identity thereto, (e) the AAV is AAV4 and the AAV4 capsid polypeptide excluding the peptide insertion comprises or consists of the amino acid sequence of SEQ ID NO:54 or 55 or an amino acid sequence having at least 70% sequence identity thereto, (f) the AAV is AAV5 and the AAV5 capsid polypeptide excluding the peptide insertion comprises or consists of the amino acid sequence of SEQ ID NO:56 or 57 or an amino acid sequence having at least 70% sequence identity thereto, (g) the AAV is AAV6 and the AAV6 capsid polypeptide excluding the peptide insertion comprises or consists of the amino acid sequence of SEQ ID NO: 58 or 59 or an amino acid sequence having at least 70% sequence identity thereto, (h) the AAV is AAV7 and the AAV7 capsid polypeptide excluding the peptide insertion comprises or consists of the amino acid sequence of SEQ ID NO:60 or 61 or an amino acid sequence having at least 70% sequence identity thereto, (i) the AAV is AAV8 and the AAV8 capsid polypeptide excluding the peptide insertion comprises or consists of the amino acid sequence of SEQ ID NO:62 or 63 or an amino acid sequence having at least 70% sequence identity thereto, (j) the AAV is AAV9 and the AAV9 capsid polypeptide excluding the peptide insertion comprises or consists of the amino acid sequence of SEQ ID NO: 64 or 65 or an amino acid sequence having at least 70% sequence identity thereto, (k) the AAV is AAV10 and the AAV10 capsid polypeptide excluding the peptide insertion comprises or consists of the amino acid sequence of SEQ ID NO:66 or 67 or an amino acid sequence having at least 70% sequence identity thereto, (I) the AAV is AAV11 and the AAV11 capsid polypeptide excluding the peptide insertion comprises or consists of the amino acid sequence of SEQ ID NO:68 or 69 or an amino acid sequence having at least 70% sequence identity thereto, (m) the AAV is AAV12 and the AAV12 capsid polypeptide excluding the peptide insertion comprises or consists of the amino acid sequence of SEQ ID NO:70 or 71 or an amino acid sequence having at least 70% sequence identity thereto, (n) the AAV is AAV13 and the AAV13 capsid polypeptide excluding the peptide insertion comprises or consists of the amino acid sequence of SEQ ID NO:72 or 73 or an amino acid sequence having at least 70% sequence identity thereto, (o) the AAV is AAVrh10 and the AAVrh10 capsid polypeptide excluding the peptide insertion comprises or consists of the amino acid sequence of SEQ ID NO: 74 or 75 or an amino acid sequence having at least 70% sequence identity thereto, or (p) the AAV is AAVrh74 and the AAVrh74 capsid polypeptide excluding the peptide insertion comprises or consists of the amino acid sequence of SEQ ID NO:76 or 77 or an amino acid sequence having at least 70% sequence identity thereto. In some embodiments, the sequence identity is at least 75%, at least 80%, at least 85%, at least 90%, at least 95% or at least 98%.

In one embodiment of the first aspect, (a) the AAV is AAV2 and the AAV2 capsid polypeptide excluding the peptide insertion comprises or consists of the amino acid sequence of SEQ ID NO:48 or 49 or an amino acid sequence having at least 70% sequence identity thereto, (b) the AAV is AAV5 and the AAV5 capsid polypeptide excluding the peptide insertion comprises or consists of the amino acid sequence of SEQ ID NO:56 or 57 or an amino acid sequence having at least 70% sequence identity thereto, (c) the AAV is AAV7 and the AAV7 capsid polypeptide excluding the peptide insertion comprises or consists of the amino acid sequence of SEQ ID NO:60 or 61 or an amino acid sequence having at least 70% sequence identity thereto, (d) the AAV is AAV8 and the AAV8 capsid polypeptide excluding the peptide insertion comprises or consists of the amino acid sequence of SEQ ID NO:62 or 63 or an amino acid sequence having at least 70% sequence identity thereto, or (e) the AAV is AAV9 and the AAV9 capsid polypeptide excluding the peptide insertion comprises or consists of the amino acid sequence of SEQ ID NO:64 or 65 or an amino acid sequence having at least 70% sequence identity thereto. In some embodiments, the sequence identity is at least 75%, at least 80%, at least 85%, at least 90%, at least 95% or at least 98%.

In one embodiment of the first aspect, (a) the AAV is AAV2 and the AAV2 capsid polypeptide excluding the peptide insertion comprises or consists of the amino acid sequence of SEQ ID NO:48 or 49 or an amino acid sequence having at least 70% sequence identity thereto, or (b) the AAV is AAV8 and the AAV8 capsid polypeptide excluding the peptide insertion comprises or consists of the amino acid sequence of SEQ ID NO:62 or 63 or an amino acid sequence having at least 70% sequence identity thereto. It can be preferred that the sequence identity is at least 75%, at least 80%, at least 85%, at least 90%, at least 95% or at least 98%.

### Embodiments Relating to Combinations

In one embodiment of the first aspect, the peptide insertion (i) comprises an amino acid sequence selected from the group consisting of SEQ ID NOs: 1-29 or (ii) consists of an amino acid sequence of a G at the N-terminus, followed by an amino acid sequence selected from the group consisting of SEQ ID NOs: 1-29, followed by an A at the C-terminus, and (a) the AAV is AAV1 and the AAV1 capsid polypeptide excluding the peptide insertion comprises or consists of the wild-type capsid amino acid sequence of AAV1 of SEQ ID NO:30 or an amino acid sequence having at least 80% sequence identity thereto, and the peptide insertion is immediately after an amino acid selected from amino acids 580 to 595 (VP1 numbering) of AAV1, (b) the AAV is AAV2 and the AAV2 capsid polypeptide excluding the peptide insertion comprises or consists of the wild-type capsid amino acid sequence of AAV2 of SEQ ID NO:31 or an amino acid sequence having at least 80% sequence identity thereto, and the peptide insertion is immediately after an amino acid selected from amino acids 579 to 594 (VP1 numbering) of AAV2, (c) the AAV is AAV3 and the AAV3 capsid polypeptide excluding the peptide insertion comprises or consists of the wild-type capsid amino acid sequence of AAV3 of SEQ ID NO:32 or an amino acid sequence having at least 80% sequence identity thereto, and the peptide insertion is immediately after an amino acid selected from amino acids 580 to 595 (VP1 numbering) of AAV3, (d) the AAV is AAV3b and the AAV3b capsid polypeptide excluding the peptide insertion comprises or consists of the wild-type capsid amino acid sequence of AAV3b of SEQ ID NO:33 or an amino acid sequence having at least 80% sequence identity thereto, and the peptide insertion is immediately after an amino acid selected from amino acids 580 to 595 (VP1 numbering) of AAV3b, (e) the AAV is AAV4 and the AAV4 capsid polypeptide excluding the peptide insertion comprises or consists of the wild-type capsid amino acid sequence of AAV4 of SEQ ID NO:34 or an amino acid sequence having at least 80% sequence identity thereto, and the peptide insertion is immediately after an amino acid selected from amino acids 578 to 593 (VP1 numbering) of AAV4, (f) the AAV is AAV5 and the AAV5 capsid polypeptide excluding the peptide insertion comprises or consists of the wild-type capsid amino acid sequence of AAV5 of SEQ ID NO:35 or an amino acid sequence having at least 80% sequence identity thereto, and the peptide insertion is immediately after an amino acid selected from amino acids 569 to 584 (VP1 numbering) of AAV5, (g) the AAV is AAV6 and the AAV6 capsid polypeptide excluding the peptide insertion comprises or consists of the wild-type capsid amino acid sequence of AAV6 of SEQ ID NO:36 or an amino acid sequence having at least 80% sequence identity thereto, and the peptide insertion is immediately after an amino acid selected from amino acids 580 to 595 (VP1 numbering) of AAV6, (h) the AAV is AAV7 and the AAV7 capsid polypeptide excluding the peptide insertion comprises or consists of the wild-type capsid amino acid sequence of AAV7 of SEQ ID NO:37 or an amino acid sequence having at least 80% sequence identity thereto, and the peptide insertion is immediately after an amino acid selected from amino acids 581 to 596 (VP1 numbering) of AAV7, (i) the AAV is AAV8 and the AAV8 capsid polypeptide excluding the peptide insertion comprises or consists of the wild-type capsid amino acid sequence of AAV8 of SEQ ID NO:38 or an amino acid sequence having at least 80% sequence identity thereto, and the peptide insertion is immediately after an amino acid selected from amino acids 582 to 597 (VP1 numbering) of AAV8, (j) the AAV is AAV9 and the AAV9 capsid polypeptide excluding the peptide insertion comprises or consists of the wild-type capsid amino acid sequence of AAV9 of SEQ ID NO:39 or an amino acid sequence having at least 80% sequence identity thereto, and the peptide insertion is immediately after an amino acid selected from amino acids 580 to 595 (VP1 numbering) of AAV9, (k) the AAV is AAV10 and the AAV10 capsid polypeptide excluding the peptide insertion comprises or consists of the wild-type capsid amino acid sequence of AAV10 of SEQ ID NO:40 or an amino acid sequence having at least 80% sequence identity thereto, and the peptide insertion is immediately after an amino acid selected from amino acids 582 to 597 (VP1 numbering) AAV10, (I) the AAV is AAV11 and the AAV11 capsid polypeptide excluding the peptide insertion comprises or consists of the wild-type capsid amino acid sequence of AAV11 of SEQ ID NO:41 or an amino acid sequence having at least 80% sequence identity thereto, and the peptide insertion is immediately after an amino acid selected from amino acids 575 to 593 (VP1 numbering) of AAV11, (m) the AAV is AAV12 and the AAV12 capsid polypeptide excluding the peptide insertion comprises or consists of the wild-type capsid amino acid sequence of AAV12 of SEQ ID NO: 42 or an amino acid sequence having at least 80% sequence identity thereto, and the peptide insertion is immediately after an amino acid selected from amino acids 584 to 600 (VP1 numbering) of AAV12, (n) the AAV is AAV13 and the AAV13 capsid polypeptide excluding the peptide insertion comprises or consists of the wild-type capsid amino acid sequence of AAV13 of SEQ ID NO:43 or an amino acid sequence having at least 80% sequence identity thereto, and the peptide insertion is immediately after an amino acid selected from amino acids 575 to 593 (VP1 numbering) of AAV13, (o) the AAV is AAVrh10 and the AAVrh10 capsid polypeptide excluding the peptide insertion comprises or consists of the wild-type capsid amino acid sequence of AAVrh10 of SEQ ID NO:44 or an amino acid sequence having at least 80% sequence identity thereto, and the peptide insertion is immediately after an amino acid selected from amino acids 582 to 597 (VP1 numbering) of AAVrh10, or the AAV is AAVrh74 and the AAVrh74 capsid polypeptide excluding the peptide insertion comprises or consists of the wild-type capsid amino acid sequence of AAVrh74 of SEQ ID NO:45 or an amino acid sequence having at least 80% sequence identity thereto, and the peptide insertion is immediately after an amino acid selected from amino acids 582 to 597 (VP1 numbering) of AAVrh74. As regards (i) of the present embodiment, in particular embodiments, the peptide insertion comprises an amino acid sequence selected from the group consisting of SEQ ID NOs: 1-10. In other embodiments, the peptide insertion comprises the amino acid sequence of SEQ ID NO:1 or SEQ ID NO:2 or SEQ ID NO: 3 or SEQ ID NO:4 or SEQ ID NO:5 or SEQ ID NO:6 or SEQ ID NO:7. In other embodiments the peptide insertion comprises the amino acid sequence of SEQ ID NO:1 or SEQ ID NO:2. In other embodiments the peptide insertion comprises the amino acid sequence of SEQ ID NO:2 or SEQ ID NO: 10. As regards (ii) of the present embodiment, in particular embodiments, the peptide insertion consists of an amino acid sequence of a G at the N-terminus, followed by an amino acid sequence selected from the group consisting of SEQ ID NOs: 1-10, followed by an A at the C-terminus; more preferably the peptide insertion consists of an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, followed by an A at the C-terminus; most preferably the peptide insertion consists of an amino acid sequence of a G at the N-terminus, followed by the amino acid sequence of SEQ ID NO:1 or SEQ ID NO:2, followed by an A at the C-terminus. In other embodiments, the peptide insertion consists of an amino acid sequence of a G at the N-terminus, followed by the amino acid sequence of SEQ ID NO:2 or SEQ ID NO:10, followed by an A at the C-terminus. In some embodiments, the sequence identity of the amino acid sequence having sequence identity to the AAV capsid polypeptide excluding the peptide insertion is at least 85%, at least 90%, at least 95% or at least 98%. Further, in other embodiments, the AAV is AAV2, AAV5, AAV7, AAV8 or AAV9 with the sequence and insertion site as defined in this paragraph. In other embodiments, the AAV is AAV2 or AAV8 with the sequence and insertion site as defined in this paragraph.

In one embodiment of the first aspect, the peptide insertion (i) comprises an amino acid sequence selected from the group consisting of SEQ ID NOs: 1-29 or (ii) consists of an amino acid sequence of a G at the N-terminus, followed by an amino acid sequence selected from the group consisting of SEQ ID NOs: 1-29, followed by an A at the C-terminus, and (a) the AAV is AAV1 and the AAV1 capsid polypeptide excluding the peptide insertion comprises or consists of the wild-type capsid amino acid sequence of AAV1 of SEQ ID NO:30 or an amino acid sequence having at least 80% sequence identity thereto, and the peptide insertion is immediately after amino acid 588 (VP1 numbering) of AAV1, (b) the AAV is AAV2 and the AAV2 capsid polypeptide excluding the peptide insertion comprises or consists of the wild-type capsid amino acid sequence of AAV2 of SEQ ID NO:31 or an amino acid sequence having at least 80% sequence identity thereto, and the peptide insertion is immediately after amino acid 587 (VP1 numbering) of AAV2, (c) the AAV is AAV3 and the AAV3 capsid polypeptide excluding the peptide insertion comprises or consists of the wild-type capsid amino acid sequence of AAV3 of SEQ ID NO:32 or an amino acid sequence having at least 80% sequence identity thereto, and the peptide insertion is immediately after amino acid 588 (VP1 numbering) of AAV3, (d) the AAV is AAV3b and the AAV3 capsid polypeptide excluding the peptide insertion comprises or consists of the wild-type capsid amino acid sequence of AAV3b of SEQ ID NO:33 or an amino acid sequence having at least 80% sequence identity thereto, and the peptide insertion is immediately after amino acid 588 (VP1 numbering) of AAV3b, (e) the AAV is AAV4 and the AAV4 capsid polypeptide excluding the peptide insertion comprises or consists of the wild-type capsid amino acid sequence of AAV4 of SEQ ID NO:34 or an amino acid sequence having at least 80% sequence identity thereto, and the peptide insertion is immediately after amino acid 586 (VP1 numbering) of AAV4, (f) the AAV is AAV5 and the AAV5 capsid polypeptide excluding the peptide insertion comprises or consists of the wild-type capsid amino acid sequence of AAV5 of SEQ ID NO:35 or an amino acid sequence having at least 80% sequence identity thereto, and the peptide insertion is immediately after amino acid 577 (VP1 numbering) of AAV5, (g) the AAV is AAV6 and the AAV6 capsid polypeptide excluding the peptide insertion comprises or consists of the wild-type capsid amino acid sequence of AAV6 of SEQ ID NO: 36 or an amino acid sequence having at least 80% sequence identity thereto, and the peptide insertion is immediately after amino acid 588 (VP1 numbering) of AAV6, (h) the AAV is AAV7 and the AAV7 capsid polypeptide excluding the peptide insertion comprises or consists of the wild-type capsid amino acid sequence of AAV7 of SEQ ID NO:37 or an amino acid sequence having at least 80% sequence identity thereto, and the peptide insertion is immediately after amino acid 589 (VP1 numbering) of AAV7, (i) the AAV is AAV8 and the AAV8 capsid polypeptide excluding the peptide insertion comprises or consists of the wild-type capsid amino acid sequence of AAV8 of SEQ ID NO:38 or an amino acid sequence having at least 80% sequence identity thereto, and the peptide insertion is immediately after amino acid 590 (VP1 numbering) of AAV8, (j) the AAV is AAV9 and the AAV9 capsid polypeptide excluding the peptide insertion comprises or consists of the wild-type capsid amino acid sequence of AAV9 of SEQ ID NO:39 or an amino acid sequence having at least 80% sequence identity thereto, and the peptide insertion is immediately after amino acid 588 (VP1 numbering) of AAV9, (k) the AAV is AAV10 and the AAV10 capsid polypeptide excluding the peptide insertion comprises or consists of the wild-type capsid amino acid sequence of AAV10 of SEQ ID NO:40 or an amino acid sequence having at least 80% sequence identity thereto, and the peptide insertion is immediately after amino acid 590 (VP1 numbering) of AAV10, (I) the AAV is AAV11 and the AAV11 capsid polypeptide excluding the peptide insertion comprises or consists of the wild-type capsid amino acid sequence of AAV11 of SEQ ID NO:41 or an amino acid sequence having at least 80% sequence identity thereto, and the peptide insertion is immediately after amino acid 585 (VP1 numbering) of AAV11, (m) the AAV is AAV12 and the AAV12 capsid polypeptide excluding the peptide insertion comprises or consists of the wild-type capsid amino acid sequence of AAV12 of SEQ ID NO:42 or an amino acid sequence having at least 80% sequence identity thereto, and the peptide insertion is immediately after amino acid 594 (VP1 numbering) of AAV12, (n) the AAV is AAV13 and the AAV13 capsid polypeptide excluding the peptide insertion comprises or consists of the wild-type capsid amino acid sequence of AAV13 of SEQ ID NO:43 or an amino acid sequence having at least 80% sequence identity thereto, and the peptide insertion is immediately after amino acid 588 (VP1 numbering) of AAV13, (o) the AAV is AAVrh10 and the AAVrh10 capsid polypeptide excluding the peptide insertion comprises or consists of the wild-type capsid amino acid sequence of AAVrh10 of SEQ ID NO:44 or an amino acid sequence having at least 80% sequence identity thereto, and the peptide insertion is immediately after amino acid 590 (VP1 numbering) of AAVrh10, or (p) the AAV is AAVrh74 and the AAVrh74 capsid polypeptide excluding the peptide insertion comprises or consists of the wild-type capsid amino acid sequence of AAVrh74 of SEQ ID NO:45 or an amino acid sequence having at least 80% sequence identity thereto, and the peptide insertion is immediately after amino acid 590 (VP1 numbering) of AAVrh74. As regards (i) of the present embodiment, in some embodiments, the peptide insertion comprises an amino acid sequence selected from the group consisting of SEQ ID NOs: 1-10.

In other embodiments, the peptide insertion comprises the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, most preferably the peptide insertion comprises the amino acid sequence of SEQ ID NO:1 or SEQ ID NO: 2. In other embodiments, the peptide insertion comprises the amino acid sequence of SEQ ID NO: 2 or SEQ ID NO:10. As regards (ii) of the present embodiment, In some embodiments, the peptide insertion consists of an amino acid sequence of a G at the N-terminus, followed by an amino acid sequence selected from the group consisting of SEQ ID NOs: 1-10, followed by an A at the C-terminus. In other embodiments, the peptide insertion consists of an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, followed by an A at the C-terminus; most preferably the peptide insertion consists of an amino acid sequence of a G at the N-terminus, followed by the amino acid sequence of SEQ ID NO:1 or SEQ ID NO:2, followed by an A at the C-terminus. In other embodiments, the peptide insertion consists of an amino acid sequence of a G at the N-terminus, followed by the amino acid sequence of SEQ ID NO:2 or SEQ ID NO:10, followed by an A at the C-terminus. In some embodiments, the sequence identity of the amino acid sequence having sequence identity to the AAV capsid polypeptide excluding the peptide insertion is at least 85%, at least 90%, at least 95% or at least 98%. Further, in other embodiments, the AAV is AAV2, AAV5, AAV7, AAV8 or AAV9 with the sequence and insertion site as defined in this paragraph. In other embodiments, the AAV is AAV2 or AAV8 with the sequence and insertion site as defined in this paragraph.

In one embodiment of the first aspect, the peptide insertion (i) comprises an amino acid sequence selected from the group consisting of SEQ ID NOs: 1-29 or (ii) consists of an amino acid sequence of a G at the N-terminus, followed by an amino acid sequence selected from the group consisting of SEQ ID NOs: 1-29, followed by an A at the C-terminus, and (a) the AAV is AAV1 and the AAV1 capsid polypeptide excluding the peptide insertion comprises or consists of the amino acid sequence of SEQ ID NO: 46 or 47 or an amino acid sequence having at least 80% sequence identity thereto, and the peptide insertion is immediately after amino acid 588 (VP1 numbering) of AAV1, (b) the AAV is AAV2 and the AAV2 capsid polypeptide excluding the peptide insertion comprises or consists of the amino acid sequence of SEQ ID NO:48 or 49 or an amino acid sequence having at least 80% sequence identity thereto, and the peptide insertion is immediately after amino acid 587 (VP1 numbering) of AAV2, (c) the AAV is AAV3 and the AAV3 capsid polypeptide excluding the peptide insertion comprises or consists of the amino acid sequence SEQ ID NO:50 or 51 or an amino acid sequence having at least 80% sequence identity thereto, and the peptide insertion is immediately after amino acid 588 (VP1 numbering) of AAV3, (d) the AAV is AAV3b and the AAV3b capsid polypeptide excluding the peptide insertion comprises or consists of the amino acid sequence SEQ ID NO:52 or 53 or an amino acid sequence having at least 80% sequence identity thereto, and the peptide insertion is immediately after amino acid 588 (VP1 numbering) of AAV3b, (e) the AAV is AAV4 and the AAV4 capsid polypeptide excluding the peptide insertion comprises or consists of the amino acid sequence of SEQ ID NO:54 or 55 or an amino acid sequence having at least 80% sequence identity thereto, and the peptide insertion is immediately after amino acid 586 (VP1 numbering) of AAV4, (f) the AAV is AAV5 and the AAV5 capsid polypeptide excluding the peptide insertion comprises or consists of the amino acid sequence of SEQ ID NO:56 or 57 or an amino acid sequence having at least 80% sequence identity thereto, and the peptide insertion is immediately after amino acid 577 (VP1 numbering) of AAV5, (g) the AAV is AAV6 and the AAV6 capsid polypeptide excluding the peptide insertion comprises or consists of the amino acid sequence of SEQ ID NO:58 or 59 or an amino acid sequence having at least 80% sequence identity thereto, and the peptide insertion is immediately after amino acid 588 (VP1 numbering) of AAV6, (h) the AAV is AAV7 and the AAV7 capsid polypeptide excluding the peptide insertion comprises or consists of the amino acid sequence of SEQ ID NO:60 or 61 or an amino acid sequence having at least 80% sequence identity thereto, and the peptide insertion is immediately after amino acid 589 (VP1 numbering) of AAV7, (i) the AAV is AAV8 and the AAV8 capsid polypeptide excluding the peptide insertion comprises or consists of the amino acid sequence of SEQ ID NO:62 or 63 or an amino acid sequence having at least 80% sequence identity thereto, and the peptide insertion is immediately after amino acid 590 (VP1 numbering) of AAV8, (j) the AAV is AAV9 and the AAV9 capsid polypeptide excluding the peptide insertion comprises or consists of the amino acid sequence of SEQ ID NO:64 or 65 or an amino acid sequence having at least 80% sequence identity thereto, and the peptide insertion is immediately after amino acid 588 (VP1 numbering) of AAV9, (k) the AAV is AAV10 and the AAV10 capsid polypeptide excluding the peptide insertion comprises or consists of the amino acid sequence of SEQ ID NO:66 or 67 or an amino acid sequence having at least 80% sequence identity thereto, and the peptide insertion is immediately after amino acid 590 (VP1 numbering) of AAV10, (I) the AAV is AAV11 and the AAV11 capsid polypeptide excluding the peptide insertion comprises or consists of the amino acid sequence of SEQ ID NO:68 or 69 or an amino acid sequence having at least 80% sequence identity thereto, and the peptide insertion is immediately after amino acid 585 (VP1 numbering) of AAV11, (m) the AAV is AAV12 and the AAV12 capsid polypeptide excluding the peptide insertion comprises or consists of the amino acid sequence of SEQ ID NO:70 or 71 or an amino acid sequence having at least 80% sequence identity thereto, and the peptide insertion is immediately after amino acid 594 (VP1 numbering) of AAV12, (n) the AAV is AAV13 and the AAV13 capsid polypeptide excluding the peptide insertion comprises or consists of the amino acid sequence of SEQ ID NO:72 or 73 or an amino acid sequence having at least 80% sequence identity thereto, and the peptide insertion is immediately after amino acid 588 (VP1 numbering) of AAV13, (0) the AAV is AAVrh10 and the AAVrh10 capsid polypeptide excluding the peptide insertion comprises or consists of the amino acid sequence of SEQ ID NO: 74 or 75 or an amino acid sequence having at least 80% sequence identity thereto, and the peptide insertion is immediately after amino acid 590 (VP1 numbering) of AAVrh10, or (p) the AAV is AAVrh74 and the AAVrh74 capsid polypeptide excluding the peptide insertion comprises or consists of the amino acid sequence of SEQ ID NO:76 or 77 or an amino acid sequence having at least 80% sequence identity thereto, and the peptide insertion is immediately after amino acid 590 (VP1 numbering) of AAVrh74. As regards (i) of the present embodiment, in some embodiments, the peptide insertion comprises an amino acid sequence selected from the group consisting of SEQ ID NOs: 1-10. In other embodiments, the peptide insertion comprises the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7. In other embodiments, the peptide insertion comprises the amino acid sequence of SEQ ID NO:1 or SEQ ID NO: 2. In other embodiments, the peptide insertion comprises the amino acid sequence of SEQ ID NO: 2 or SEQ ID NO:10. As regards (ii) of the present embodiment, in some embodiments, the peptide insertion consists of an amino acid sequence of a G at the N-terminus, followed by an amino acid sequence selected from the group consisting of SEQ ID NOs: 1-10, followed by an A at the C-terminus. In other embodiments, the peptide insertion consists of an amino acid sequence of a G at the N-terminus, followed by the amino acid sequence of SEQ ID NO:1 or SEQ ID NO:2 or SEQ ID NO:3 or SEQ ID NO:4 or SEQ ID NO:5 or SEQ ID NO:6 or SEQ ID NO:7, followed by an A at the C-terminus.

In further embodiments, the peptide insertion consists of an amino acid sequence of a G at the N-terminus, followed by the amino acid sequence of SEQ ID NO:1 or SEQ ID NO:2, followed by an A at the C-terminus. In other embodiments, the peptide insertion consists of an amino acid sequence of a G at the N-terminus, followed by the amino acid sequence of SEQ ID NO:2 or SEQ ID NO:10, followed by an A at the C-terminus. In some embodiments, the sequence identity of the amino acid sequence having sequence identity to the AAV capsid polypeptide excluding the peptide insertion is at least 85%, at least 90%, at least 95% or at least 98%. Further, in other embodiments, the AAV is AAV2, AAV5, AAV7, AAV8 or AAV9 with the sequence and insertion site as defined in this paragraph. In other embodiments, the AAV is AAV2 or AAV8 with the sequence and insertion site as defined in this paragraph.

In one embodiment, the peptide insertion (i) comprises an amino acid sequence selected from the group consisting of SEQ ID NOs: 1-10 or (ii) consists of an amino acid sequence of a G at the N-terminus, followed by an amino acid sequence selected from the group consisting of SEQ ID NOs: 1-10, followed by an A at the C-terminus, and (a) the AAV is AAV2 and the AAV2 capsid polypeptide excluding the peptide insertion comprises or consists of the amino acid sequence of SEQ ID NO:48 or 49 or an amino acid sequence having at least 90% sequence identity thereto, and the peptide insertion is immediately after amino acid 587 (VP1 numbering) of AAV2 or (b) the AAV is AAV8 and the AAV8 capsid polypeptide excluding the peptide insertion comprises or consists of the amino acid sequence of SEQ ID NO:62 or 63 or an amino acid sequence having at least 90% sequence identity thereto, and the peptide insertion is immediately after amino acid 590 (VP1 numbering) of AAV8.

In one embodiment, the peptide insertion (i) comprises the amino acid sequence with SEQ ID NO:1 or SEQ ID NO:2, or (ii) consists of an amino acid sequence of a G at the N-terminus, followed by the amino acid sequence with SEQ ID NO:1 or SEQ ID NO:2, followed by an A at the C-terminus, and (a) the AAV is AAV2 and the AAV2 capsid polypeptide excluding the peptide insertion comprises or consists of the amino acid sequence of SEQ ID NO:46 or an amino acid sequence having at least 90% sequence identity thereto, and the peptide insertion is immediately after amino acid 587 (VP1 numbering) of AAV2 or (b) the AAV is AAV8 and the AAV8 capsid polypeptide excluding the peptide insertion comprises or consists of the amino acid sequence of SEQ ID NO:62 or an amino acid sequence having at least 90% sequence identity thereto, and the peptide insertion is immediately after amino acid 590 (VP1 numbering) of AAV8.

In one embodiment, the peptide insertion (i) comprises the amino acid sequence with SEQ ID NO:2 or SEQ ID NO:10, or (ii) consists of an amino acid sequence of a G at the N-terminus, followed by the amino acid sequence with SEQ ID NO:2 or SEQ ID NO:10, followed by an A at the C-terminus, and (a) the AAV is AAV2 and the AAV2 capsid polypeptide excluding the peptide insertion comprises or consists of the amino acid sequence of SEQ ID NO:46 or an amino acid sequence having at least 90% sequence identity thereto, and the peptide insertion is immediately after amino acid 587 (VP1 numbering) of AAV2 or (b) the AAV is AAV8 and the AAV8 capsid polypeptide excluding the peptide insertion comprises or consists of the amino acid sequence of SEQ ID NO:62 or an amino acid sequence having at least 90% sequence identity thereto, and the peptide insertion is immediately after amino acid 590 (VP1 numbering) of AAV8.

In a second aspect, the present disclosure is directed to an isolated nucleic acid encoding the variant polypeptide according to the first aspect (including all embodiments as outlined above).

In a third aspect, the present disclosure is directed to a recombinant polynucleotide comprising the nucleic acid according to the second aspect (including all embodiments as outlined above).

In one embodiment of the third aspect, the recombinant polynucleotide is useful for delivering the isolated nucleic acid of the second aspect to cells in vitro or in vivo and imparting the ability to express the encoded variant polypeptide according to the first aspect to the cells. Then, the cell to which the nucleic acid of the present disclosure is delivered, is useful for producing an AAV vector.

In one embodiment of the third aspect, the recombinant polynucleotide is DNA.

In a fourth aspect, the present disclosure is directed to an isolated cell comprising the variant polypeptide according to the first aspect (including all embodiments as outlined above), the isolated nucleic acid according to the second aspect (including all embodiments as outlined above) or the recombinant polynucleotide according to the third aspect (including all embodiments as outlined above).

In one embodiment of the fourth aspect, the isolated cell is an isolated host cell.

The isolated host cell of the fourth aspect can in particular be used for production of the AAV vector of the present disclosure. When the isolated host cell of the present disclosure is used for producing an AAV vector, the host cell may be referred to as a “packaging cell” or “producer cell”.

In one embodiment of the fourth aspect, the host cell comprises the recombinant polynucleotide of the third aspect integrated into the genome or retains the recombinant polynucleotide of the third aspect in the cell, so as to transiently express the variant AAV capsid polypeptide.

In a fifth aspect, the present disclosure is directed to an adeno-associated virus (AAV) vector comprising the variant polypeptide according to the first aspect (including all embodiments as outlined above).

In one embodiment of the fifth aspect, the AAV vector comprises (i) a variant AAV VP1 capsid polypeptide according to the first aspect (including all embodiments as outline above), (ii) a variant AAV VP2 capsid polypeptide according to the first aspect (including all embodiments as outlined above), and (iii) a variant AAV VP3 capsid polypeptide according to the first aspect (including all embodiments thereof), preferably in a stochiometric ratio of VP1:VP2:VP3 of about 1:1:10. In another embodiment of the fifth aspect, the AAV vector comprises (i) a variant AAV VP1 capsid polypeptide according to the first aspect (including all embodiments as outline above), or (ii) a variant AAV VP2 capsid polypeptide according to the first aspect (including all embodiments as outlined above), or (iii) a variant AAV VP3 capsid polypeptide according to the first aspect (including all embodiments thereof) or combinations thereof.

In one embodiment of the fifth aspect, the AAV vector further comprises a heterologous nucleic acid. In some embodiments, the heterologous nucleic acid is a ssDNA, a partially complementary dsDNA or a ssRNA. In other embodiments, the heterologous nucleic acid comprises two or three inverted terminal repeat sequences (ITRs), for example one ITR at each of its 5′ and 3′ end. The skilled person is aware of ITR sequences, in particular AAV ITR sequences that are typically used. AAV ITR sequences that can be used can be derived from any AAV serotype and can have a wild-type nucleotide sequence or may be altered (and thus e.g., include an insertion, deletion or substitution of at least one nucleotide). Furthermore, 5′ and 3′ ITR sequences in an AAV vector need not necessarily be identical or derived from the same AAV serotype or isolate, as long as they function as intended. Generally, ITR sequences allow replication of the vector sequence in the presence of an appropriate mixture of Rep proteins and also allow for the incorporation of the vector sequence into the capsid to generate an AAV vector. In some embodiments, the heterologous nucleic acid is less than about 5 kilobases (kb) in size.

In one embodiment of the fifth aspect, the heterologous nucleic acid comprises a nucleotide sequence encoding a polypeptide. Such a nucleotide sequence may also be referred to as “open reading frame”.

In one embodiment of the fifth aspect, the polypeptide is selected from the group consisting of a neuroprotective polypeptide, an anti-angiogenic polypeptide and a polypeptide that enhances the function of a retinal cell.

In one embodiment of the fifth aspect, the polypeptide is a neuroprotective polypeptide. In another embodiment, the neuroprotective polypeptide is selected from the group consisting of glial derived neurotrophic factor (GDNF), ciliary neurotrophic factor (CNTF), neurotrophin-4 (NT4), nerve growth factor (NGF), brain derived neurotrophic factor (BDNF), and neurturin (NTN).

In one embodiment of the fifth aspect, the polypeptide is an anti-angiogenic polypeptide, wherein the angiogenic polypeptide is preferably selected from the group consisting of a soluble vascular endothelial growth factor (VEGF) receptor, endostatin, tumstatin, angiostatin, a soluble Flt VEGF polypeptide (see e.g., Lai et al. Mol. Ther. 2005; 12:659), a pigment epithelium-derived factor (PEDF), and a soluble Tie-2 receptor. Examples of such anti-angiogenic polypeptide include, but are not limited to, a VEGF-binding antibody, a VEGF-binding fragment thereof, a single chain anti-VEGF antibody and a Fc fusion protein comprising the soluble Flt polypeptide (see e.g., Pechan et al. Gene Ther. 2009; 16:10).

In one embodiment of the fifth aspect, the polypeptide is selected from the group consisting of tissue inhibitor of metalloproteinases-3 (TIMP-3), an opsin including a light-responsive opsin (such as e.g., a rhodopsin or multicharacteristic opsin [MCO], L-opsin, M-opsin or S-opsin), an anti-apoptotic polypeptide (such as e.g., Bcl-2 or Bcl-XI or X-linked inhibitor of apoptosis), fibroblast growth factor 2, epidermal growth factor, Sonic hedgehog, X-linked retinis pigmentosa GTPase regulator (RGPR), retinitis pigmentosa GTPase regulator (RGPR)-interacting protein-1, peripherin-2 (Prph2), MYO7A, Rab escort protein 1 (REP1), lebercilin, retinoschisin including retinoschisin 1 (RS1), Cyclic Nucleotide Gated Channel Subunit Beta 3 (CNGB3), Cyclic Nucleotide Gated Channel Subunit Alpha 3 (CNGA3), a VDM2 protein, isomerase converting all trans retinol ester to 11-cis-retinol (RPE65), phosphodiesterase 6A (PDE6A), phosphodiesterase 6B (PDE6B), phosphodiesterase 6C (PDE6C), retinaldehyde binding protein 1 (RLBP1), nuclear receptor subfamily 2 group E member 3 (NR2E3), MER proto-oncogene tyrosine kinase (MERTK), NADH dehydrogenase subunit 4 (ND4), Raab escort protein-1 (REP1, alternatively referred to as CHM [choroidermia]), RP guanosine triphosphatase regulator (RPGR), complement factor 1 (CF1), Crumbs homolog 1 (CRB1), G-protein subunit alpha transducing 2 (GNAT2), and soluble FMS-like tyrosine kinase 1 (sFLT-1).

In one embodiment of the fifth aspect, the polypeptide is a site-specific nuclease that provides for site-specific knock-down of a gene or correction of a mutation in a gene associated with a retinal disease, wherein the site-specific nuclease is preferably selected from the group consisting of a zinc finger nuclease (ZFN), a transcription activator-like effector nuclease (TALEN), and a CRISPR-Cas protein. Non-limiting examples of Cas proteins include, but are not limited to, CasI, CasIB, Cas2, Cas3, Cas4, Cas5, Cas5e (CasD), Cas6, Cas6e, Cas6f, Cas7, Cas8al, Cas8a2, Cas8b, Cas8c, Cas9 (CsnI or CsxI2), SauriCas9, Cas10, Cas10d, Cas12a, Cas12b, Cas12c, Cas12d, Cas12e, Cas12f, Cas12h, Cas12i, Cas12j, Cas12| (CasBeta), CasMINI, Mad7, CasX, CasY, Cas 13a, CasI4, C2cl, C2c2, C2c3, CasF, CasG, CasH, CsyI, Csy2, Csy3, CseI (CasA), Cse2 (CasB), Cse3 (CasE), Cse4 (CasC), CscI, Csc2, Csa5, Csn2, Csm2, Csm3, Csm4, Csm5, Csm6, Cmr1, Cmr3, Cmr4, Cmr5, Cmr6, CsbI, Csb2, Csb3, CsxI7, CsxI4, CsxIO, CsxI6, CsaX, Csx3, CsxI, CsxI5, Csf1, Csf2, Csf3, Csf4, and Cul966, and homologs or modified versions thereof. The CRISPR-Cas protein may be a CRISPR-Cas protein that generates double stranded breaks (DSB) or a CRISPR-Cas protein that does not generate DSB, such as for example a CRISPR-Cas protein that only cleaves one strand (nickase Cas) or a CRISPR-Cas protein that does not have any nuclease activity (dead Cas). For example, where a dominant allele encodes a defective copy of a gene that when wild-type is a retinal structural protein and/or provides for normal retinal function, a site-specific endonuclease can be targeted to the defective allele and knock out the defective allele. As a further example, in addition to knocking out a defective allele, a site-specific nuclease can also be used to stimulate homologous recombination with a donor DNA that encodes a functional copy of the protein encoded by the defective allele. In the latter example and in one embodiment of the fifth aspect, the AAV vector of the fifth aspect comprises both, a heterologous nucleic acid comprising a nucleotide sequence encoding a site-specific nuclease that knocks out a defective allele and a heterologous nucleic acid corresponding to the functional copy of the defective allele. In other embodiments, the heterologous nucleic acid comprising a nucleotide sequence encoding a site-specific nuclease that knocks out a defective allele and a heterologous nucleic acid corresponding to the functional copy of the defective allele are delivered by two different AAV vectors of the fifth aspect. Such a cargo results in the repair of the defective allele, thereby providing for production of a functional retinal protein (e.g., functional retinoschisin, functional RPE65, functional peripherin or functional CEP290 gene). Site-specific nucleases, in particular the nucleases referred to above, can be engineered to cut specific locations within a genome, and non-homologous end joining can then repair the break while inserting or deleting several nucleotides. In other embodiments of the fifth aspect, the polypeptide is a base editor comprising a CRISPR-Cas protein operably linked to an effector, such as an enzyme or a reverse transcriptase, wherein the CRISPR-Cas system changes a base for another and corrects a mutation in a gene of interest. For example, a CRISPR-Cas protein is fused to an effector (e.g., deaminase) or a CRISPR-Cas system recruits a base editing enzyme (such as a deaminase) to a target DNA sequence through the guide RNA component of the CRISPR-Cas complex protein, that recruits the enzyme through a recruiting RNA motif (such as an aptamer) bound to a gRNA. In other embodiments, the heterologous nucleic acid comprising a nucleotide sequence encoding the base editor may be delivered by two different AAV vectors of the fifth aspect.

Relating to the above paragraph, in one embodiment of the fifth aspect, the AAV vector of the fifth aspect comprises (i) a heterologous nucleic acid comprising a nucleotide sequence encoding a site-specific nuclease that provides for site-specific knock-down of a gene associated with a retinal disease polypeptide, and (ii) a heterologous nucleic acid comprising a functional gene.

In one embodiment of the fifth aspect, the heterologous nucleic acid comprises a nucleotide sequence encoding an RNA or an aptamer, wherein the RNA can be selected from the group consisting of an siRNA, a miRNA and an interfering RNA.

In one embodiment of the fifth aspect, the heterologous nucleic acid encoding a polypeptide, the heterologous nucleic acid encoding an RNA or the heterologous nucleic acid encoding an aptamer is operably linked to a promoter. In some embodiments, the promoter may be a constitutive, inducible and/or tissue-specific promoter. A constitutive promoter can be selected from the group consisting of the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1 promoter. An inducible promoter can be selected from the group consisting of the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system.

In one embodiment of the fifth aspect, the heterologous nucleic acid encoding a polypeptide, an RNA or an aptamer is operably linked to an eye-specific promoter. Non-limiting examples of eye-specific promoters include Ple155 for ON bipolar cells, Ple265 and Ple341 (PCP2) for ON bipolar cells, Ple349 (PDE6H) for cone cells, Ple253 (PITX3) for corneal stroma, Ple32 (CLDN5) for endothelial cells of the blood-retina barrier, Ple316 (NR2E1) for Müller glia, and Ple331 (PAX6) for PAX6 positive cells. See, e.g., Korecki et al. Gene Therapy. 2021; 28, 351-372. Non-limiting examples of eye-specific promoters include GRM6 promoter, GRM6-SV40 promoter and the endogenous murine Grm6 promoter variant. See, e.g., van Wyk et al. Front. Neurosci. 2017; 11:161. Other promoters that can be used are included in Tables 1, 2 and 3 of Buck et al. Int. J. Mol. Sci. 2020; 21, 4197, such as for example, CAG, CB-SB, hRPE65, NA65, CAG, CMV, CAG/CB7, CBA, hVMD2, U6, hGRK1, smCB, hRS1, PR1.7, hCAR, hG1.7, hGRK1, sRLBP1, EF1-alpha, CBh, PGK and others.

In one embodiment of the fifth aspect, the AAV vector comprising any of the variant polypeptides of the disclosure exhibits higher transduction efficiency of the eye compared to the AAV4 wild-type vector (i.e., the AAV4 wild-type vector with the wild-type capsid proteins without an inserted peptide). In one embodiment of the fifth aspect, the AAV vector comprising any of the variant polypeptides of the disclosure exhibits higher transduction efficiency of the eye compared to the AAV5 wild-type vector. In one embodiment of the fifth aspect, the AAV vector comprising any of the variant polypeptides of the disclosure exhibits higher transduction efficiency of the eye compared to the AAV2 wild-type vector. In one embodiment of the fifth aspect, the AAV vector comprising any of the variant polypeptides of the disclosure exhibits higher transduction efficiency of the eye compared to the AAV8 wild-type vector. In these embodiments, the transduction efficiency of the AAV vector of the fifth aspect can be between about 1 to about 500-fold, preferably between about 1 to about 300-fold higher, or between about 2 to about 150-fold higher, or between about 2 to about 100-fold higher, or between 2 to about 50-fold higher, compared to any of the AAV4, AAV5, AAV2 and AAV8 wild-type vectors.

In one embodiment of the fifth aspect, the heterologous nucleic acid comprises a nucleotide sequence encoding more than one polypeptide, such as e.g., at least two subunits of a protein. Multiple polypeptides may be separated by an internal ribozyme entry site (IRES) or a sequence encoding a self-cleaving peptide (e.g., a sequence encoding a 2A peptide) comprised in the heterologous nucleic acid.

In one embodiment of the fifth aspect, the AAV vector comprising any of the variant polypeptides of the disclosure exhibits higher transduction specificity of the eye, in particular in a retina cell selected from the group consisting of bipolar cells (such as ON Rod Bipolar cells, OFF Rod Bipolar cells, ON Cone Bipolar cells, OFF Cone Bipolar cells), retinal ganglion cell (RGC), Mueller Glia cells, Amacrine cells (such as GABA Amacrine cell and Gly Amacrine cells), rod photoreceptors, microglia cells, horizontal cells, cone photoreceptors and vascular cells, compared to the AAV5 wild-type vector (i.e., the AAV5 wild-type vector with the wild-type capsid proteins without an inserted peptide). In one embodiment of the fifth aspect, the AAV vector comprising any of the variant polypeptides of the disclosure exhibits higher transduction specificity of the eye, in particular in a retina cell selected from the group consisting of bipolar cells (such as ON Rod Bipolar cells, OFF Rod Bipolar cells, ON Cone Bipolar cells, OFF Cone Bipolar cells), retinal ganglion cell (RGC), Mueller Glia cells, Amacrine cells (such as GABA Amacrine cell and Gly Amacrine cells), rod photoreceptors, microglia cells, horizontal cells, cone photoreceptors and vascular cells, compared to the AAV2 wild-type vector. In one embodiment of the fifth aspect, the AAV vector comprising any of the variant polypeptides of the disclosure exhibits higher transduction specificity of the eye, in particular in a retina cell selected from the group consisting of bipolar cells (such as ON Rod Bipolar cells, OFF Rod Bipolar cells, ON Cone Bipolar cells, OFF Cone Bipolar cells), retinal ganglion cell (RGC), Mueller Glia cells, Amacrine cells (such as GABA Amacrine cell and Gly Amacrine cells), rod photoreceptors, microglia cells, horizontal cells, cone photoreceptors and vascular cells, compared to the AAV8 wild-type vector. In these embodiments, the transduction specificity of the AAV vector of the fifth aspect can be between about 2 to about 500-fold, preferably between about 2 to about 300-fold higher compared to any of the AAV5, AAV2 and AAV8 wild-type vector.

In one embodiment of the fifth aspect, the AAV vector comprising any of the variant polypeptides of the disclosure exhibits higher transduction specificity of the eye or higher transduction efficiency, in particular in a retina layer selected from the group consisting of nerve fiber layer, ganglion cell layer, inner plexiform layer, inner nuclear layer, outer plexiform layer, outer nuclear layer, photoreceptor layer and retinal pigment epithelium layer (RPE), compared to the AAV5 wild-type vector (i.e., the AAV5 wild-type vector with the wild-type capsid proteins without an inserted peptide). In one embodiment of the fifth aspect, the AAV vector comprising any of the variant polypeptides of the disclosure exhibits higher transduction specificity of the eye or higher transduction efficiency, in particular in a retina layer selected from the group consisting of nerve fiber layer, ganglion cell layer, inner plexiform layer, inner nuclear layer, outer plexiform layer, outer nuclear layer, photoreceptor layer and retinal pigment epithelium layer (RPE), compared to the AAV2 wild-type vector. In one embodiment of the fifth aspect, the AAV vector comprising any of the variant polypeptides of the disclosure exhibits higher transduction specificity of the eye or higher transduction efficiency, in particular in a retina layer selected from the group consisting of nerve fiber layer, ganglion cell layer, inner plexiform layer, inner nuclear layer, outer plexiform layer, outer nuclear layer, photoreceptor layer and retinal pigment epithelium layer (RPE), compared to the AAV8 wild-type vector. In these embodiments, the transduction specificity of the AAV vector of the fifth aspect can be between about 2 to about 500-fold, preferably between about 2 to about 300-fold higher compared to any of the AAV5, AAV2 and AAV8 wild-type vector.

In the sixth aspect, the present disclosure is directed to a pharmaceutical composition comprising the AAV vector according to the fifth aspect (including all embodiments as outlined above) and optionally a pharmaceutically acceptable carrier and/or excipient.

In one embodiment of the sixth aspect, the pharmaceutically acceptable carrier and/or excipient is selected from a buffered saline solution (such as a phosphate buffered saline solution), water, oil (including those of petroleum, animal, vegetable or synthetic origin, such as peanut oil, soybean oil, mineral oil, sesame oil and the like) an emulsion (such as an oil/water emulsion), a wetting agent, and an aqueous dextrose or glycerol solution. In principle, any pharmaceutically acceptable carrier and/or excipient known to the skilled person in connection with an AAV vector may be used. It is preferred that the carrier and/or excipient is sterile.

In the seventh aspect, the present disclosure is directed to a method of delivering a heterologous nucleic acid to a retinal cell, said method comprising contacting the retinal cell with the AAV vector according to the fifth aspect (including all embodiments as outlined above) or the pharmaceutical composition according to the sixth aspect (including all embodiments as outlined above).

In one embodiment of the seventh aspect, the retinal cell is selected from the group consisting of a bipolar cells (such as ON Rod Bipolar cells, OFF Rod Bipolar cells, ON Cone Bipolar cells, OFF Cone Bipolar cells), retinal ganglion cell (RGC), Mueller Glia cells, Amacrine cells (such as GABA Amacrine cell and Gly Amacrine cells), rod photoreceptors, microglia cells, horizontal cells, cone photoreceptors and vascular cells.

In the eighth aspect, the present disclosure is directed to a method of delivering a heterologous nucleic acid to the eye of a subject, said method comprising administering to the subject the AAV vector according to the fifth aspect (including all embodiments as outlined above) or the pharmaceutical composition according to the sixth aspect (including all embodiments as outlined above).

In the ninth aspect, the present disclosure is directed to the AAV vector according to the fifth aspect (including all embodiments as outlined above) or the pharmaceutical composition according to the sixth aspect (including all embodiments as outlined above) for use in preventing or treating an ocular disease.

In an aspect related to the ninth aspect, the present disclosure is directed to a method of preventing or treating an ocular disease comprising administering the AAV vector according to the fifth aspect (including all embodiments as outlined above) or the pharmaceutical composition according to the sixth aspect (including all embodiments as outlined above).

In one embodiment of the ninth aspect and the aspect related to the ninth aspect, the ocular disease is selected from the group consisting of glaucoma, retinitis pigmentosa, macular degeneration including age-related macular degeneration, Leber congenital amaurosis, Leber congenital amaurosis type 10, Usher syndrome 2A, diabetic retinopathy, achromatosis, diabetic macular edema, choroideremia, Leber hereditary optic neuropathy, retinoschisis including X-linked juvenile retinoschisis, inherited retinal dystrophy and color blindness. The skilled person is well aware which of the aforementioned diseases is to be treated with which heterologous nucleic acid comprised in the AAV vector according to the fifth aspect. Such information can e.g., be found in He et al., supra.

In one embodiment of the ninth aspect and the aspect related to the ninth aspect, the AAV vector or the pharmaceutical composition is administered by an injection into the eye. In some embodiments, the injection into the eye is selected from the group consisting of a subretinal injection, an intravitreal injection, a suprachoroidal injection, an intracameral injection and a subconjunctival injection. In some embodiments, the injection into the eye is by a subretinal injection.

In one embodiment of the ninth aspect and the aspect related to the ninth aspect, the AAV vector is administered at about 1×10⁹ to about 1×10¹³ vg/eye, preferably at about 1×10¹⁰ to about 1×10¹² vg/eye, more preferably at about 1.6×10⁹ vg/eye.

In one embodiment of the ninth aspect and the aspect related to the ninth aspect, the AAV vector is administered once or is administered more than once, e.g., from two to four times (in particular, from two to four times daily).

In the tenth aspect, the disclosure relates to a method for enhancing gene transduction in a specific eye cell type comprising administering to the subject the AAV vector according to the fifth aspect of the disclosure (including all embodiments thereof) or the pharmaceutical composition of the sixth aspect (including all embodiments thereof).

In some embodiments, the disclosure relates to a method for enhancing gene transduction in rod photoreceptors comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating a rod-cone dystrophy, a cone-rod dystrophy or rod-related diseases. In some embodiments, the disclosure relates to a method for enhancing gene transduction in rod photoreceptors comprising administering to the subject the AAV vector according to the fifth aspect of the disclosure or the pharmaceutical composition of the sixth aspect of the disclosure, wherein the variant AAV capsid polypeptide comprises a peptide insertion comprising amino acid sequence SEQ ID NO: 2 or SEQ ID NO: 10 or an amino acid sequence having at least 70%, preferably at least 85% sequence identity thereto. In other embodiments, the disclosure relates to an AAV vector comprising a variant AAV capsid polypeptide comprising a peptide insertion comprising amino acid sequence SEQ ID NO: 2 or SEQ ID NO: 10 or an amino acid sequence having at least 70%, preferably at least 85% sequence identity thereto or a pharmaceutical composition comprising said vector for use in preventing or treating a rod-cone dystrophy, a cone-rod dystrophy or rod-related diseases. Non-limiting examples of a rod-cone dystrophy include retinitis pigmentosa, Leber Congenital Amaurosis and Usher Syndrome. Non-limiting examples of a cone-rod dystrophy include Stargardt disease and X-linked retinitis pigmentosa. See, e.g., Hamel (2007). Orphanet Journal of Rare Diseases, 2:7.

In some embodiments, the disclosure relates to a method for enhancing gene transduction in eye cones comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating a rod-cone dystrophy, a cone-rod dystrophy or cone-related diseases. Non-limiting examples of a rod-cone dystrophy include retinitis pigmentosa, Leber Congenital Amaurosis and Usher Syndrome. Non-limiting examples of a cone-rod dystrophy include Stargardt disease and X-linked retinitis pigmentosa. See, e.g., Hamel (2007), supra.

In some embodiments, the disclosure relates to a method for enhancing gene transduction in eye ON rod bipolar cells comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 09 and 17 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 09 and 17 or an amino acid sequence having at least 70%, preferably at least 85% sequence identity thereto or a pharmaceutical composition comprising said vector for use in preventing or treating congenital stationary night blindness or bipolar cells-related diseases. See, e.g., Martemyanov et al. Annu Rev Vis Sci. 2017; 3:25-51. In other embodiments, the disclosure relates to an AAV vector comprising a variant AAV capsid polypeptide comprising a peptide insertion comprising an amino acid sequence selected from the group consisting of SEQ ID NOs: 01 to 09 and 17 or an amino acid sequence having at least 70%, preferably at least 85% sequence identity thereto or a pharmaceutical composition said vector for use in optogenetic tools targeting ON rod bipolar cells. See, e.g., Zhang et al. Front Neurosci. 2022; 16:966772.

In some embodiments, the disclosure relates to a method for enhancing gene transduction in eye ON cone bipolar cells comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating congenital stationary night blindness or bipolar cells-related diseases. See, e.g., Martemyanov et al., supra. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in optogenetic tools targeting ON cone bipolar cells. See, e.g., Zhang et al. Front Neurosci. 2022; 16: 966772.

In some embodiments, the disclosure relates to a method for enhancing gene transduction in eye OFF bipolar cells comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in optogenetic tools targeting OFF bipolar cells. See, e.g., Zhang et al. supra.

In some embodiments, the disclosure relates to a method for enhancing gene transduction in eye Retinal Ganglion cells (RGC) comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in optogenetic tools targeting RGCs. See, e.g., Zhang et al. supra. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating Leber Hereditary Optic Neuropathy, glaucoma or optic neuropathies. See, e.g., Kim et al. Front Neurol. 2021; 12:661938.

In some embodiments, the disclosure relates to a method for enhancing gene transduction in retinal pigment epithelium (RPE) comprising administering to the subject the AAV vector according to the fifth aspect of the disclosure or the pharmaceutical composition of the sixth aspect of the disclosure, wherein the variant AAV capsid polypeptide comprises a peptide insertion comprising an amino acid sequence selected from the group consisting of SEQ ID NOs: 01 to 10 or an amino acid sequence having at least 70%, preferably at least 85% sequence identity thereto. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating a rod-cone dystrophy, a cone-rod dystrophy or cone-related diseases. Non-limiting examples of a rod-cone dystrophy include retinitis pigmentosa, Leber Congenital Amaurosis and Usher Syndrome. Non-limiting examples of a cone-rod dystrophy include Stargardt disease and X-linked retinitis pigmentosa. In other embodiments, the disclosure relates to an AAV vector comprising a variant AAV capsid polypeptide comprising a peptide insertion comprising an amino acid sequence selected from the group consisting of SEQ ID NOs: 01 to 10 or an amino acid sequence having at least 70%, preferably at least 85% sequence identity thereto or a pharmaceutical composition comprising said vector for use in preventing or treating Leber Hereditary Optic Neuropathy, glaucoma or optic neuropathies. See, e.g., Kim et al. Front Neurol. 2021; 12:661938.

### BRIEF DESCRIPTION OF THE FIGURES

FIG. 1 shows the pRepCap plasmid maps for AAV2 (A) and for AAV8 (B), which inter alia served as starting vectors into which the sequences coding for the peptide insertions in Cap2 and Cap8, respectively, were cloned for the generation of the libraries.

FIG. 2 shows the amino acid sequence around the insertions site of the AAV capsid protein of serotypes AAV1, AAV2, AAV3, AAV4, AAV5, AAV6, AAV7, AAV8, AAV9, AAV10, AAV11, AAV12, AAV13, AAVrh10 and AAVrh74, wherein the insertion site is shown as *. The amino acids in bold underlined are the wild-type amino acids, which are amended around the insertion site * as described in Example 1.

FIG. 3 shows the amended amino acids of the wild-type AAV2 starting at position 585 (i.e., G585-Q-R/S, which is R585-G-N in the wild-type AAV2), followed by the peptide insert: 7-mer (top) or 5-mer (bottom) peptides, flanked by a G at the N-terminus and an A at C-terminus, followed by the amended amino acids of the wild-type AAV2 (i.e., Q-A, which is R-Q in the wild-type AAV2). Note that the final A is also present at this position in the wild-type AAV2 sequence. In the nucleotide sequence, W=A or T.

FIGS. 4A and 4B show graphs representing the proportion of unique AAV capsid polypeptide variants for each of the four libraries generated: (A): AAV2, 5mer (_25) and 7mer (_27) before selection (input library) and the proportion of unique variants after the first (“NhpR1”) and second round (“NhpR2”) of selection; (B): AAV8, 5mer (_85) and 7mer (_87) before selection (input library) and the proportion of unique variants after the first (“NhpR1”) and second round (“NhpR2”) of selection.

FIG. 5 shows graphs representing the complexity of the input libraries measured by NGS: the total number of reads, the reads recovered and the different peptides in the libraries.

FIG. 6 shows sequence logos plots representing the amino acid compositions of the four input libraries (amino acids 0-8, wherein amino acid 0 corresponds to S/R at original position 587 (for AAV2) and the 7-mer or 5-mer, respectively, starts at position 2 right after the glycine (G). SEQ ID NO:120 is defined as follows: XGNXXRXXX, wherein X at position 1 is S or R and X at any of positions 4, 5, 7, 8, and 9 is A or R or N or D or C or Q or E or G or H or I or L or Kor Mor F or P or S or T or Y or V or W; SEQ ID NO:121 is defined as follows: XGXXXXXXX, wherein X at position 1 is S or R and X at any of positions 3, 4, 5, 6, 7, 8, and 9 is A or R or N or D or C or Q or E or G or H or I or L or K or M or F or P or S or T or Y or V or W.

FIG. 7 shows an outline of the input and selection rounds in the in vivo experiments of Example 4 (PV=Peptide Variants and Shuffled).

FIG. 8 shows the sequencing results representing the relative AAV abundance of each of the AAV capsid variants (referred here using the internal IDs-Ku numbers) and some benchmarks, such as Ku98, Ku99, Ku100, Ku101, Ku105, Ku106 and Ku108, as compared to the total of reads. CPM=Counts Per Million.

FIG. 9 shows the infection level, indicated as vector genome/diploid genome (vg/dg), in different tissues: ON-target tissues: inferior bleb (inf. Bleb), superior retinal pigment epithelium (RPEa.sup), inferior retinal pigment epithelium (RPEb.inf) and OFF-target tissues: optic nerve retrobulbar (ONR), optical chiasma (OC), lateral geniculate nucleus (LGN) and visual cortex (VC) in the different animals tested (male and female), assessed with qPCR.

FIG. 10 shows the abundance of retinal cell populations (amacrine, cones, horizontal, microglia, Mueller glia, OFF bipolar, ON bipolar, RGC, rods and vascular) in the different tissues (bleb, macula and periphery) and animals (female and male).

FIG. 11 shows the analysis of the average expression (i.e., viral transcripts (barcodes) per AAV) in (A) specific cell populations, as indicated in one of the seven samples evaluated in Example 10 (only Left bleb female sample shown here) and (B) across all cells. CPAM corresponds to the viral transcript counts normalized to endogenous gene expression counts (of that cell), and to Library Input serotype abundance (of that serotype)×106 (scaling factor). The Average Expression is the mean CPAM of the cell population log-normalised (for scaling).

FIG. 12 shows in (A) a heatmap of the abundance of individual AAV capsid variants and benchmark vectors in the retinal pigment epithelium (RPE) tissue. AAV capsid variants are ranked according to cDNA (viral RNA; vRNA) abundance in 8 RPE samples. RPE tissue was collected from the bleb areas, from the superior (A-sup) and inferior blebs (B-inf), from the left (L) and right (R) eye of 1 male NHP (M) and 1 female (F) NHP. Abundance of viral DNA/RNA was quantified by NGS analysis of the AAV-specific barcodes. Example: the top row of the heatmap entitled “vRNA_RPE_A-sup_F_L” shows the viral RNA abundance of the different AAV capsid variants and benchmark vectors in the RPE sample, derived from the superior bleb area in the female left eye. In (B) and (C), boxplots are shown, comprising rankings of the different AAV capsid variants and benchmark vectors according to their vDNA abundance (in (B)) and their vRNA abundance (in (C)) (quantified by NGS analysis of the AAV -specific barcodes) in 8 RPE tissue samples. The ranking is based on the median abundance of the respective AAV capsid variants and benchmark vectors in each of the 8 RPE samples and starts on the left with AAV capsid variant or benchmark vector with the highest abundance followed by AAV capsid variants and benchmark vectors with lower abundance. RPE tissue collection for (B) and (C) as described in (A).

### DETAILED DESCRIPTION OF THE DISCLOSURE

### Definitions

As used herein, the singular form of “a” or “an” also includes the corresponding plural unless the context clearly dictates otherwise.

The term “about” in the context of the present disclosure denotes an interval of accuracy that a person skilled in the art will understand to still ensure the technical effect of the feature in question. The term typically indicates a deviation from the indicated numerical value of +10% and preferably +5%.

It needs to be understood that the term “comprising” is not limiting. For the purposes of the present disclosure, the term “consisting of” is considered to be a preferred embodiment of the term “comprising”. If hereinafter a group is defined to comprise at least a certain number of embodiments, this is also meant to encompass a group which preferably consists of these embodiments only.

The term “variant AAV capsid polypeptide” as used herein refers to a modified or altered form of a wild-type AAV capsid polypeptide, wherein the capsid polypeptide may be a VP1, VP2 or VP3 capsid polypeptide. VP1 wild-type capsid polypeptides of the serotypes AAV1 to AAV13 (including AAV3b), AAVrh10 and AAVrh74 are shown in the sequences of SEQ ID NOs: 30-45. A “variant AAV capsid polypeptide” of the present disclosure contains at least the peptide insertion as defined herein and may contain at least one further insertion, at least one deletion, or at least one substitution of at least one amino acid.

The terms “VP1”, “VP2” and “VP3” are used herein as commonly understood in the art, namely as referring to three transcripts of the AAV cap protein. VP1 is typically the largest protein (in AAV8, the protein consists of 738 amino acids, see SEQ ID NO:38), followed by VP2 (in AAV8, the protein consists of 610 amino acids), followed by the smallest protein VP3 (in AAV8, the protein consists of 535 amino acids).

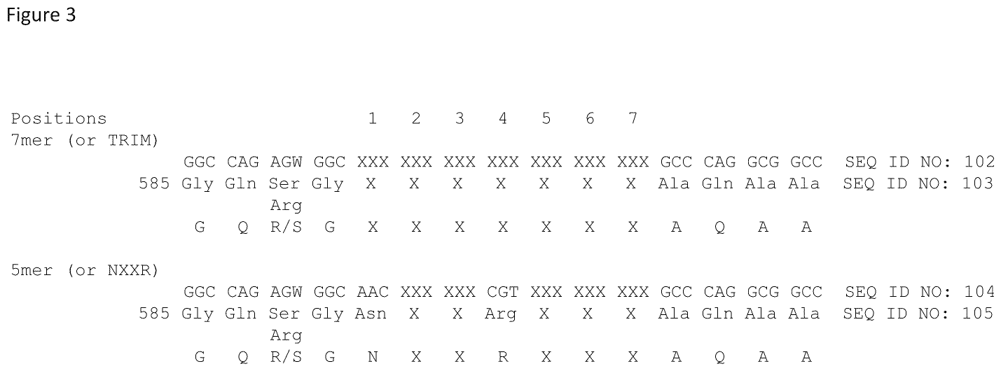

The term “variable region VIII”, as used herein, refers to a stretch of typically about 16 amino acids, which is located between strands βG and βH of the AAV capsid polypeptide and located within the GH loop. The variable region VIII can e.g., be derived from FIG. 3 (shown therein in an alignment of AAV2 and AAV4) of Govindasamy et al. Journal of Virology. 2006; 80 (23): 11556-11570 and from Bōrner et al. Molecular Therapy. 2020; 28 (4): 1016-1032.

The term “variable region IV”, as used herein, refers to a stretch of amino acids located in the β-turn connecting β-sheets GH2 and GH3 of the AAV capsid polypeptide. For AAV2, the variable region IV is located in the β-turn connecting the two β-sheets that build up the highest peak at the three-fold symmetry axis. The variable region IV can e.g., be derived from Boucas et al. supra, Xie et al. supra (alignment between AAV2 and AAV4) and Meyer et al. supra.

The term “sequence identity” is used herein as commonly understood in the art and is defined, with respect to a protein sequence, as the percentage of amino acid or nucleic acid residues in a candidate sequence that are identical to the amino acid or nucleic acid residues in the specific (parental) sequence, after aligning the sequences and introducing gaps, if necessary, to achieve the maximum percent sequence identity, and not considering any conservative substitutions as part of the sequence identity. Techniques for determining a “sequence identity” are known in the field and typically include providing a nucleotide sequence or an amino acid sequence and comparing such a sequence to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequence and multiplied by 100. Alignment for purposes of determining percent sequence identity can be achieved in various ways that are within the skill in the art, for instance, using publicly available computer software such as BLAST, BLAST-2, ALIGN or Megalign (DNASTAR) software. Percent identity may be determined, for example, by comparing sequence information using the advanced BLAST computer program, including version 2.15.0, available from the National Institutes of Health. The BLAST program is based on the alignment method of Karlin and Altschul (1990) Proc. Natl. Acad. Sci. USA 87:2264-2268 and as discussed in Altschul et al. J. Mol. Biol. 1990; 215:403-410; Karlin and Altschul. Proc. Natl. Acad. Sci. USA. 1993; 90:5873-5877; and Altschul et al. Nucleic Acids Res. 1997; 25:3389-3402. Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen. Comput. and Chem. 1993; 17:149-163. Those skilled in the art can determine appropriate parameters for measuring alignment, including any algorithms needed to achieve maximal alignment over the full length of the sequences being compared.

The term “polypeptide” as used herein refers to a compound comprised of amino acid residues covalently linked by peptide bonds and is used interchangeably with the terms “protein” and “peptide”. A polypeptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The term “residue,” as used herein, refers to a position in a protein and its associated amino acid identity. For example, Asparagine 587 (also referred to as Asn587 or N587) is a residue at position 587 in a specific protein.

Whenever a specific amino acid is referred to herein, for example (i) G or A that is (additionally) comprised in a peptide sequence or (ii) G, Q, R, S or A that is (alone or in combination with others, for example GQR/S or QAA) resulting from a change of one (or more) underlying amino acid(s) in a protein sequence (such as a wild-type capsid protein), it is understood that this specific amino acid may be replaced by an amino acid from the same class, which can be referred to as a conservative amino acid replacement. In other words, a reference to a specific amino acid is understood as referring to the class, to which this amino acid belongs, and includes any of the further members of this class, with the classes being as follows: (i) aliphatic amino acids: G, A, V, L and I; (ii) hydroxyl- or sulfur-containing amino acids: S, C, T and M; (iii) aromatic amino acids: F, Y and W; (iv) basic amino acids: H, K and R; (v) acidic amino acids: D and E; as well as (vi) amides thereof: N and Q. Thus, if reference is, for example, made to G, the amino acid at this position may alternatively be A, V, L or I. If reference is made, for example, to A, the amino acid at this position may alternatively be G, V, L or I. If reference is made, for example, to Q, the amino acid at this position may alternatively be N. If reference is made, for example, to R, the amino acid at this position may alternatively be H or K. If reference is made, for example, to S, the amino acid at this position may alternatively be C, T or M.

The term “isolated” as used herein means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell or it can be present in a host cell that has been genetically modified to express the isolated nucleic acid. An isolated cell refers to a cell separated from the subject organism.

The term “nucleic acid” as used herein is defined as a chain of nucleotides and interchangeably used with the term “polynucleotide”. A skilled person has the general knowledge that nucleic acids are polynucleotides, which can be hydrolyzed into the monomeric “nucleotides”. The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means. The term “polynucleotide” as used herein is to be interpreted broadly, and includes DNA and RNA, including single-stranded (ss) DNA or RNA as well as double-stranded (ds) DNA or RNA and further including chemically modified DNA and RNA.

The term “DNA” as used herein is the usual abbreviation for deoxyribonucleic acid. It is a nucleic acid molecule, i.e., a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e., deoxyribose, of a first and a phosphate moiety of a second, adjacent monomer. The specific order of the monomers, i.e., the order of the bases linked to the sugar/phosphate-backbone, is called the DNA-sequence. DNA may be single stranded or double stranded. In the double stranded form, the nucleotides of the first strand typically hybridize with the nucleotides of the second strand, e.g., by A/T-base-pairing and G/C-base-pairing.

The term “RNA” as used herein relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a p-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to the addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA. In one embodiment, the RNA may have modified ribonucleotides. Examples of modified ribonucleotides include, without limitation, 5-methylcytidine, pseudouridine and/or 1-methyl-pseudouridine. In some embodiments, the RNA comprises a modified nucleoside in place of at least one (e.g., every) uridine. In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′ to 5′ triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand or may be attached to RNA post-transcriptionally using capping enzymes.

The term “mRNA” as used herein relates to an RNA transcript which encodes a peptide or protein. As established in the field, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

The term “chemically modified” with respect to a nucleic acid or DNA or RNA, respectively, as used herein refers to chemically modified nucleotides, such as e.g., the above-mentioned ribonucleotides in the RNA definition. The term also includes any chemical modifications in the phosphate-groups linking the nucleotides.

The term “open reading frame” as used herein refers to a specific sequence of nucleotides comprised in a nucleic acid that typically starts with a start codon (typically an ATG) and ends with a stop codon (typically a TAA, TAG or TGA), which “encodes” a subject matter as defined next.

The term “encodes” or “encoding” as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA, mRNA, interfering RNA or miRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In the context of the present disclosure, the term “transcription” relates to a process, wherein the genetic code in a DNA sequence is transcribed into RNA. Subsequently, the RNA may be translated into peptide or protein.

The term “translation” relates to the process in the ribosomes of a cell by which a strand of mRNA directs the assembly of a sequence of amino acids to make a peptide or protein.

The term “expressed” or related terms such as “expression” as used herein is the transcription and/or translation of a particular nucleotide sequence such that the final product, such as in particular a protein or an RNA, in particular an interfering RNA, is produced.

The term “operably linked” as used herein refers to functional linkage between a regulatory sequence and a second nucleic acid sequence, typically an open reading frame, resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to an open reading frame (or coding sequence) if the promoter affects the transcription or expression of the open reading frame (or coding sequence). Generally, operably linked DNA sequences are contiguous and, where necessary to join two protein coding regions, in the same reading frame.

The term “promoter” as used herein is defined as a DNA sequence recognized by the synthetic machinery of the cell, or introduced synthetic machinery, required to initiate the specific transcription of a polynucleotide sequence. The term “enhancer” as used herein relates to an element that enhances the activity of the promoter. The term “functional fragment thereof” as used herein means that only a part of an endogenous promoter, optionally in combination with an enhancer, is present, which is nevertheless functional in that it acts as promoter.

The term “AAV vector” as used herein refers to an AAV capsid which comprises an isolated nucleic acid and which can be used to deliver the isolated nucleic acid to the interior of a cell (by way of transduction as defined below) such that the isolated nucleic acid comprised in the AAV capsid is expressed inside the cell. An AAV vector can be used to generate recombinant AAV particles by using a host cell.

The term “AAV capsid” as used herein refers to a capsid that is typically formed by the AAV proteins VP1, VP2 and VP3 as defined above, wherein typically 60 of these proteins assemble in a stochiometric ratio of about 1:1:10 (VP1:VP2:VP3) arranged in T=1 icosahedral symmetry.

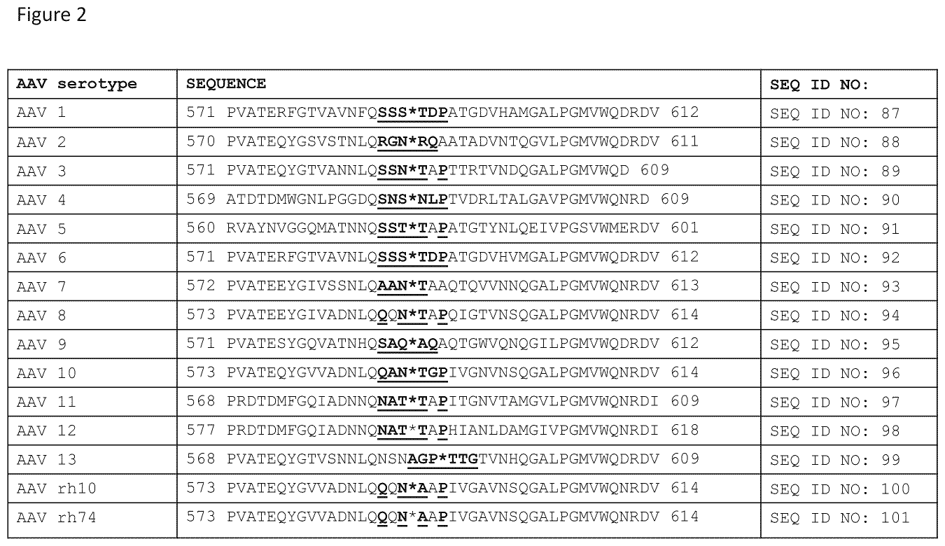

The term “transduction” as used herein refers to the process of delivering an isolated nucleic acid into a cell using an AAV vector. A typical way and route of AAV transduction is exemplary shown in FIG. 2C of He et al., supra. Transduction can occur in vitro or in vivo.

The term “transduction efficiency” as used herein refers to the percentage of target cells or proportion of target tissue transduced with at least one copy of an AAV vector, i.e., the percentage of cells wherein the AAV vector has been introduced. Transduction efficiency of the AAV vector of the present disclosure may be determined by measuring the level of expression per cell, i.e., the amount of protein or mRNA expression, as quantified after introduction of an AAV vector on target cells or tissue. This can be measured, for example, by using scRNAseq experiments.

The term “higher transduction efficiency”, as used herein, refers to the ability of an AAV vector to be introduced at a higher percentage of target cells or a higher proportion of target tissue as compared to a control vector. Furthermore, a higher transduction efficiency can be defined as the level of mRNA or protein expression in target cells or tissue with a value above 1 when normalized relative to a wild-type AAV set to 1.

The term “transduction specificity”, as used herein, refers to the ability of an AAV vector to preferentially transduce a particular cell or tissue type over other cell types, wherein the AAV vector of the present disclosure has a transduction specificity for the eye, in particular for any of the following cells of the retina: bipolar cells (such as ON Rod Bipolar cells, OFF Rod Bipolar cells, ON Cone Bipolar cells, OFF Cone Bipolar cells), retinal ganglion cell (RGC), Mueller Glia cells, Amacrine cells (such as GABA Amacrine cell and Gly Amacrine cells), rod photoreceptors, microglia cells, horizontal cells, cone photoreceptors and vascular cells. Transduction specificity may be determined by the presence of transgene protein expression in the eye after the application or administration of an AAV vector of the present application.

The term “higher transduction specificity” refers to increased or enhanced ability of an AAV vector to preferentially target and transduce a particular cell or tissue type as compared to a control vector. A higher transduction specificity can be defined as the relative number of transgene protein expression in the target cells or tissue in comparison with all other tissues with a value above 1 when normalized relative to wild-type AAV set to 1.

The term “heterologous” as used herein refers to a nucleic acid derived from a genotypically distinct entity from that of the rest of the entity to which it is being compared. For example, a nucleic acid introduced by genetic engineering techniques into a plasmid or vector derived from a different species is a heterologous nucleic acid. A promoter removed from its native coding sequence and operatively linked to a coding sequence with which it is not naturally found linked is a heterologous promoter. An AAV vector that includes a heterologous nucleic acid encoding a heterologous gene product is an AAV vector that includes a nucleic acid not normally included in a naturally-occurring, wild-type AAV, and the encoded heterologous gene product is a gene product not normally encoded by a naturally-occurring, wild-type AAV.

As used herein, the term “pharmaceutically acceptable” as used in connection with a composition of the disclosure refers to molecular entities and other ingredients of such compositions that are physiologically tolerable and do not produce undesired reactions when administered to a mammal (e.g., human). The term “pharmaceutically acceptable” can also mean approved by a regulatory agency of the Federal or a state government or listed in the U.S. Pharmacopeia or other generally recognized pharmacopeia for use in mammals, and more particularly in humans. A “pharmaceutically acceptable carrier and/or excipient” refers to an ingredient in a pharmaceutical composition or formulation, other than an active ingredient, which is nontoxic to a subject.

The term “subject” as used herein refers to a human or another mammal (e.g., mouse, rat, rabbit, dog, cat, cattle, swine, sheep, horse or primate) that can be afflicted with or is susceptible to a disease or disorder (in particular an eye disease or disorder) but may or may not have the disease or disorder. In many embodiments, the subject is a human being. Unless otherwise stated, the term “subject” does not denote a particular age, and thus encompass adults, elderlies, children, and newborns. In embodiments, the “subject” is a “patient”, e.g., a human patient. The term “patient” means a subject for treatment, in particular a diseased subject.

The term “treatment” or “treating” as used herein relates to the management and care of a subject for the purpose of combating a condition such as a disease or disorder. The term is intended to include the full spectrum of treatments for a given condition from which the subject is suffering, such as administration of a therapeutically effective composition to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The present application also relates to the following embodiment list:

1. A variant adeno-associated virus (AAV) capsid polypeptide comprising a peptide insertion in the variable region IV or in the variable region VIII relative to a wild-type AAV capsid polypeptide, wherein the peptide insertion comprises an amino acid sequence selected from the group consisting of SEQ ID NOs: 1-29 or an amino acid sequence having at least 70%, preferably at least 85% sequence identity thereto.

2. The variant polypeptide according to embodiment 1, wherein the peptide insertion comprises an amino acid sequence selected from the group consisting of SEQ ID NOs: 1-10 or an amino acid sequence having at least 85% sequence identity thereto.

3. The variant polypeptide according to embodiment 1 or 2, wherein the peptide insertion further comprises a G at the N-terminus and/or an A at the C-terminus.

4. The variant polypeptide according to any one of embodiments 1 to 3, wherein the peptide insertion consists of an amino acid sequence of a G at the N-terminus, followed by an amino acid sequence selected from the group consisting of SEQ ID Nos: 1-10, followed by an A at the C-terminus, or an amino acid sequence having at least 88% sequence identity thereto.

5. The variant polypeptide according to any one of embodiments 1 to 4, wherein the AAV is AAV1 and the peptide insertion is immediately after an amino acid selected from amino acids 580 to 595 (VP1 numbering) of AAV1, wherein the AAV is AAV2 and the peptide insertion is immediately after an amino acid selected from amino acids 579 to 594 (VP1 numbering) of AAV2, wherein the AAV is AAV3 and the peptide insertion is immediately after an amino acid selected from amino acids 580 to 595 (VP1 numbering) of AAV3, wherein the AAV is AAV3b and the peptide insertion is immediately after an amino acid selected from amino acids 580 to 595 (VP1 numbering) of AAV3b, wherein the AAV is AAV4 and the peptide insertion is immediately after an amino acid selected from amino acids 578 to 593 (VP1 numbering) of AAV4, wherein the AAV is AAV5 and the peptide insertion is immediately after an amino acid selected from amino acids 569 to 584 (VP1 numbering) of AAV5, wherein the AAV is AAV6 and the peptide insertion is immediately after an amino acid selected from amino acids 580 to 595 (VP1 numbering) of AAV6, wherein the AAV is AAV7 and the peptide insertion is immediately after an amino acid selected from amino acids 581 to 596 (VP1 numbering) of AAV7, wherein the AAV is AAV8 and the peptide insertion is immediately after an amino acid selected from amino acids 582 to 597 (VP1 numbering) of AAV8, wherein the AAV is AAV9 and the peptide insertion is immediately after an amino acid selected from amino acids 580 to 595 (VP1 numbering) of AAV9, wherein the AAV is AAV10 and the peptide insertion is immediately after an amino acid selected from amino acids 582 to 597 (VP1 numbering) of AAV10, wherein the AAV is AAV11 and the peptide insertion is immediately after an amino acid selected from amino acids 575 to 593 (VP1 numbering) of AAV11, wherein the AAV is AAV12 and the peptide insertion is immediately after an amino acid selected from amino acids 584 to 600 (VP1 numbering) of AAV12, wherein the AAV is AAV13 and the peptide insertion is immediately after an amino acid selected from amino acids 575 to 593 (VP1 numbering) of AAV13, wherein the AAV is AAVrh10 and the peptide insertion is immediately after an amino acid selected from amino acids 582 to 597 (VP1 numbering) of AAV10rh10, or wherein the AAV is AAVrh74 and the peptide insertion is immediately after an amino acid selected from amino acids 582 to 597 (VP1 numbering) of AAVrh74.

6. The variant polypeptide according to any one of embodiments 1 to 5, wherein the AAV is AAV1 and the AAV1 capsid polypeptide excluding the peptide insertion comprises or consists of the wild-type capsid amino acid sequence of AAV1 of SEQ ID NO:30 or an amino acid sequence having at least 80% sequence identity thereto, (b) the AAV is AAV2 and the AAV2 capsid polypeptide excluding the peptide insertion comprises or consists of the wild-type capsid amino acid sequence of AAV2 of SEQ ID NO:31 or an amino acid sequence having at least 80% sequence identity thereto, (c) the AAV is AAV3 and the AAV3 capsid polypeptide excluding the peptide insertion comprises or consists of the wild-type capsid amino acid sequence of AAV3 of SEQ ID NO:32 or an amino acid sequence having at least 80% sequence identity thereto, (d) the AAV is AAV3b and the AAV3b capsid polypeptide excluding the peptide insertion comprises or consists of the wild-type capsid amino acid sequence of AAV3b of SEQ ID NO:33 or an amino acid sequence having at least 80% sequence identity thereto, (e) the AAV is AAV4 and the AAV4 capsid polypeptide excluding the peptide insertion comprises or consists of the wild-type capsid amino acid sequence of AAV4 of SEQ ID NO:34 or an amino acid sequence having at least 80% sequence identity thereto, (f) the AAV is AAV5 and the AAV5 capsid polypeptide excluding the peptide insertion comprises or consists of the wild-type capsid amino acid sequence of AAV5 of SEQ ID NO:35 or an amino acid sequence having at least 80% sequence identity thereto, (g) the AAV is AAV6 and the AAV6 capsid polypeptide excluding the peptide insertion comprises or consists of the wild-type capsid amino acid sequence of AAV6 of SEQ ID NO:36 or an amino acid sequence having at least 80% sequence identity thereto, (h) the AAV is AAV7 and the AAV7 capsid polypeptide excluding the peptide insertion comprises or consists of the wild-type capsid amino acid sequence of AAV7 of SEQ ID NO:37 or an amino acid sequence having at least 80% sequence identity thereto, (i) the AAV is AAV8 and the AAV8 capsid polypeptide excluding the peptide insertion comprises or consists of the wild-type capsid amino acid sequence of AAV8 of SEQ ID NO:38 or an amino acid sequence having at least 80% sequence identity thereto, (j) the AAV is AAV9 and the AAV9 capsid polypeptide excluding the peptide insertion comprises or consists of the wild-type capsid amino acid sequence of AAV9 of SEQ ID NO:39 or an amino acid sequence having at least 80% sequence identity thereto, (k) the AAV is AAV10 and the AAV10 capsid polypeptide excluding the peptide insertion comprises or consists of the wild-type capsid amino acid sequence of AAV10 of SEQ ID NO:40 or an amino acid sequence having at least 80% sequence identity thereto, (I) the AAV is AAV11 and the AAV11 capsid polypeptide excluding the peptide insertion comprises or consists of the wild-type capsid amino acid sequence of AAV11 of SEQ ID NO:41 or an amino acid sequence having at least 80% sequence identity thereto, (m) the AAV is AAV12 and the AAV12 capsid polypeptide excluding the peptide insertion comprises or consists of the wild-type capsid amino acid sequence of AAV12 of SEQ ID NO:42 or an amino acid sequence having at least 80% sequence identity thereto, (n) the AAV is AAV13 and the AAV13 capsid polypeptide excluding the peptide insertion comprises or consists of the wild-type capsid amino acid sequence of AAV13 of SEQ ID NO: 43 or an amino acid sequence having at least 80% sequence identity thereto, (o) the AAV is AAVrh10 and the AAVrh10 capsid polypeptide excluding the peptide insertion comprises or consists of the wild-type capsid amino acid sequence of AAVrh10 of SEQ ID NO:44 or an amino acid sequence having at least 80% sequence identity thereto, or (p) the AAV is AAVrh74 and the AAVrh74 capsid polypeptide excluding the peptide insertion comprises or consists of the wild-type capsid amino acid sequence of AAVrh74 of SEQ ID NO:45 or an amino acid sequence having at least 80% sequence identity thereto.

7. An isolated nucleic acid encoding the variant polypeptide according to any one of embodiments 1 to 6.

8. A recombinant polynucleotide comprising the nucleic acid according to embodiment 7.

9. An isolated cell comprising the polypeptide of any one of embodiments 1 to 6, the nucleic acid according to embodiment 7 or the recombinant polynucleotide according to embodiment 8.

10. An adeno-associated virus (AAV) vector comprising the variant polypeptide according to any one of embodiments 1 to 6, wherein the AAV vector preferably further comprises a heterologous nucleic acid.

11. The AAV vector of embodiment 10, wherein the heterologous nucleic acid comprises a nucleotide sequence encoding (i) a polypeptide selected from the group consisting of tissue inhibitor of metalloproteinases-3 (TIMP-3), an opsin, an anti-apoptotic polypeptide, fibroblast growth factor 2, epidermal growth factor, Sonic hedgehog, X-linked retinis pigmentosa GTPase regulator (RGPR), retinitis pigmentosa GTPase regulator (RGPR)-interacting protein-1, peripherin-2 (Prph2), MYO7A, Rab escort protein 1 (REP1), lebercilin, retinoschisin, Cyclic Nucleotide Gated Channel Subunit Beta 3 (CNGB3), Cyclic Nucleotide Gated Channel Subunit Alpha 3 (CNGA3), a VDM2 protein, isomerase converting all trans retinol ester to 11-cis-retinol (RPE65), phosphodiesterase 6A (PDE6A), phosphodiesterase 6B (PDE6B). phosphodiesterase 6C (PDE6C), retinaldehyde binding protein 1 (RLBP1), nuclear receptor subfamily 2 group E member 3 (NR2E3), MER proto-oncogene tyrosine kinase (MERTK), NADH dehydrogenase subunit 4 (ND4), Raab escort protein-1 (REP1), RP guanosine triphosphatase regulator (RPGR), complement factor 1 (CF1), Crumbs homolog 1 (CRB1), G-protein subunit alpha transducing 2 (GNAT2), soluble FMS-like tyrosine kinase 1 (sFLT-1), a neuroprotective polypeptide, an angiogenic polypeptide and a site-specific nuclease; (ii) an interfering RNA, (iii) a CRISPR-Cas protein or (iv) an aptamer.

12. A pharmaceutical composition comprising the AAV vector according to embodiment 10 or 11 and optionally a pharmaceutically acceptable carrier and/or excipient.

13. A method of delivering a heterologous nucleic acid to a retinal cell, said method comprising contacting the retinal cell with the AAV vector according to embodiment 10 or 11 or the pharmaceutical composition according to embodiment 12.

14. A method of delivering a heterologous nucleic acid to the eye of a subject, the method comprising administering to the subject the AAV vector according to embodiment 10 or 11 or the pharmaceutical composition according to embodiment 12.

15. The AAV vector according to embodiment 10 or 11 or the pharmaceutical composition according to embodiment 12 for use in preventing or treating an ocular disease, wherein the ocular disease is preferably selected from the group consisting of glaucoma, retinitis pigmentosa, macular degeneration including age-related macular degeneration, Leber congenital amaurosis, Leber congenital amaurosis type 10, Usher syndrome 2A, diabetic retinopathy, achromatosis, diabetic macular edema, choroideremia, Leber hereditary optic neuropathy, retinoschisis including X-linked juvenile retinoschisis and color blindness.

Embodiments of inventive compositions and methods are illustrated in the following examples. These examples are provided for illustrative purposes and are not considered limitations on the scope of inventive compositions and methods.

### EXAMPLES

### Example 1: Generation of Peptide Insertion Libraries

Engineered recombinant AAV (rAAV) libraries of three types were generated: (i) peptide display on AAV2 capsid, (ii) peptide display on AAV8 capsid and (iii) AAV shuffled libraries. The AAV shuffled comprised (a) AAV1 to 9+rh10, (b) AAV1 to 3+AAV 6 to 9+rh10 and (c) CapB1, CapDJ, CapLK03, CapM41, CapSH10 and CapAnc80.

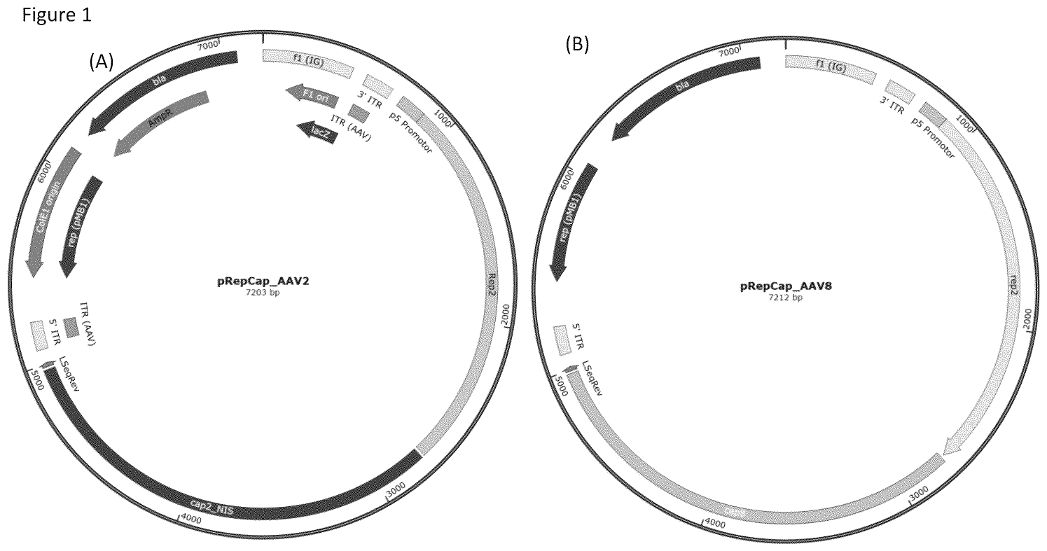

For the generation of the libraries, plasmids for AAV2 peptide display, AAV8 peptide display and the shuffled AAVs were used, in which the viral genome (Rep2Cap2 or Cap8 or shuffled) is flanked by ITRs (Boerner K et al. Mol Ther. 2020; 28:1016-1032) (FIG. 1 shows the plasmids for AAV2 and AAV8).

The method is now described for the AAV2 and AAV8 libraries: A peptide insertion site (PIS) was cloned immediately after VP1 positions nt1752/aa584 (nucleotide/amino acid) for AAV2 (the PIS starting with R585) and immediately after nt1761/aa587 for AAV8 (the PIS starting with Q588), see FIG. 2 for the wt AAV2 and AAV8 as well as further serotype sequences, wherein the position as indicated (e.g. 571 for AAV1) is the position of the first mentioned amino acid (e.g. the “P” for AAV1). The first peptide display position starts after amino acid 587 (N587 in the original sequence is converted into R/S587 upon peptide insertion) for AAV2 and after amino acid 590 (N590 in the original sequence is converted to R/S590 upon peptide insertion) for AAV8, respectively. To facilitate the cloning of the DNA sequence of the peptide insertion, the DNA sequence before and after the insertion site was changed in order to introduce restriction sites, which resulted, if applicable, in changes in the corresponding amino-acid positions. The 3 amino acid positions before the peptide insertion (RGN in the AAV2 wt, as well as QQN in the AAV8 wt) were changed to glycine, glutamine and arginine or serine (GQR/S). The peptide was flanked by a glycine and an alanine, which are fixed amino acids in the peptide insert. The inserted peptide is followed by three amino acids-glycine, alanine and alanine (QAA), wherein the QAA sequence is not part of the inserted peptide but the wt sequences have been changed accordingly, if applicable, i.e., RQ for AAV2 was changed to QA (the next amino acid being an A already in wt) and TAP for AAV8 was changed to QAA. Therefore, the peptide display consisted of G followed by seven either fully randomized positions (referred to as AAV2_7mer or AAV8_7mer) or N at position 1, R at position 4 and fully randomized in the rest of the 7 positions (referred to as AAV2_5mer or AAV8_5mer), followed by A (FIG. 3). Thus, overall, the inserted peptide had 9 amino acids, of which only 7 or 5 positions are randomized. It was flanked on the N-terminus by GQR or GQS and on the C-terminus by QAA.

The DNA sequences to be inserted into the plasmids were purchased from Ella Biotech, which used 2′-deoxynucleoside trimer phosphoramidites, allowing the addition of nucleotides in trimers. This in turn allowed the selection of 20 codons to represent the 20 amino acids (Table 1), thereby reducing the final complexity of the library on the DNA level. The DNA oligonucleotides were ordered and, using a reverse primer and a polymerase, were converted to a double stranded DNA (dsDNA). The dsDNA oligonucleotide and the plasmid vectors (see FIG. 1 for the AAV2 and AAV8 vectors) were digested with BglI and SfiI respectively (Boerner K et al., supra), ligated overnight and electroporated into E. cloni® 10G ELITE cells.

TABLE 1
Codons used for peptide display oligonucleotides

| | | Codon XXX | |
|---|---|---|---|
| Lys | AAA | 5.00 | K |
| Asn | AAC | 5.00 | N |
| Thr | ACC | 5.00 | T |
| Ile | ATC | 5.00 | I |
| Met | ATG | 5.00 | M |
| Gln | CAG | 5.00 | Q |
| His | CAT | 5.00 | H |
| Pro | CCA | 5.00 | P |
| Arg | CGT | 5.00 | R |
| Leu | CTG | 5.00 | L |
| Glu | GAA | 5.00 | E |
| Asp | GAT | 5.00 | D |
| Ala | GCA | 5.00 | A |
| Gly | GGT | 5.00 | G |
| Val | GTT | 5.00 | V |
| Tyr | TAC | 5.00 | Y |
| Ser | TCT | 5.00 | S |
| Cys | TGC | 5.00 | C |
| Trp | TGG | 5.00 | W |
| Phe | TTC | 5.00 | F |

After transformation, a small aliquot of the transformation reaction was plated onto agar plates to calculate the transformation efficiency and the rest was used to inoculate a bacterial culture, which was then used for the plasmid library DNA preparation. Approximately 10-20 bacterial colonies per library were used for Sanger sequencing, as a first quality control step.

The library diversity was calculated as maximum potential diversity based on the number of transformants; that is bacterial colonies. Since not all transformants are unique, the number of transformants does not represent the actual library diversity-however, it is the upper limit of library diversity. Due to technological limitations, such as the transformation efficiency of the competent cells or the efficiency of transformation (an upper limit of about 1×10⁸), the maximum potential library diversity for the 7mer libraries was lower than its maximum theoretical diversity, whereas the 5mer libraries could reach their maximum theoretical diversity, based on the sequence complexity:

$$5 mer 2 \times {10}^{6} = 3.2 \times {10}^{6} \times 2 ( R / S at position - 2 ) = 6.4 \times {10}^{6} unique sequences$$

$$7 mer 2 \times {10}^{8} = 1.28 \times {10}^{9} \times 2 ( R / S at position - 2 ) = 2.56 \times {10}^{9} unique sequences$$

The maximum theoretical diversity could also be limited by the quality and/or diversity of the purchased single stranded oligonucleotides and/or the reverse strand polymerase reaction.

The maximum potential diversity of the libraries is shown in Table 2, together with the estimated percent of plasmid clones without an insertion and the fold coverage of the maximum theoretical diversity.

TABLE 2
Maximum theoretical and potential diversity, estimated percentage
of capsids without insertion and coverage of the peptide libraries.

| | | Maximum | Estimated % | |
|---|---|---|---|---|
| | Maximum | potential | of capsids | |
| Plasmid | theoretical | diversity | without | |
| Library | diversity | (transformants) | insertion | Coverage |
| AAV2_5mer | 6.4 × 10⁶ | 4.2 × 10⁷ | 14% | 5.64 fold |
| AAV8_5mer | 6.4 × 10⁶ | 2.7 × 10⁷ | 2.9% | 4.096 fold |
| AAV2_7mer | 2.56 × 10⁹ | 7.9 × 10⁷ | 3.2% | 0.030 fold |
| AAV8_7mer | 2.56 × 10⁹ | 1.3 × 10⁸ | 10.5% | 0.045 fold |

### Example 2: AAV Vector Production of Peptide Insertion Libraries

The generated plasmid libraries together with a helper plasmid were used to produce the AAV libraries. The molecular ratio between the RepCap viral genome (library plasmid) and the helper plasmids was 1:20, so that the total number of plasmid copies per cell was about 10000 for the RepCap, in order to avoid cross-packaging, as shown previously (Körbelin et al. Gene Ther. 2017; 24:470-481 & Körbelin et al., Mol Ther. 2016; 24:1050-1061). Each library was produced in small scale and the total yield was between 2×10¹¹-1×10¹² viral genomes (vg), quantified by ddPCR.

### EXAMPLE 3: INPUT AAV LIBRARY COMPLEXITY

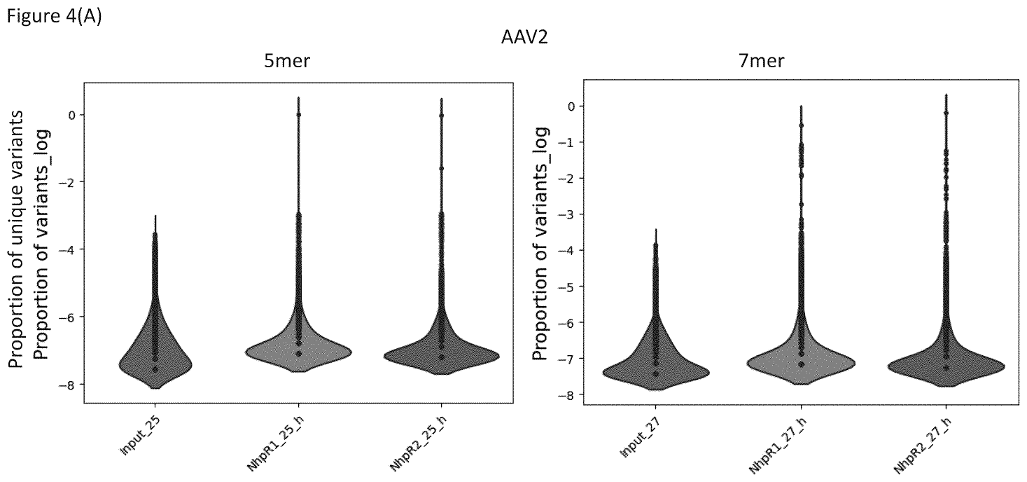

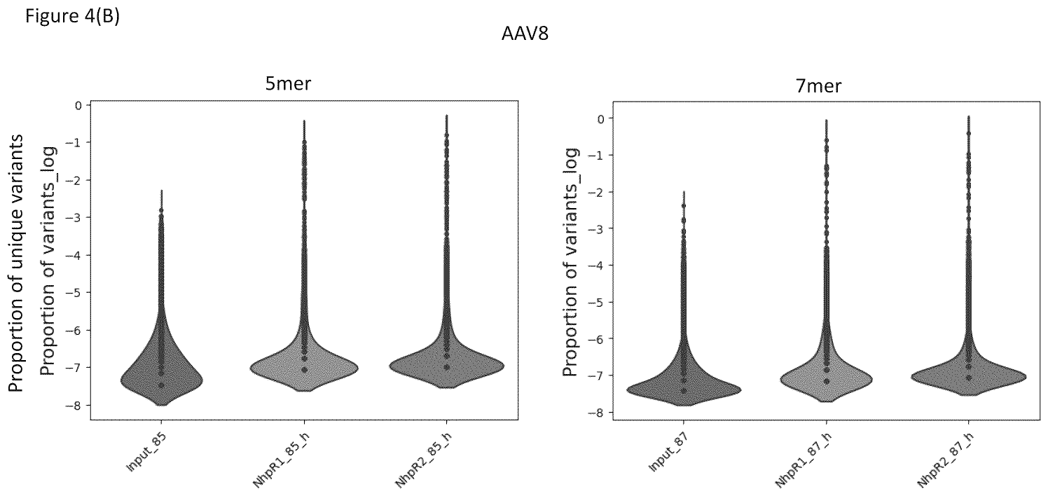

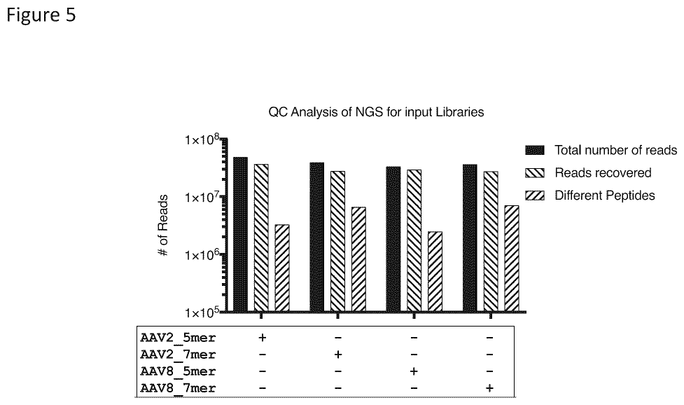

After AAV production, the PIS was amplified by PCR and subjected to amplicon next generation sequencing (NGS) (Rapti K et al. J Vis Exp. 2022; 188). As shown in FIG. 5, all 4 AAV2 and AAV8 libraries showed high complexity, about 1/1×10⁷. The NGS methodology used allows a maximum number of reads of about 1×10⁷ per library. This covers the 5mer library complexity, but it is lower than what is needed for the 7mer libraries. If each library contained only unique variants, each peptide variant in the 5mer would have a proportion of 1/6.4×10⁶ (this is the maximum theoretical complexity of the library, which is covered by the number of bacterial colonies) and in the 7mer about 1/1×10⁸ (this is the number of bacterial colonies, that does not cover the maximum theoretical diversity of 2.56×10⁹). Considering we only have 1×10⁷ reads in NGS, we can only read 10% of the 7mer library, which explains the median proportion seen in the graph. Accordingly, the number of unique variants in the libraries identified by NGS were approximately 3×10⁶ for the 5mer libraries and about 7×10⁶ for the 7mer libraries (FIG. 4).

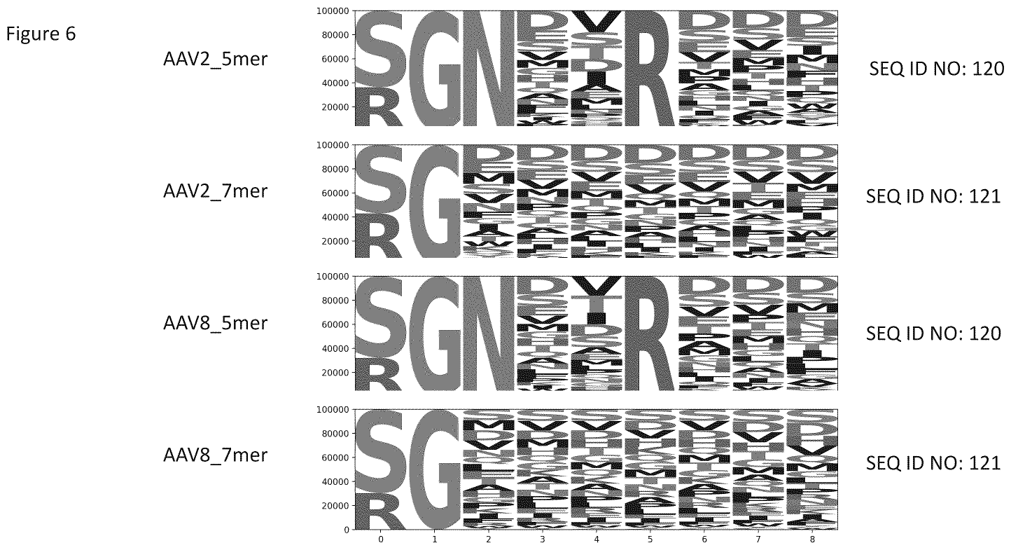

In agreement to the proportion analysis, the 5mer libraries seemed to cover the maximal diversity, whereas the 7mer libraries were below the limit, which however could be due to the cloning and sequencing sensitivity limitations. Finally, as shown in FIG. 6, sequence logos were generated from the top 1×10⁵ variants of each library. The libraries showed moderately balanced representation of all amino acids in each position, with the exception of positions 1 and 4 in the 5mer libraries, where N and R respectively dominate, as expected.

For the shuffled libraries, after AAV production, the cap gene of each of the 3 shuffled libraries was PCR amplified, cloned to a new vector (without ITRs) and 10 colonies from each library, a total of 30, were sent for Sanger sequencing. Each clone was unique, suggesting high variability.

### Example 4: In Vivo Selection

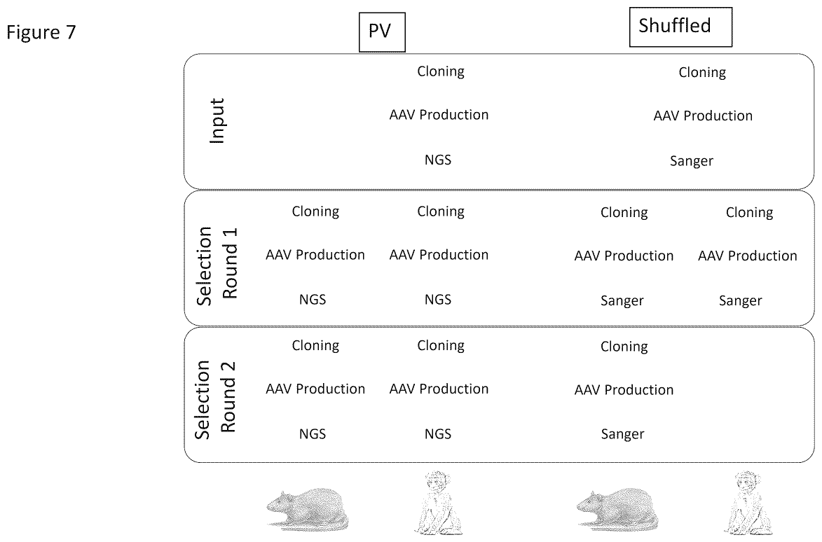

After AAV production, quality control and NGS analysis, the libraries were injected into mice or non-human primates (NHPs) for the first round of selection. One week after injection, the eyes were collected and proceeded to cell separation and DNA/RNA isolation, as previously described: retinal ON bipolar cells were identified and isolated via fluorescence-activated cell sorting (FACS) based on the expression of the intracellular marker Purkinje cell protein-2 (PCP2), a member of the GoLoco domain-containing family (Murenu E et al. Mol Ther Methods Clin Dev. 2021; 20:587-600 & Xu et al. J Neurosci. 2008; 28:8873-8884). Three cell types were isolated from each eye: PCP2 high expression, low expression and negative (no expression). From each DNA sample, a PCR was performed, the cap PCR product was digested with BsiWI and SpeI, or with PacI and AscI for the shuffled capsids, and cloned into new ITR containing plasmids for new plasmid and AAV library productions, as previously described. Only the AAV libraries from the PCP2 high population were used for the second round of selection (FIG. 7), which was performed similar to the first round. In total, 2 rounds of selection were performed for the peptide display libraries. For the shuffled libraries, 2 rounds were performed in mice and one round in NHP.

All peptide display libraries were analyzed by NGS, whereas the shuffled library was analyzed by Sanger sequencing. For the peptide display libraries, the NGS was performed on the AAV libraries as follows: round 1 PCP2 high (MoR1h) and low (MoR1l) for mice and PCP2 high (NHPR1h), low (NHPR1l) and negative (NHPR1n) for NHPs, round 2 PCP2 high for mice (MoR2h) and NHPs (NHPR2h).

From the NGS analysis it was evident that the distribution of proportions for each variant was shifted already massively after just one round of selection. Some variants exhibited high proportions in the library, whereas a lot of them showed lower than the input, which is to be expected, since some variants dominate the library after selection. The amino-acid distribution also showed a clear selection of amino acids compared to the input library after just one round of selection, whereas after the second round there were no major differences (FIG. 4). This is reasonable considering the selection pressure applied after just one round of selection. The selection was based on a limited amount of cells and DNA from a specific cell subtype. According to some rough calculations, the AAV library was generated from about 10000 cells with about 1 viral genome per genome copy (Murenu E et al., supra).

Overall, 80 AAV vectors with different peptide insertion sequences and 3 shuffled capsid AAV vectors were selected for the further screening as described in the following examples. The selected sequences were cloned into pRep2Cap plasmids that lack ITRs. Each pRep2Cap plasmid was combined with a plasmid containing an ITR-flanked, eGFP-carrying, barcode-tagged transgene for the individual manufacturing of barcoded AAV vectors, which were then mixed into a barcoded AAV vector pool as described in detail in Example 5.

### Example 5: Manufacturing of Barcoded rAAV Vector Pool

The barcoded AAV pool manufactured as described in the present example not only comprised the abovementioned 83 vectors, but further comprised the following seven benchmark vectors:
1) Wild-type AAV8 (internal reference Ku101);
2) Wild-type AAV2 (internal reference Ku98);
3) Wild-type AAV5 (internal reference Ku100);
4) Wild-type AAV4 (internal reference Ku99);
5) An AAV2 vector with tyrosine to phenylalanine mutations at positions 444, 500 and 730 (VP1 numbering), see Petrs-Silva et al. supra and Petrs-Silva et al., Molecular Therapy. 2011; 19 (2): 293-301 (internal reference Ku108; also known as triple tyrosine-mutated vector);
6) An AAV8 vector with the altered sequence of PERTAMSLP ((SEQ ID NO: 117), compared to QQQNTAPQI (SEQ ID NO:118)) in the amino acid region 585-594, see Cronin et al., EMBO Mol Med. 2014; 6:1175-1190 (internal reference Ku106; also known as AAV8.B2); and
7) An AAV2 vector with the insertion LALGETTRP (SEQ ID NO:119) at position 588 (VP1 numbering), see Dalkara et al., Science Translational Medicine. 2013; Vol. 5, Issue 189 (internal reference Ku105; also known as AAV2.7m8).

The barcoded rAAV library was manufactured in suspension cultures at the 30 mL scale using the AAV-MAX system from Thermo Fisher Scientific. In detail, VPC 2.0 cells were grown in Gibco™ Viral Production Medium supplemented with 4 mM GlutaMAX™ at 37° C., 80% relative humidity and 8% CO₂. Cells were maintained in the log phase and diluted to 3×10⁶ viable cells/mL for transfection using 45 μg total plasmid DNA per culture with a ratio of pHelper to pRep2Cap to pTransgene of 1:3:1. The pTransgene was an assortment of plasmids encoding an ITR-flanked eGFP under the control of the CMV promoter. Each GFP in the collection had a unique 38nt-long barcode sequence at the 3′UTR. One barcoded eGFP was assigned to one specific rAAV peptide variant.

The rAAVs were harvested three days post transfection by pelleting the cells via centrifugation, then resuspending them in 10 mL lysis buffer (5 mM Tris-Cl, pH 8.5, 150 mM NaCl, 5 mM MgCl₂ and 0.001% Pluronic F68). These were stored frozen until further processing. rAAV vectors in the growth medium were precipitated from solution by adding PEG-8000 and NaCl to a final concentration of 10% and 625 mM, respectively, then incubating overnight at 4° C. The PEG-precipitated rAAV vectors were collected by centrifuging at 3800×g for 30 minutes, pooled with the frozen cell pellet material, then topped up to 20 mL with lysis buffer. Lysis and nucleic acid digest were done with 0.1% Triton X-100 and 25 units/mL Benzonase® endonuclease at 37° C. for 2 hours. The lysates were clarified by centrifugation at 14000×g for 10 minutes. To separate the rAVV vectors carrying the GFP transgene from those that were empty, 19 mL of clarified lysate were added to a Beckman Coulter Quick Seal® centrifuge tube and overlaid with OptiPrep (15%, 25%, 40% and 60%.; Progen) as previously described (Zolotukhin, S. et al. Gene Ther. 1999; 6:973-985). The rAAV vectors were formulated in PBS containing 0.001% Pluronic® F68 using Amicon® Ultra 15 mL centrifugal concentrators then passed through a 0.2 μm syringe-filter.

### Example 6: RAAV Titer Determination by PCR

The goal was to generate a library with equimolar amounts of each rAAV when starting from the barcoded rAAV vector library from Example 5. rAAV vector genomes were liberated from the capsids by adding SDS to a final concentration of 0.56% then heating at 65° C. for 15 minutes. Genome copy numbers were quantified using qPCR and specific ITR primers by interpolating from a plasmid standard curve.

The assignment of each individual barcoded sequence to its AAV variant or AAV benchmark vector, respectively, was confirmed by PCR for each individual produced AAV and by sequencing of the packaged DNA. Barcodes were assigned using specific primers flanking the barcode sequence and capsids were assigned using primers flanking the peptide insertion site in the RepCap plasmid. The latter approach makes use of the fact that low amounts of plasmids, pRep2Cap and pHelper, being co-transfected for production, as well as sequences close to the ITRs, but not within the transgene, are found to be co-packaged into the AAV capsids as contaminants (Brimble MA. et al. Mol. Ther. 2023; 31:2826-2838).

### Example 7: Variant Distribution Determination

Next-generation sequencing (NGS) of the barcoded rAAV vector library (input library sequencing) was performed. The goal of this example was to quantify the exact distribution of each rAAV vector or barcode in the input library. This quantification was used to normalize the data received in single nucleus sequencing and bulk sequencing.

Sequencing libraries representing the relative abundance of AAV variant barcodes in the input material were generated in three PCR steps using NEBNext Q5 Hot Start HiFi PCR Master Mix (New England Biolabs, USA) and following the manufacturer's recommendations except for choosing 69° C. for the elongation step, in order to avoid barcode swapping. A dilution series of AAV variant pool was processed. The following primers were used to amplify the pooled AAV library (Table 3).

TABLE 3
Primers used to amplify the pooled AAV library.
(i5) indicates the position of an 8 bp long
Illumina i5 index for multiplexing and (i7)
indicates the position of an 8 bp long
Illumina i7 index for multiplexing.

| | | SEQ ID |
|---|---|---|
| Primer | Sequence (5′ -> 3′) | NO: |
| 3941 | CGAGACGCTCCTTCCTCTCA | 106 |
| 3885 | GTAGATCTCTCGAGCAGCATCTCG | 107 |
| 3888 | TCTTTCCCTACACGACGCTCTTCCGAT | 108 |
| | CTNNNNNNNNNNNNGTAGATCTCTCGA | |
| | GCAGCATCTCG | |
| 3890 | GTGACTGGAGTTCAGACGTGTGCTCTT | 109 |
| | CCGATCTGCCACCTCCCACCTAGGCTA | |
| 3305 | AATGATACGGCGACCACCGAGATCTAC | 110 |
| | AC(15)ACACTCTTTCCCTACACGACG | |
| | C | |
| 3117 | CAAGCAGAAGACGGCATACGAGAT(i7) | 111 |
| | GTGACTGGAGTTCAGACG | |

In the first PCR, the viral DNA copies were released from the nucleocapsid by heat denaturing, and the barcode cassette was amplified using primers 3941 (SEQ ID NO:106) and 3885 (SEQ ID NO:107). In the second PCR, read adapters were added with the primers 3890 (SEQ ID NO:109) and 3888 (SEQ ID NO:108). In the third round of PCR, Illumina sample indices and sequencing adapters were added, using 3117 (SEQ ID NO:111) for i7/P7 and 3305 (SEQ ID NO:110) for i5/P5. All PCRs were stopped in the exponential phase.

NGS libraries were sequenced in paired-end mode, capturing the viral barcodes in read 2. Read 2 sequences were processed with a custom-made bash script. In short, sequences were trimmed using the flanking constant regions of the barcode cassette and mapped to the barcode reference library as well as counted with bowtie2. Read counts were normalized to library size using the CPM (counts per million) method to yield relative barcode abundances, making the libraries comparable.

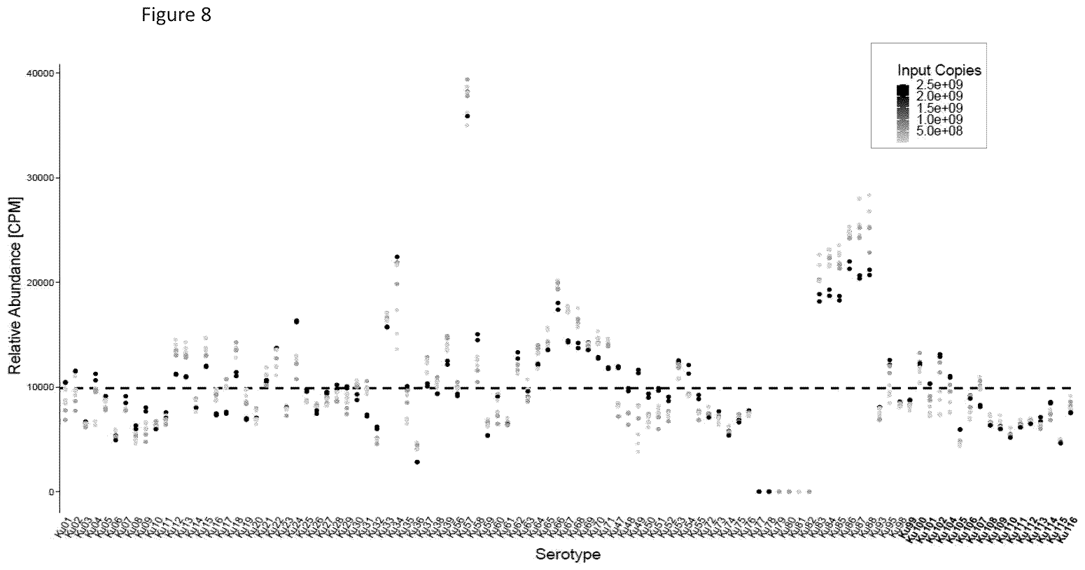

The sequencing results revealed an even distribution (dashed black line) of most AAVs/barcodes with few outliers (FIG. 8).

This input barcoded rAAV vector library was then used in in vivo experiments.

### Example 8: In Vivo Non-Human Primate Subretinal Study

The retinal cell types and their fundamental neuroanatomical arrangement is conserved across vertebrate species. However, there are substantial differences in gross anatomy between primates (including humans) and all other vertebrates, which impact the access of intra-ocular delivered virus to the target retinal cell types. For this reason, any pre-clinical assessment of viral transduction efficiency must realistically be undertaken in a species that shares this fundamental anatomical feature of the human retina. To this end, the AAV variants were validated and analyzed in vivo in non-human primates (NHP). Two NHPs (1 male/1 female) were injected subretinally at a dose of 2×10¹¹ vg/eye. Both eyes of each animal were dosed with 2 blebs/eye. After an in-life phase of 4 weeks and necropsies, the neuroretina was separated from the RPE, and punches taken from the bleb areas, maculae, and periphery (Table 4 below) were snap-frozen.

In order to analyze the viral spread across the optical pathway, extra-orbital samples (Off-Target tissue) were acquired from: (i) Optic nerve retrobulbar, (ii) Optic chiasma, (iii) Lateral Geniculate Nucleus; and (iv) Visual cortex.

For the ON-target tissues, nuclei were isolated and single nucleus RNAseq was performed (Example 9). Viral spread was quantified, after DNA isolation, in both ON- and OFF-target tissues by qPCR (vg/cell) (see Example 10). Finally, the viral distribution on DNA and RNA level on ON-target tissues was performed in bulk by NGS (Examples 10 and 11).

TABLE 4
Sample overview
In vivo- NHP Neuroretinal samples for snRNAseq

| Gender | eye left/right | retinal area |
|---|---|---|
| male | left | superior bleb |
| male | right | superior bleb |
| female | left | superior bleb |
| female | right | superior bleb |
| female | left | macula |
| female | right | macula |
| female | left | periphery nasal |

### Example 9: Preparation of Nuclei from Retina and Transcriptomic (cDNA) Library Construction and Sequencing

The goal of this experiment was to prepare (a) endogenous RNA for gene expression analysis, and (b) to rescue viral RNA (barcode) for viral gene expression analysis.

Nuclei were released from cryo-conserved neuroretinal tissue by incubation in a hypotonic isolation buffer, followed by mechanical processing and the addition of a mild detergent. The released nuclei were isolated by discontinuous density gradient centrifugation using OptiPrep™ Iodixanol. Isolated nuclei were resuspended, filtered, and counted before loading.

For the generation of single nucleus barcoded cDNA transcripts and gene expression (GEX) NGS libraries, Chromium NextGEM Single Cell 3′ Reagent Kits v3.1 (10×Genomics, USA) were used following the manufacturer's instructions.

Viral transcript-enriched sequencing libraries were generated in a semi-nested two-step PCR with the NEBNext Q5 Hot Start HiFi PCR Master Mix (New England Biolabs, USA) following the manufacturer's recommendations except for choosing 69° C. for the elongation step in order to avoid barcode swapping. The following primers were used (Table 5):

TABLE 5
Primers used to amplify the Viral transcript-
enriched sequencing libraries. (i5) indicates
the position of an 8 bp long Illumina i5
index for multiplexing

| Primer | Sequence (5′ -> 3′) | SEQ ID |
|---|---|---|
| | | NO: |
| 3901 | TCTTTCCCTACACGACGCTCTTCCGAT | 112 |
| | CT | |
| 3941 | CGAGACGCTCCTTCCTCTCA | 106 |
| 3885 | GTAGATCTCTCGAGCAGCATCTCG | 107 |
| 3854 | CAAGCAGAAGACGGCATACGAGATAGA | 113 |
| | GTGGAGTGACTGGAGTTCAGACGTGTG | |
| | CTCTTCCGATCTGCCACCTCCCACCTA | |
| | GGCTA | |
| 3305 | AATGATACGGCGACCACCGAGATCTAC | 110 |
| | AC(i5)ACACTCTTTCCCTACACGACGC | |

In the first PCR step, viral transcripts in the single nucleus barcoded cDNA libraries were enriched using an outer primer specific to the barcode expression cassette (3941; SEQ ID NO:106) and a reverse primer binding the 10×Genomics PCR handle (3901; SEQ ID NO:112). In the second PCR step, sequencing adapters and Illumina sample indices were added with an inner primer specific to the 5′ constant region of the barcode cassette (3854, SEQ ID NO:113) and a sample index primer binding to the 10× Genomics PCR handle (3305, SEQ ID NO:110) enabling sequencing of the 10× cell barcode and UMI (unique molecular identifier) in read 1.

Sequencing libraries were pooled for Illumina sequencing. Cell barcode and UMI were sequenced in read 1, transcript and barcode sequences were sequenced in read 2. Targeted sequencing depth range was 30 k to 40 k GEX reads per cell and 3 k to 6 k viral barcode reads per cell.

### Example 10: GDNA/RNA Extraction and qPCR

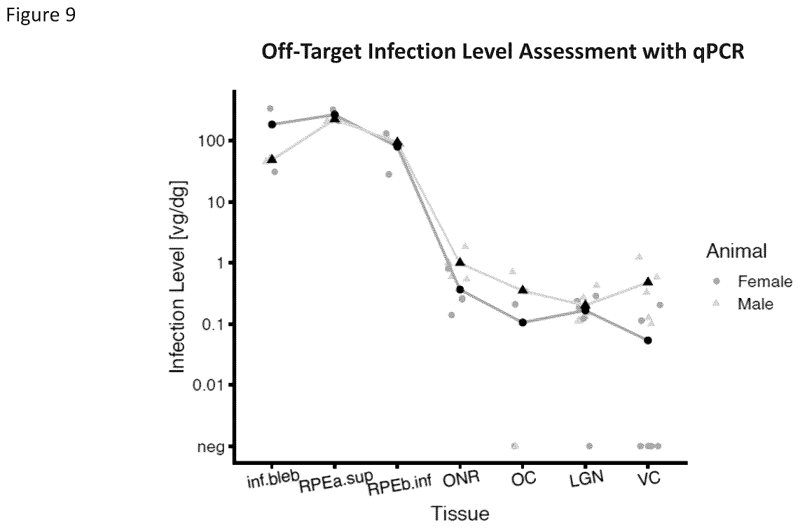

The goal was to analyze the overall viral spread outside the retina and to quantify the total viral genomes over diploid genomes (VG/DG) by qPCR in OFF-target tissues of the optical pathway, i.e., in the optical nerves, in the optical chiasma, in the lateral geniculate nucleus (LGN), and in the optical cortex. Additional ON-target samples (inferior bleb and retinal pigment epithelium, RPE) were included. The analysis of the VG/DG (cell) showed a clear drop in the infection level in OFF-target tissues (optical nerves (ONR), optical chiasma (OC), lateral geniculate nucleus (LGN) and virtual cortex (VC) (FIG. 9).

Samples were homogenized on ice using a rotor-stator mixer. Both DNA and RNA were extracted with the AllPrep DNA/RNA Mini kit (Qiagen). PCRs for viral barcodes (viral genomes, VG) were performed as described in Example 6, except for using primer 3852 instead of primer 3941 during the first amplification step (Table 6). This change was made to generate a shorter 144 bp product better suited for qPCR analyses. Primers for genomic GAPDH amplification to determine cell numbers (diploid genomes, DG) were 4035 and 4036. All PCRs were performed in triplicates, including a plasmid standard curve of 1 copy to 10 Mio copies for absolute quantification.

TABLE 6
Primers used for the amplification
of the viral codes

| Primer | Sequence (5′ -> 3′) | SEQ ID NO: |
|---|---|---|
| 3852 | GCCACCTCCCACCTAGGCTA | 114 |
| 3885 | GTAGATCTCTCGAGCAGCATCTCG | 107 |
| 4035 | AAAGCTGATGTGGGAGGAGC | 115 |
| 4036 | TTCCCGTTCTCAGCCTTCAC | 116 |

Data processing was as follows: 1) input adjustment according to minor differences in GAPDH Ct values, compared to average; 2) determination of AAV barcode copy numbers (VG) via the standard curve; and 3) calculation of VG per cell number DG (15.4 ng DNA input corresponds to 2.5K diploid macaque genomes).

### Example 11: Bulk Tissue NGS Analysis and AAV Distribution in Retinal Pigment Epithelium (RPE)

RPE tissue samples were collected in the bleb region after subretinal AAV injection into 4 NHP eyes, 4 week NHP in-life phase, and necropsy of the eyes. Viral DNA (vDNA) from the previous qPCRs (Example 10) was directly processed for bulk sequencing (10 PCR cycles with Illumina index primers) since the PCR shows no bias towards particular barcodes and can be driven into saturation (tested by sequencing of a ‘barcode ladder’ amplified at 5-40 cycles). For bulk sequencing of viral RNA (barcodes) in each RPE sample, 100 ng were reverse-transcribed, and 40 ng of re-quantified cDNA were used to amplify RNA-derived barcodes for bulk sequencing. All RPE samples were sequenced individually for both vDNA and vRNA.

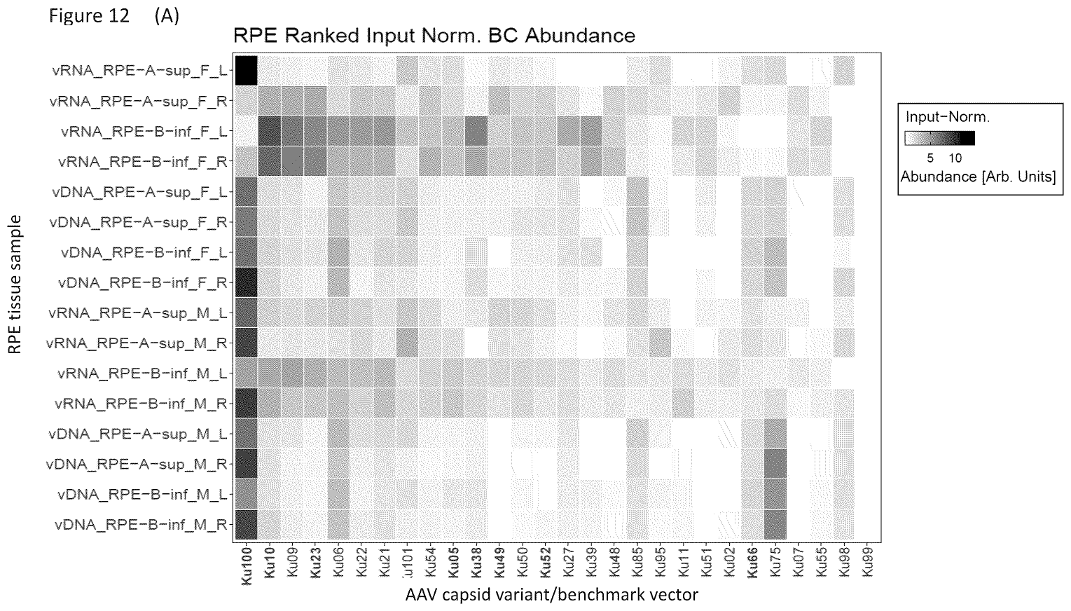

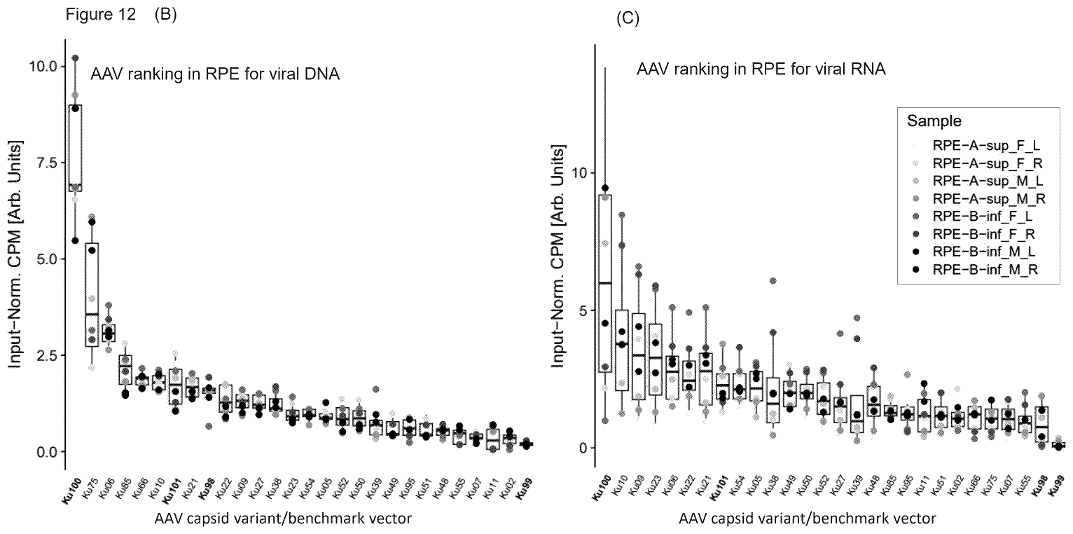

Sequencing libraries were pooled for Illumina sequencing. Reads were transformed to relative abundance (counts per million, CPM), normalized to the corresponding abundances in the input material (Example 7), and ranked based on their median vRNA (cDNA) abundance in RPE vDNA samples (FIGS. 12A and C) or ranked based on their median vDNA abundance (FIG. 12B).

As shown in FIG. 12, the AAV ranking order differs between vDNA and vRNA samples for most AAV capsid variants and benchmark vectors. For example, AAV capsid variants Ku10, Ku09, Ku23, Ku21, Ku06 and Ku22 show higher abundance on vRNA level, i.e., improved transcriptional efficiencies in the RPE compared to benchmark vectors Ku101, Ku 98 and Ku 99 (see FIG. 12 (C)). In contrast, Ku10, Ku09, Ku23, Ku21, Ku06 and Ku22 show lower or similar abundance on vDNA level, i.e., a lower or similar transduction level in the RPE compared to benchmark vectors Ku101, Ku 98 and Ku 99 (see FIG. 12 (B)). Ku100 (AAV5) shows slightly increased transcription efficiency over Ku10 (vRNA levels, FIG. 12(C)). However, this effect is achieved at >3-fold higher vDNA level (FIG. 12 (B)).

Accordingly, selected AAV capsid variants (e.g. Ku10, Ku 09, Ku23, Ku21, Ku06, Ku22) show a favorable vRNA/vDNA abundance ratio, indicating efficient viral transcription and functionality of these variants at the same low vDNA abundance. As mentioned above, the benchmark vector Ku100 shows comparable viral transcription to AAV capsid variant Ku10 (see FIG. 12 (C)), however, it shows ˜3× times higher vDNA abundance (see FIG. 12 (B)), i.e., an unfavorable vRNA/vDNA abundance ratio. These results indicate the above-mentioned AAV capsid variants are more efficiently transcribed from their respective viral DNA template, which is one prerequisite for therapeutic transgene expression. In addition, the above indicated AAV capsid variants with a favorable vRNA/vDNA abundance ratio, i.e., the ones requiring less vDNA template, show that they have improved AAV capsid functionality in the RPE, thereby delivering successfully the payload to the RPE nuclei for transcription. This demonstrates the usefulness of the AAV capsid variants for efficient therapeutic transgene expression in the RPE for gene therapy treatment of diseases such as dry AMD.

### Example 12: Gene Expression Analysis Pipeline

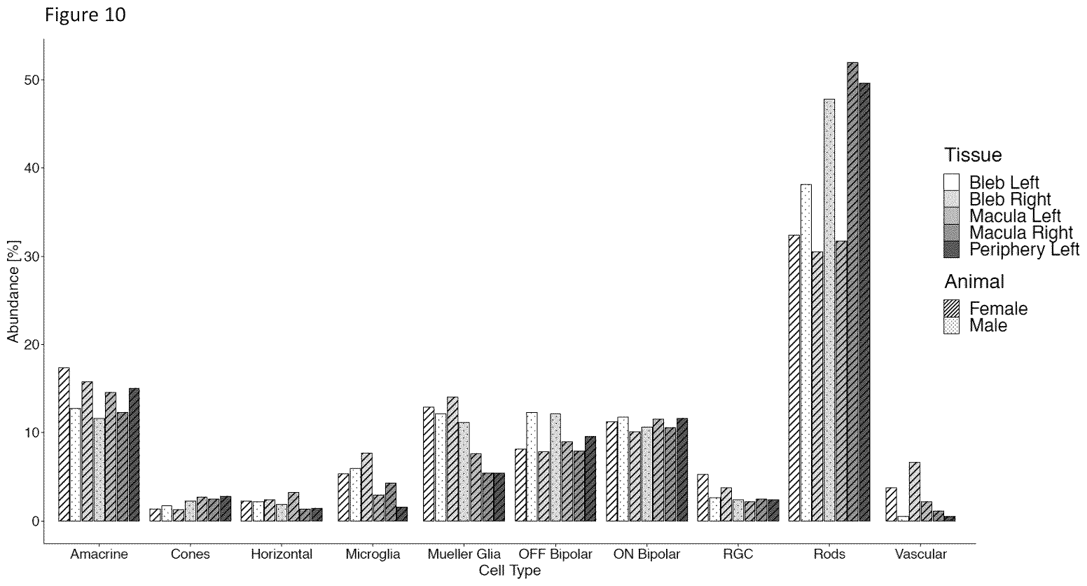

The goal here was to analyze and to quantify distribution of each of the rAAV vectors in the pool in the cellular layers of the retina. First, endogenous gene expression of each nucleus was analyzed to identify cell type specific markers and assign cell populations in the retina. All tested retinal samples showed a similar distribution of retinal cell populations (FIG. 10).

The analysis of barcode-positive cells showed how many cells of a given population contain a specific barcode. In general, a high percentage of >50% for each cell type was infected by most of the rAAV vectors (barcodes), however this method does not count the number of viral transcripts (barcodes).

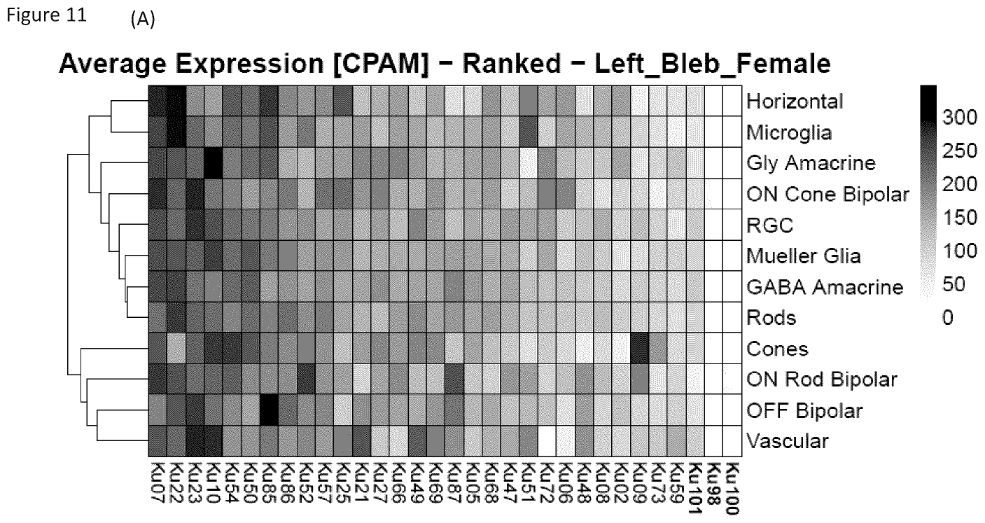

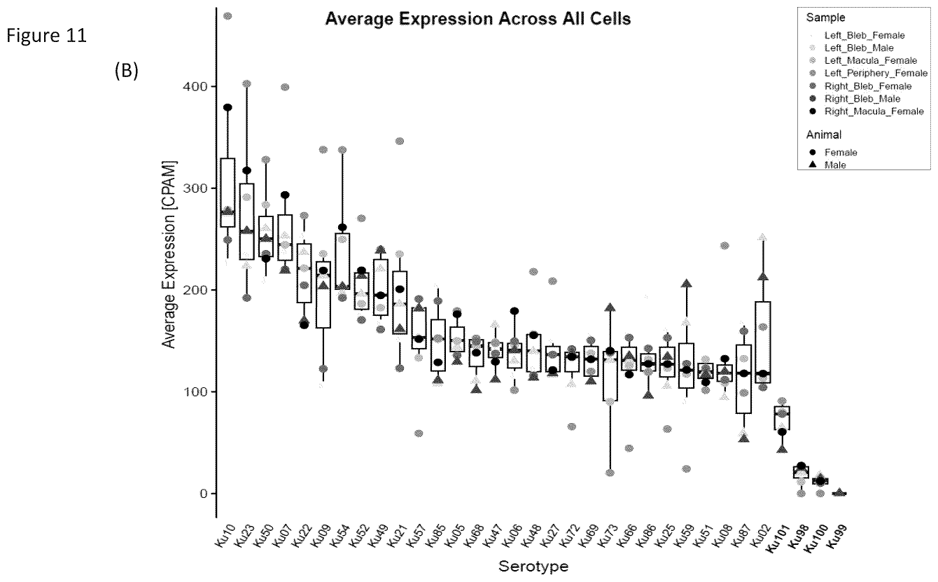

The transduction efficiency was determined by analyzing the average expression of viral transcripts (barcodes) in a cell population, such as left bleb samples (FIG. 11A, wherein FIG. 11A only shows a single sample of the total seven samples) and across all cells (FIG. 11B). AAV expression data were divided in barcode frequencies in the original AAV input libraries to compensate for deviations from a uniform distribution in the injected material. The resulting data was divided by the gene expression library size to compensate for differences in RNA recovery and general transcriptional activity. The resulting values were scaled by 106 and log-normalized to yield counts per abundance-adjusted million (CPAM). This analysis showed a clear ranking and improved transduction efficiencies of selected rAAV vector variants compared to the seven benchmarks included in the pool, as discussed further below.

The CellRanger v7.0 count pipeline (10×Genomics, USA) was used for cell barcode tagging, mapping of the transcript reads, filtering, and identification of cell-associated barcodes. Further, the feature barcode analysis function was used to extract, tag, and count viral transcript reads from the enrichment libraries. For GEX reads, a custom reference for Macaca fascicularis was built from the primary genome assembly (GCF_012559485.2_MFA1912RKSv2) and the corresponding NCBI annotation (release 102) available through NCBI RefSeq. For viral barcode reads, a custom feature barcode table following 10×Genomics' recommendations was built. CellRanger was also used for a general quality check of the data. Samples were included in the analysis only, if they were found to have enough cell-associated barcodes with sufficient complexity per cell and acceptable background levels.

The count matrices from CellRanger's output were preprocessed and analyzed further using custom-made R scripts as explained in the following. The first script removed chimeric reads and estimated as well as removed background transcripts to avoid cross-contamination between nuclei. The output of the first script was two filtered matrices: one for endogenous transcripts (GEX) and one for viral transcripts (AAV). The second R script used the GEX matrix to filter for good quality nuclei and to call their cell type. In short, doublets were annotated, the matrix was then filtered for singletons with at least 800 identified genes. In addition, nuclei with a notable mitochondrial transcript content were removed, because high mitochondrial content in single-nucleus RNA sequencing data indicates considerable cytoplasmic contamination. The resulting matrix should only contain data from high quality single nuclei with low ambient RNA contribution. The filtering steps were followed by a normalization step, using the single-cell transform (SCT) method, dimension reduction by a principal component analysis (PCA), and SNN clustering of the cells. For visualization purposes a UMAP embedding of the gene expression data was computed. The cell clusters were integrated with a publicly available reference dataset (Peng et al. Cell. 2019; 176:1222-1237) to identify the cell type represented by each cluster. In order to validate the called cell type, the expression of a list of curated cell type marker genes was verified.

After analysis of the GEX data, the viral transcript count matrix (AAV) was added to the filtered dataset and analyzed with a third custom-made R script. The AAV data was filtered for the cells passing the GEX quality control process and annotated with the cell type identified in the GEX analysis.

The rate of AAV barcode-positive cells was determined in the same custom R script. Since a normalization strategy analogous to the one described below for average expression is not adequate for detection rate data, interpretation of the positive cell rate will still be influenced by confounding factors such as sequencing depth, sample quality and over- or underrepresentation of a given capsid in the injected material.

The viral barcode expression data was normalized in several steps to account for fluctuations from biological sources and technical artifacts between samples, runs and variants. The normalization procedure is explained in more detail in Example 13. It allows for semi-quantitative comparisons between cell populations and capsid variants based on average expression of viral transcript in a given cell population. Average expression was computed for cell populations of interest by taking the mean and log-transforming the result. Differences between cell populations in average expression of a variant's barcode indicate cell type tropism of that AAV variant. Since most confounding factors impacting between-sample comparisons are taken care of with the normalization strategies used, side-by-side comparisons of samples are generally possible. Some biological differences between tissue samples, such as cell composition, health and recovered cell number are not well controlled and should be kept in mind.

The performance of variants in specific cell types in the seven samples was compared by ranking them by the median average expression over all samples. FIG. 11A shows a single example (the left bleb of a female) and is illustrating the difference in expression in the cell types as indicated on the right, and compared with the wild-type AAV benchmarks (in bold) AAV8 (Ku101), AAV2 (Ku98) and AAV5 (Ku100). The performance of variants across the seven samples was compared by ranking them by the median average expression. Therefore, if all seven samples are taken into account, the average expression (median) of each of the different rAAV vectors with inserted peptides is as indicated in Table 7 (with the peptide-sequences of the internal IDs (Ku-numbers) given in Table 8 further below):

TABLE 7
Peptide variants selected via expression in specific cell types
(ranging from rank 1 being the highest expression to rank 10
being the lowest expression amongst the top 10 variants).

| | | | ON Rod | ON Cone | OFF | |
|---|---|---|---|---|---|---|
| Rank | Rods | Cones | Bipolar | Bipolar | Bipolar | RGC |
| 1 | Ku10 | Ku10 | Ku10 | Ku23 | Ku10 | Ku10 |
| 2 | Ku23 | Ku54 | Ku07 | Ku10 | Ku23 | Ku23 |
| 3 | Ku50 | Ku50 | Ku23 | Ku50 | Ku50 | Ku07 |
| 4 | Ku07 | Ku09 | Ku22 | Ku07 | Ku07 | Ku50 |
| 5 | Ku22 | Ku07 | Ku54 | Ku54 | Ku09 | Ku09 |
| 6 | Ku09 | Ku23 | Ku50 | Ku22 | Ku54 | Ku22 |
| 7 | Ku54 | Ku21 | Ku09 | Ku09 | Ku22 | Ku54 |
| 8 | Ku49 | Ku52 | Ku52 | Ku52 | Ku49 | Ku49 |
| 9 | Ku52 | Ku49 | Ku49 | Ku21 | Ku21 | Ku52 |
| 10 | Ku21 | Ku22 | Ku48 | Ku49 | Ku52 | Ku21 |

FIG. 11B shows the 29 best peptide variants (SEQ ID NO: 01-29) (not in bold), which performed better than the wild-type AAV benchmarks (in bold) AAV8 (Ku101), AAV2 (Ku98), AAV5 (Ku100) and AAV4 (Ku99).

All 29 best peptide variants shown in FIG. 11B not only performed better than the abovementioned wild-type AAV benchmarks, but also performed better than the abovementioned triple tyrosine-mutated vector, namely wt AAV2 with tyrosine to phenylalanine mutations at positions 444, 500 and 730 (VP1 numbering) (internal reference Ku108; not shown in FIG. 11B, the performance of this benchmark vector was comparable to wild-type AAV5 (Ku100)).

The best 13 peptide variants shown in FIG. 11B (with the sequences of SEQ ID NOs: 01 to 13) performed better than the abovementioned vector disclosed in Dalkara et al., supra, which has an AAV2 backbone and the following insertion at position 588 (VP1 numbering): LALGETTRP (SEQ ID NO: 119) (internal reference Ku105; not shown in FIG. 11B).

The best seven peptide variants shown in FIG. 11B (with the sequences of SEQ ID NOs: 01 to 07) performed better than the abovementioned vector disclosed in Cronin et al., supra, which has an AAV8 backbone and an altered sequence of the AAV8 capsid in amino acid region 585-594, namely PERTAMSLP (SEQ ID NO: 117, internal reference Ku105), compared to QQQNTAPQI (SEQ ID NO: 118) in AAV8 wild-type (not shown in FIG. 11B).

The obtained results are robust between samples and support the ranking and between-sample comparisons with the given data. The amino acid sequences of the top 29 expressed peptide variants are provided in Table 8 below.

TABLE 8
Peptide variants selected due to higher expression across all retina cells

| | R or S | 7-mer Peptide | SEQ ID NO: | |
|---|---|---|---|---|
| Internal | preceding | insertion | of the | AAV |
| ID | the insertion | sequence | 7-mer | serotype |
| Ku10 | R | RFQSNPM | SEQ ID NO: 01 | AAV2 |
| Ku23 | R | SNVVPVG | SEQ ID NO: 02 | AAV8 |
| Ku50 | R | MMHHDSH | SEQ ID NO: 03 | AAV2 |
| Ku07 | R | KVTAPHS | SEQ ID NO: 04 | AAV2 |
| Ku22 | S | AHAKGMD | SEQ ID NO: 05 | AAV8 |
| Ku09 | S | LHKNYGV | SEQ ID NO: 06 | AAV2 |
| Ku54 | R | VIGIKGA | SEQ ID NO: 07 | AAV2 |
| Ku52 | R | PHVMVPV | SEQ ID NO: 08 | AAV2 |
| Ku49 | R | MELGSFT | SEQ ID NO: 09 | AAV2 |
| Ku21 | R | MVPGKGD | SEQ ID NO: 10 | AAV8 |
| Ku57 | R | NTIRREN | SEQ ID NO: 11 | AAV2 |
| Ku85 | S | NGVKDRH | SEQ ID NO: 12 | AAV8 |
| Ku05 | R | NVDRPRA | SEQ ID NO: 13 | AAV2 |
| Ku68 | S | NDSRARM | SEQ ID NO: 14 | AAV8 |
| Ku47 | R | KAVTTDI | SEQ ID NO: 15 | AAV2 |
| Ku06 | R | IFPSTTK | SEQ ID NO: 16 | AAV2 |
| Ku48 | R | EHKAIMM | SEQ ID NO: 17 | AAV2 |
| Ku27 | S | LKDAVVK | SEQ ID NO: 18 | AAV8 |
| Ku72 | R | NNVMKHS | SEQ ID NO: 19 | AAV2 |
| Ku69 | S | NAVRMAP | SEQ ID NO: 20 | AAV8 |
| Ku73 | R | NATKVSW | SEQ ID NO: 21 | AAV2 |
| Ku66 | S | NSTRATM | SEQ ID NO: 22 | AAV8 |
| Ku86 | R | MNDTSRS | SEQ ID NO: 23 | AAV8 |
| Ku25 | R | NVVRPVA | SEQ ID NO: 24 | AAV2 |
| Ku59 | R | NQVRHFS | SEQ ID NO: 25 | AAV2 |
| Ku51 | R | KDSIERF | SEQ ID NO: 26 | AAV2 |
| Ku08 | S | NFRHHGL | SEQ ID NO: 27 | AAV2 |
| Ku87 | S | NEMNRSR | SEQ ID NO: 28 | AAV8 |
| Ku02 | R | NHDRGTN | SEQ ID NO: 29 | AAV2 |

### Example 13: Normalization Strategies

The goal was to account for differences in the input amounts of the individual AAVs and for differences in the genomic transcription levels of the analyzed retinal cell populations.

Normalization was applied to allow for the comparison of individual AAVs and their transduction efficiencies on the RNA level in the identified cell populations.

The normalization procedure was done using a custom R script and was comprised of three main steps. In the first step, AAV counts were normalized to the GEX library size. This procedure compensates for RNA recovery differences between single nuclei, e.g., due to fluctuations in RNA quality and nucleus integrity, as well as differences between cell types in transcript content. These depend on factors such as transcriptional activity and nuclear export rate, which are actively regulated and can differ from cell type to cell type. In the samples described in Example 12, the cone photoreceptor cell and retinal ganglion cell clusters exhibit globally higher GEX and AAV transcript counts than other clusters, indicating a greater amount of RNA recovered. This observation is most likely to be explained by a generally higher amount of RNA present in their nuclei. This inflates the rate of AAV barcode-positive cells observed in the data (as shown in Example 12, FIG. 11B), which in turn leads to an overestimation of the overall infection rate.

When comparing serotypes within a given cell population, this factor has no influence on the results except for a potential boundary effect for very high detection rates close to or reaching 100%. When comparing different cell populations, however, it is important to compensate for the higher detection rate, otherwise the results are skewed in favor of the above-mentioned cell clusters. This is handled by normalization to the total number of endogenous (GEX) transcript counts, adjusting for differences in the total transcript number recovered. At the same time, different levels of infectivity are preserved, by using the GEX library size as reference instead of the total number of AAV counts. In the second normalization step, differences in the abundance of single variants in the injected tissues were compensated based on the input material sequencing data acquired earlier. In the third step, the resulting values were scaled to reduce the number of decimals needed for the analysis.

### Example 14: Ex Vivo Infection in Human Retina

Selected AAV variants (Ku7, Ku9, Ku10, Ku22, Ku23 and Ku50) and benchmarks (wtAAV2 (Ku98), wtAAV5 (Ku100), wtAAV8 (Ku101), modified wtAAV2 (Ku105) vectors were evaluated ex vivo in human retina explants. AAV vectors were manufactured individually, each AAV packaging an eGFP expression cassette under a CMV promoter. Chorioretinal explants were taken from healthy deceased donors. The explants were taken from the macular and the peripheral regions of the retina and incubated individually with AAVs for 5 days. The indicated variants or benchmark AAVs were applied individually to each explant, i.e., one AAV per explant. wtAAV2 was applied to a macular explant only, wtAAV5 and Ku09 were applied to peripheral explants, only. All other AAV variants were applied to both, a macular and to a peripheral explant.

In particular, to prepare the retina samples, after removing the optic nerve, cornea, iris, and lens—while preserving the ora serrata—the eye was flattened and chorioretina explants were isolated using a 5 mm biopsy punch, maintaining anatomical orientation.

Explants were placed ganglion side up on transwell membranes. Once all samples were collected, the explants were incubated individually with the AAV vectors and cultured at 37° C. with 5% CO₂, with media changes every 48 hours. On day 5, explants were fixed in 4% PFA, cryoprotected through graded sucrose solutions, embedded in Optimal Cutting Temperature (OCT), and frozen for further analysis.

AAV distribution in the retina is analyzed by confocal microscopy and GFP imaging.

Wild-Type Capsid Amino Acid Sequences
(SEQ ID NO: 30 to 45)
wild-type capsid amino acid sequence of AAV2
SEQ ID NO: 31
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYYLSRTNT
PSGTTTQSRLQFSQAGASDIRDQSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GNRQAATADVNTQGVLPGMVWQDRDVYLQGPIWAKIPHTDGHFHP
SPLMGGFGLKHPPPQILIKNTPVPANPSTTFSAAKFASFITQYST
GQVSVEIEWELQKENSKRWNPEIQYTSNYNKSVNVDFTVDTNGVY
SEPRPIGTRYLTRNL
wild-type capsid amino acid sequence of AAV3
SEQ ID NO: 32
MAADGYLPDWLEDNLSEGIREWWALKPGVPQPKANQQHQDNRRGL
VLPGYKYLGPGNGLDKGEPVNEADAAALEHDKAYDQQLKAGDNPY
LKYNHADAEFQERLQEDTSFGGNLGRAVFQAKKRILEPLGLVEEA
AKTAPGKKRPVDQSPQEPDSSSGVGKSGKQPARKRLNFGQTGDSE
SVPDPQPLGEPPAAPTSLGSNTMASGGGAPMADNNEGADGVGNSS
GNWHCDSQWLGDRVITTSTRTWALPTYNNHLYKQISSQSGASNDN
HYFGYSTPWGYFDFNRFHCHFSPRDWQRLINNNWGFRPKKLSFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFQFSYTFEDVPFHSSYAHSQSLDRLMNPLIDQYLYYLNRTQG
TTSGTTNQSRLLFSQAGPQSMSLQARNWLPGPCYRQQRLSKTAND
NNNSNFPWTAASKYHLNGRDSLVNPGPAMASHKDDEEKFFPMHGN
LIFGKEGTTASNAELDNVMITDEEEIRTTNPVATEQYGTVANNLQ
SSNTAPTTRTVNDQGALPGMVWQDRDVYLQGPIWAKIPHTDGHFH
PSPLMGGFGLKHPPPQIMIKNTPVPANPPTTFSPAKFASFITQYS
TGQVSVEIEWELQKENSKRWNPEIQYTSNYNKSVNVDFTVDTNGV
YSEPRPIGTRYLTRNL
wild-type capsid amino acid sequence of AAV3b
SEQ ID NO: 33
MAADGYLPDWLEDNLSEGIREWWALKPGVPQPKANQQHQDNRRGL
VLPGYKYLGPGNGLDKGEPVNEADAAALEHDKAYDQQLKAGDNPY
LKYNHADAEFQERLQEDTSFGGNLGRAVFQAKKRILEPLGLVEEA
AKTAPGKKRPVDQSPQEPDSSSGVGKSGKQPARKRLNFGQTGDSE
SVPDPQPLGEPPAAPTSLGSNTMASGGGAPMADNNEGADGVGNSS
GNWHCDSQWLGDRVITTSTRTWALPTYNNHLYKQISSQSGASNDN
HYFGYSTPWGYFDFNRFHCHFSPRDWQRLINNNWGFRPKKLSFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFQFSYTFEDVPFHSSYAHSQSLDRLMNPLIDQYLYYLNRTQG
TTSGTTNQSRLLFSQAGPQSMSLQARNWLPGPCYRQQRLSKTAND
NNNSNFPWTAASKYHLNGRDSLVNPGPAMASHKDDEEKFFPMHGN
LIFGKEGTTASNAELDNVMITDEEEIRTTNPVATEQYGTVANNLQ
SSNTAPTTRTVNDQGALPGMVWQDRDVYLQGPIWAKIPHTDGHFH
PSPLMGGFGLKHPPPQIMIKNTPVPANPPTTFSPAKFASFITQYS
TGQVSVEIEWELQKENSKRWNPEIQYTSNYNKSVNVDFTVDTNGV
YSEPRPIGTRYLTRNL
wild-type capsid amino acid sequence of AAV4
SEQ ID NO: 34
MTDGYLPDWLEDNLSEGVREWWALQPGAPKPKANQQHQDNARGLV
LPGYKYLGPGNGLDKGEPVNAADAAALEHDKAYDQQLKAGDNPYL
KYNHADAEFQQRLQGDTSFGGNLGRAVFQAKKRVLEPLGLVEQAG
ETAPGKKRPLIESPQQPDSSTGIGKKGKQPAKKKLVFEDETGAGD
GPPEGSTSGAMSDDSEMRAAAGGAAVEGGQGADGVGNASGDWHCD
STWSEGHVTTTSTRTWVLPTYNNHLYKRLGESLQSNTYNGFSTPW
GYFDFNRFHCHFSPRDWQRLINNNWGMRPKAMRVKIFNIQVKEVT
TSNGETTVANNLTSTVQIFADSSYELPYVMDAGQEGSLPPFPNDV
FMVPQYGYCGLVTGNTSQQQTDRNAFYCLEYFPSQMLRTGNNFEI
TYSFEKVPFHSMYAHSQSLDRLMNPLIDQYLWGLQSTTTGTTLNA
GTATTNFTKLRPTNFSNFKKNWLPGPSIKQQGFSKTANQNYKIPA
TGSDSLIKYETHSTLDGRWSALTPGPPMATAGPADSKFSNSQLIF
AGPEQNGNTATVPGTLIFTSEEELAATNATDTDMWGNLPGGDQSN
SNLPTVDRLTALGAVPGMVWQNRDIYYQGPIWAKIPHTDGHFHPS
PLIGGFGLKHPPPQIFIKNTPVPANPATTFSSTPVNSFITQYSTG
QVSVQIDWEIQKERSKRWNPEVQFTSNYGQQNSLLWAPDAAGKYT
EPRAIGTRYLTHHL
wild-type capsid amino acid sequence of AAV5
SEQ ID NO: 35
MSFVDHPPDWLEEVGEGLREFLGLEAGPPKPKPNQQHQDQARGLV
LPGYNYLGPGNGLDRGEPVNRADEVAREHDISYNEQLEAGDNPYL
KYNHADAEFQEKLADDTSFGGNLGKAVFQAKKRVLEPFGLVEEGA
KTAPTGKRIDDHFPKRKKARTEEDSKPSTSSDAEAGPSGSQQLQI
PAQPASSLGADTMSAGGGGPLGDNNQGADGVGNASGDWHCDSTWM
GDRVVTKSTRTWVLPSYNNHQYREIKSGSVDGSNANAYFGYSTPW
GYFDFNRFHSHWSPRDWQRLINNYWGFRPRSLRVKIFNIQVKEVT
VQDSTTTIANNLTSTVQVFTDDDYQLPYVVGNGTEGCLPAFPPQV
FTLPQYGYATLNRDNTENPTERSSFFCLEYFPSKMLRTGNNFEFT
YNFEEVPFHSSFAPSQNLFKLANPLVDQYLYRFVSTNNTGGVQFN
KNLAGRYANTYKNWFPGPMGRTQGWNLGSGVNRASVSAFATTNRM
ELEGASYQVPPQPNGMTNNLQGSNTYALENTMIFNSQPANPGTTA
TYLEGNMLITSESETQPVNRVAYNVGGQMATNNQSSTTAPATGTY
NLQEIVPGSVWMERDVYLQGPIWAKIPETGAHFHPSPAMGGFGLK
HPPPMMLIKNTPVPGNITSFSDVPVSSFITQYSTGQVTVEMEWEL
KKENSKRWNPEIQYTNNYNDPQFVDFAPDSTGEYRTTRPIGTRYL
TRPL
wild-type capsid amino acid sequence of AAV6
SEQ ID NO: 36
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPFGLVEEG
AKTAPGKKRPVEQSPQEPDSSSGIGKTGQQPAKKRLNFGQTGDSE
SVPDPQPLGEPPATPAAVGPTTMASGGGAPMADNNEGADGVGNAS
GNWHCDSTWLGDRVITTSTRTWALPTYNNHLYKQISSASTGASND
NHYFGYSTPWGYFDFNRFHCHFSPRDWQRLINNNWGFRPKRLNFK
LFNIQVKEVTTNDGVTTIANNLTSTVQVFSDSEYQLPYVLGSAHQ
GCLPPFPADVFMIPQYGYLTLNNGSQAVGRSSFYCLEYFPSQMLR
TGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTD
NNNSNFTWTGASKYNLNGRESIINPGTAMASHKDDKDKFFPMSGV
MIFGKESAGASNTALDNVMITDEEEIKATNPVATERFGTVAVNLQ
SSSTDPATGDVHVMGALPGMVWQDRDVYLQGPIWAKIPHTDGHFH
PSPLMGGFGLKHPPPQILIKNTPVPANPPAEFSATKFASFITQYS
TGQVSVEIEWELQKENSKRWNPEVQYTSNYAKSANVDFTVDNNGL
YTEPRPIGTRYLTRPL
wild-type capsid amino acid sequence of AAV7
SEQ ID NO: 37
MAADGYLPDWLEDNLSEGIREWWDLKPGAPKPKANQQKQDNGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEG
AKTAPAKKRPVEPSPQRSPDSSTGIGKKGQQPARKRLNFGQTGDS
ESVPDPQPLGEPPAAPSSVGSGTVAAGGGAPMADNNEGADGVGNA
SGNWHCDSTWLGDRVITTSTRTWALPTYNNHLYKQISSETAGSTN
DNTYFGYSTPWGYFDFNRFHCHFSPRDWQRLINNNWGFRPKKLRF
KLFNIQVKEVTTNDGVTTIANNLTSTIQVFSDSEYQLPYVLGSAH
QGCLPPFPADVFMIPQYGYLTLNNGSQSVGRSSFYCLEYFPSQML
RTGNNFEFSYSFEDVPFHSSYAHSQSLDRLMNPLIDQYLYYLART
QSNPGGTAGNRELQFYQGGPSTMAEQAKNWLPGPCFRQQRVSKTL
DQNNNSNFAWTGATKYHLNGRNSLVNPGVAMATHKDDEDRFFPSS
GVLIFGKTGATNKTTLENVLMTNEEEIRPTNPVATEEYGIVSSNL
QAANTAAQTQVVNNQGALPGMVWQNRDVYLQGPIWAKIPHTDGNF
HPSPLMGGFGLKHPPPQILIKNTPVPANPPEVFTPAKFASFITQY
STGQVSVEIEWELQKENSKRWNPEIQYTSNFEKQTGVDFAVDSQG
VYSEPRPIGTRYLTRNL
wild-type capsid amino acid sequence of AAV8
SEQ ID NO: 38
MAADGYLPDWLEDNLSEGIREWWALKPGAPKPKANQQKQDDGRGL
VLPGYKYLGPFNGLDKGEPVNAADAAALEHDKAYDQQLQAGDNPY
LRYNHADAEFQERLQEDTSFGGNLGRAVFQAKKRVLEPLGLVEEG
AKTAPGKKRPVEPSPQRSPDSSTGIGKKGQQPARKRLNFGQTGDS
ESVPDPQPLGEPPAAPSGVGPNTMAAGGGAPMADNNEGADGVGSS
SGNWHCDSTWLGDRVITTSTRTWALPTYNNHLYKQISNGTSGGAT
NDNTYFGYSTPWGYFDFNRFHCHFSPRDWQRLINNNWGFRPKRLS
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFQFTYTFEDVPFHSSYAHSQSLDRLMNPLIDQYLYYLSR
TQTTGGTANTQTLGFSQGGPNTMANQAKNWLPGPCYRQQRVSTTT
GQNNNSNFAWTAGTKYHLNGRNSLANPGIAMATHKDDEERFFPSN
GILIFGKQNAARDNADYSDVMLTSEEEIKTTNPVATEEYGIVADN
LQQQNTAPQIGTVNSQGALPGMVWQNRDVYLQGPIWAKIPHTDGN
FHPSPLMGGFGLKHPPPQILIKNTPVPADPPTTFNQSKLNSFITQ
YSTGQVSVEIEWELQKENSKRWNPEIQYTSNYYKSTSVDFAVNTE
GVYSEPRPIGTRYLTRNL
wild-type capsid amino acid sequence of AAV9
SEQ ID NO: 39
MAADGYLPDWLEDNLSEGIREWWALKPGAPQPKANQQHQDNARGL
VLPGYKYLGPGNGLDKGEPVNAADAAALEHDKAYDQQLKAGDNPY
LKYNHADAEFQERLKEDTSFGGNLGRAVFQAKKRLLEPLGLVEEA
AKTAPGKKRPVEQSPQEPDSSAGIGKSGAQPAKKRLNFGQTGDTE
SVPDPQPIGEPPAAPSGVGSLTMASGGGAPVADNNEGADGVGSSS
GNWHCDSQWLGDRVITTSTRTWALPTYNNHLYKQISNSTSGGSSN
DNAYFGYSTPWGYFDFNRFHCHFSPRDWQRLINNNWGFRPKRLNF
KLFNIQVKEVTDNNGVKTIANNLTSTVQVFTDSDYQLPYVLGSAH
EGCLPPFPADVFMIPQYGYLTLNDGSQAVGRSSFYCLEYFPSQML
RTGNNFQFSYEFENVPFHSSYAHSQSLDRLMNPLIDQYLYYLSKT
INGSGQNQQTLKFSVAGPSNMAVQGRNYIPGPSYRQQRVSTTVTQ
NNNSEFAWPGASSWALNGRNSLMNPGPAMASHKEGEDRFFPLSGS
LIFGKQGTGRDNVDADKVMITNEEEIKTTNPVATESYGQVATNHQ
SAQAQAQTGWVQNQGILPGMVWQDRDVYLQGPIWAKIPHTDGNFH
PSPLMGGFGMKHPPPQILIKNTPVPADPPTAFNKDKLNSFITQYS
TGQVSVEIEWELQKENSKRWNPEIQYTSNYYKSNNVEFAVNTEGV
YSEPRPIGTRYLTRNL
wild-type capsid amino acid sequence of AAV10
SEQ ID NO: 40
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEA
AKTAPGKKRPVEPSPQRSPDSSTGIGKKGQQPAKKRLNFGQTGES
ESVPDPQPIGEPPAGPSGLGSGTMAAGGGAPMADNNEGADGVGSS
SGNWHCDSTWLGDRVITTSTRTWALPTYNNHLYKQISNGTSGGST
NDNTYFGYSTPWGYFDFNRFHCHFSPRDWQRLINNNWGFRPKRLS
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFEFSYTFEDVPFHSSYAHSQSLDRLMNPLIDQYLYYLSR
TQSTGGTQGTQQLLFSQAGPANMSAQAKNWLPGPCYRQQRVSTTL
SQNNNSNFAWTGATKYHLNGRDSLVNPGVAMATHKDDEERFFPSS
GVLMFGKQGAGRDNVDYSSVMLTSEEEIKTTNPVATEQYGVVADN
LQQANTGPIVGNVNSQGALPGMVWQNRDVYLQGPIWAKIPHTDGN
FHPSPLMGGFGLKHPPPQILIKNTPVPADPPTTFSQAKLASFITQ
YSTGQVSVEIEWELQKENSKRWNPEIQYTSNYYKSTNVDFAVNTE
GTYSEPRPIGTRYLTRNL
wild-type capsid amino acid sequence of AAV11
SEQ ID NO: 41
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEG
AKTAPGKKRPLESPQEPDSSSGIGKKGKQPARKRLNFEEDTGAGD
GPPEGSDTSAMSSDIEMRAAPGGNAVDAGQGSDGVGNASGDWHCD
STWSEGKVTTTSTRTWVLPTYNNHLYLRLGTTSSSNTYNGFSTPW
GYFDFNRFHCHFSPRDWQRLINNNWGLRPKAMRVKIFNIQVKEVT
TSNGETTVANNLTSTVQIFADSSYELPYVMDAGQEGSLPPFPNDV
FMVPQYGYCGIVTGENQNQTDRNAFYCLEYFPSQMLRTGNNFEMA
YNFEKVPFHSMYAHSQSLDRLMNPLLDQYLWHLQSTTSGETLNQG
NAATTFGKIRSGDFAFYRKNWLPGPCVKQQRFSKTASQNYKIPAS
GGNALLKYDTHYTLNNRWSNIAPGPPMATAGPSDGDFSNAQLIFP
GPSVTGNTTTSANNLLFTSEEEIAATNPRDTDMFGQIADNNQNAT
TAPITGNVTAMGVLPGMVWQNRDIYYQGPIWAKIPHADGHFHPSP
LIGGFGLKHPPPQIFIKNTPVPANPATTFTAARVDSFITQYSTGQ
VAVQIEWEIEKERSKRWNPEVQFTSNYGNQSSMLWAPDTTGKYTE
PRVIGSRYLTNHL
wild-type capsid amino acid sequence of AAV12
SEQ ID NO: 42
MAADGYLPDWLEDNLSEGIREWWALKPGAPQPKANQQHQDNGRGL
VLPGYKYLGPFNGLDKGEPVNEADAAALEHDKAYDKQLEQGDNPY
LKYNHADAEFQQRLATDTSFGGNLGRAVFQAKKRILEPLGLVEEG
VKTAPGKKRPLEKTPNRPTNPDSGKAPAKKKQKDGEPADSARRTL
DFEDSGAGDGPPEGSSSGEMSHDAEMRAAPGGNAVEAGQGADGVG
NASGDWHCDSTWSEGRVTTTSTRTWVLPTYNNHLYLRIGTTANSN
TYNGFSTPWGYFDFNRFHCHFSPRDWQRLINNNWGLRPKSMRVKI
FNIQVKEVTTSNGETTVANNLTSTVQIFADSTYELPYVMDAGQEG
SFPPFPNDVFMVPQYGYCGVVTGKNQNQTDRNAFYCLEYFPSQML
RTGNNFEVSYQFEKVPFHSMYAHSQSLDRMMNPLLDQYLWHLQST
TTGNSLNQGTATTTYGKITTGDFAYYRKNWLPGACIKQQKFSKNA
NQNYKIPASGGDALLKYDTHTTLNGRWSNMAPGPPMATAGAGDSD
FSNSQLIFAGPNPSGNTTTSSNNLLFTSEEEIATTNPRDTDMFGQ
IADNNQNATTAPHIANLDAMGIVPGMVWQNRDIYYQGPIWAKVPH
TDGHFHPSPLMGGFGLKHPPPQIFIKNTPVPANPNTTFSAARINS
FLTQYSTGQVAVQIDWEIQKEHSKRWNPEVQFTSNYGTQNSMLWA
PDNAGNYHELRAIGSRFLTHHL
wild-type capsid amino acid sequence of AAV13
SEQ ID NO: 43
MTDGYLPDWLEDNLSEGVREWWALQPGAPKPKANQQHQDNARGLVL
PGYKYLGPGNGLDKGEPVNAADAAALEHDKAYDQQLKAGDNPYLK
YNHADAEFQERLQEDTSFGGNLGRAVFQAKKRILEPLGLVEEAAK
TAPGKKRPVEQSPAEPDSSSGIGKSGQQPARKRLNFGQTGDTESV
PDPQPLGQPPAAPSGVGSTTMASGGGAPMADNNEGADGVGNSSGN
WHCDSQWLGDRVITTSTRTWALPTYNNHLYKQISSQSGATNDNHY
FGYSTPWGYFDFNRFHCHFSPRDWQRLINNNWGFRPKRLNFKLFN
IQVKEVTQNDGTTTIANNLTSTVQVFTDSEYQLPYVLGSAHQGCL
PPFPADVFMVPQYGYLTLNNGSQAVGRSSFYCLEYFPSQMLRTGN
NFQFSYTFEDVPFHSSYAHSQSLDRLMNPLIDQYLYYLNRTQTAS
GTQQSRLLFSQAGPTSMSLQAKNWLPGPCYRQQRLSKQANDNNNS
NFPWTGATKYHLNGRDSLVNPGPAMASHKDDKEKFFPMHGTLIFG
KEGTNANNADLENVMITDEEEIRTTNPVATEQYGTVSNNLQNSNA
GPTTGTVNHQGALPGMVWQDRDVYLQGPIWAKIPHTDGHFHPSPL
MGGFGLKHPPPQIMIKNTPVPANPPTNFSAAKFASFITQYSTGQV
SVEIEWELQKENSKRWNPEIQYTSNYNKSVNVDFTVDTNGVYSEP
RPIGTRYLTRNL
wild-type capsid amino acid sequence of AAVrh10
SEQ ID NO: 44
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEG
AKTAPGKKRPVEPSPQRSPDSSTGIGKKGQQPAKKRLNFGQTGDS
ESVPDPQPIGEPPAGPSGLGSGTMAAGGGAPMADNNEGADGVGSS
SGNWHCDSTWLGDRVITTSTRTWALPTYNNHLYKQISNGTSGGST
NDNTYFGYSTPWGYFDFNRFHCHFSPRDWQRLINNNWGFRPKRLN
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFEFSYQFEDVPFHSSYAHSQSLDRLMNPLIDQYLYYLSR
TQSTGGTAGTQQLLFSQAGPNNMSAQAKNWLPGPCYRQQRVSTTL
SQNNNSNFAWTGATKYHLNGRDSLVNPGVAMATHKDDEERFFPSS
GVLMFGKQGAGKDNVDYSSVMLTSEEEIKTTNPVATEQYGVVADN
LQQQNAAPIVGAVNSQGALPGMVWQNRDVYLQGPIWAKIPHTDGN
FHPSPLMGGFGLKHPPPQILIKNTPVPADPPTTFSQAKLASFITQ
YSTGQVSVEIEWELQKENSKRWNPEIQYTSNYYKSTNVDFAVNTD
GTYSEPRPIGTRYLTRNL
wild-type capsid amino acid sequence of AAVrh74
SEQ ID NO: 45
MAADGYLPDWLEDNLSEGIREWWDLKPGAPKPKANQQKQDNGRGL
VLPGYKYLGPFNGLDKGEPVNAADAAALEHDKAYDQQLQAGDNPY
LRYNHADAEFQERLQEDTSFGGNLGRAVFQAKKRVLEPLGLVESP
VKTAPGKKRPVEPSPQRSPDSSTGIGKKGQQPAKKRLNFGQTGDS
ESVPDPQPIGEPPAGPSGLGSGTMAAGGGAPMADNNEGADGVGSS
SGNWHCDSTWLGDRVITTSTRTWALPTYNNHLYKQISNGTSGGST
NDNTYFGYSTPWGYFDFNRFHCHFSPRDWQRLINNNWGFRPKRLN
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFEFSYNFEDVPFHSSYAHSQSLDRLMNPLIDQYLYYLSR
TQSTGGTAGTQQLLFSQAGPNNMSAQAKNWLPGPCYRQQRVSTTL
SQNNNSNFAWTGATKYHLNGRDSLVNPGVAMATHKDDEERFFPSS
GVLMFGKQGAGKDNVDYSSVMLTSEEEIKTTNPVATEQYGVVADN
LQQQNAAPIVGAVNSQGALPGMVWQNRDVYLQGPIWAKIPHTDGN
FHPSPLMGGFGLKHPPPQILIKNTPVPADPPTTFNQAKLASFITQ
YSTGQVSVEIEWELQKENSKRWNPEIQYTSNYYKSTNVDFAVNTE
GTYSEPRPIGTRYLTRNL

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A variant adeno-associated virus (AAV) capsid polypeptide comprising a peptide insertion in the variable region IV or in the variable region VIII relative to a wild-type AAV capsid polypeptide, wherein the peptide insertion comprises an amino acid sequence selected from the group consisting of SEQ ID NOs: 1-29 or an amino acid sequence having at least 70%, optionally at least 85% sequence identity thereto.

2. The variant polypeptide according to claim 1, wherein the peptide insertion comprises an amino acid sequence selected from the group consisting of SEQ ID NOs: 1-10 or an amino acid sequence having at least 85% sequence identity thereto.

3. The variant polypeptide according to claim 1 or 2, wherein the peptide insertion further comprises a G at the N-terminus and/or an A at the C-terminus.

4. The variant polypeptide according to claim 1, wherein the peptide insertion consists of an amino acid sequence of a G at the N-terminus, followed by an amino acid sequence selected from the group consisting of SEQ ID Nos: 1-10, followed by an A at the C-terminus, or an amino acid sequence having at least 88% sequence identity thereto.

5. The variant polypeptide according to claim 1, wherein the AAV is AAV1 and the peptide insertion is immediately after an amino acid selected from amino acids 580 to 595 (VP1 numbering) of AAV1, wherein the AAV is AAV2 and the peptide insertion is immediately after an amino acid selected from amino acids 579 to 594 (VP1 numbering) of AAV2, wherein the AAV is AAV3 and the peptide insertion is immediately after an amino acid selected from amino acids 580 to 595 (VP1 numbering) of AAV3, wherein the AAV is AAV3b and the peptide insertion is immediately after an amino acid selected from amino acids 580 to 595 (VP1 numbering) of AAV3b, wherein the AAV is AAV4 and the peptide insertion is immediately after an amino acid selected from amino acids 578 to 593 (VP1 numbering) of AAV4, wherein the AAV is AAV5 and the peptide insertion is immediately after an amino acid selected from amino acids 569 to 584 (VP1 numbering) of AAV5, wherein the AAV is AAV6 and the peptide insertion is immediately after an amino acid selected from amino acids 580 to 595 (VP1 numbering) of AAV6, wherein the AAV is AAV7 and the peptide insertion is immediately after an amino acid selected from amino acids 581 to 596 (VP1 numbering) of AAV7, wherein the AAV is AAV8 and the peptide insertion is immediately after an amino acid selected from amino acids 582 to 597 (VP1 numbering) of AAV8, wherein the AAV is AAV9 and the peptide insertion is immediately after an amino acid selected from amino acids 580 to 595 (VP1 numbering) of AAV9, wherein the AAV is AAV10 and the peptide insertion is immediately after an amino acid selected from amino acids 582 to 597 (VP1 numbering) of AAV10, wherein the AAV is AAV11 and the peptide insertion is immediately after an amino acid selected from amino acids 575 to 593 (VP1 numbering) of AAV11, wherein the AAV is AAV12 and the peptide insertion is immediately after an amino acid selected from amino acids 584 to 600 (VP1 numbering) of AAV12, wherein the AAV is AAV13 and the peptide insertion is immediately after an amino acid selected from amino acids 575 to 593 (VP1 numbering) of AAV13, wherein the AAV is AAVrh10 and the peptide insertion is immediately after an amino acid selected from amino acids 582 to 597 (VP1 numbering) of AAV10rh10, or wherein the AAV is AAVrh74 and the peptide insertion is immediately after an amino acid selected from amino acids 582 to 597 (VP1 numbering) of AAVrh74.

6. The variant polypeptide according to claim 1, wherein the AAV is AAV1 and the AAV1 capsid polypeptide excluding the peptide insertion comprises or consists of the wild-type capsid amino acid sequence of AAV1 of SEQ ID NO:30 or an amino acid sequence having at least 80% sequence identity thereto, (b) the AAV is AAV2 and the AAV2 capsid polypeptide excluding the peptide insertion comprises or consists of the wild-type capsid amino acid sequence of AAV2 of SEQ ID NO:31 or an amino acid sequence having at least 80% sequence identity thereto, (c) the AAV is AAV3 and the AAV3 capsid polypeptide excluding the peptide insertion comprises or consists of the wild-type capsid amino acid sequence of AAV3 of SEQ ID NO:32 or an amino acid sequence having at least 80% sequence identity thereto, (d) the AAV is AAV3b and the AAV3b capsid polypeptide excluding the peptide insertion comprises or consists of the wild-type capsid amino acid sequence of AAV3b of SEQ ID NO:33 or an amino acid sequence having at least 80% sequence identity thereto, (e) the AAV is AAV4 and the AAV4 capsid polypeptide excluding the peptide insertion comprises or consists of the wild-type capsid amino acid sequence of AAV4 of SEQ ID NO:34 or an amino acid sequence having at least 80% sequence identity thereto, (f) the AAV is AAV5 and the AAV5 capsid polypeptide excluding the peptide insertion comprises or consists of the wild-type capsid amino acid sequence of AAV5 of SEQ ID NO:35 or an amino acid sequence having at least 80% sequence identity thereto, (g) the AAV is AAV6 and the AAV6 capsid polypeptide excluding the peptide insertion comprises or consists of the wild-type capsid amino acid sequence of AAV6 of SEQ ID NO:36 or an amino acid sequence having at least 80% sequence identity thereto, (h) the AAV is AAV7 and the AAV7 capsid polypeptide excluding the peptide insertion comprises or consists of the wild-type capsid amino acid sequence of AAV7 of SEQ ID NO:37 or an amino acid sequence having at least 80% sequence identity thereto, (i) the AAV is AAV8 and the AAV8 capsid polypeptide excluding the peptide insertion comprises or consists of the wild-type capsid amino acid sequence of AAV8 of SEQ ID NO:38 or an amino acid sequence having at least 80% sequence identity thereto, (j) the AAV is AAV9 and the AAV9 capsid polypeptide excluding the peptide insertion comprises or consists of the wild-type capsid amino acid sequence of AAV9 of SEQ ID NO:39 or an amino acid sequence having at least 80% sequence identity thereto, (k) the AAV is AAV10 and the AAV10 capsid polypeptide excluding the peptide insertion comprises or consists of the wild-type capsid amino acid sequence of AAV10 of SEQ ID NO:40 or an amino acid sequence having at least 80% sequence identity thereto, (I) the AAV is AAV11 and the AAV11 capsid polypeptide excluding the peptide insertion comprises or consists of the wild-type capsid amino acid sequence of AAV11 of SEQ ID NO:41 or an amino acid sequence having at least 80% sequence identity thereto, (m) the AAV is AAV12 and the AAV12 capsid polypeptide excluding the peptide insertion comprises or consists of the wild-type capsid amino acid sequence of AAV12 of SEQ ID NO:42 or an amino acid sequence having at least 80% sequence identity thereto, (n) the AAV is AAV13 and the AAV13 capsid polypeptide excluding the peptide insertion comprises or consists of the wild-type capsid amino acid sequence of AAV13 of SEQ ID NO:43 or an amino acid sequence having at least 80% sequence identity thereto, (o) the AAV is AAVrh10 and the AAVrh10 capsid polypeptide excluding the peptide insertion comprises or consists of the wild-type capsid amino acid sequence of AAVrh10 of SEQ ID NO: 44 or an amino acid sequence having at least 80% sequence identity thereto, or (p) the AAV is AAVrh74 and the AAVrh74 capsid polypeptide excluding the peptide insertion comprises or consists of the wild-type capsid amino acid sequence of AAVrh74 of SEQ ID NO:45 or an amino acid sequence having at least 80% sequence identity thereto.

7. An isolated nucleic acid encoding the variant polypeptide according to claim 1.

8. A recombinant polynucleotide comprising the nucleic acid according to claim 7.

9. An isolated cell comprising the polypeptide of claim 1.

10. An adeno-associated virus (AAV) vector comprising the variant polypeptide according to claim 1, wherein the AAV vector optionally further comprises a heterologous nucleic acid.

11. The AAV vector of claim 10, wherein the heterologous nucleic acid comprises a nucleotide sequence encoding (i) a polypeptide selected from the group consisting of tissue inhibitor of metalloproteinases-3 (TIMP-3), an opsin, an anti-apoptotic polypeptide, fibroblast growth factor 2, epidermal growth factor, Sonic hedgehog, X-linked retinis pigmentosa GTPase regulator (RGPR), retinitis pigmentosa GTPase regulator (RGPR)-interacting protein-1, peripherin-2 (Prph2), MYO7A, Rab escort protein 1 (REP1), lebercilin, retinoschisin, Cyclic Nucleotide Gated Channel Subunit Beta 3 (CNGB3), Cyclic Nucleotide Gated Channel Subunit Alpha 3 (CNGA3), a VDM2 protein, isomerase converting all trans retinol ester to 11-cis-retinol (RPE65), phosphodiesterase 6A (PDE6A), phosphodiesterase 6B (PDE6B), phosphodiesterase 6C (PDE6C), retinaldehyde binding protein 1 (RLBP1), nuclear receptor subfamily 2 group E member 3 (NR2E3), MER proto-oncogene tyrosine kinase (MERTK), NADH dehydrogenase subunit 4 (ND4), Raab escort protein-1 (REP1), RP guanosine triphosphatase regulator (RPGR), complement factor 1 (CF1), Crumbs homolog 1 (CRB1), G-protein subunit alpha transducing 2 (GNAT2), soluble FMS-like tyrosine kinase 1 (sFLT-1), a neuroprotective polypeptide, an angiogenic polypeptide and a site-specific nuclease; (ii) an interfering RNA, (iii) a CRISPR-Cas protein or (iv) an aptamer.

12. A pharmaceutical composition comprising the AAV vector according to claim 10 and a pharmaceutically acceptable carrier and/or excipient.

13. A method of preventing or treating an ocular disease in a subject in need thereof, the method comprising administering to the subject the AAV vector according to claim 10, wherein the ocular disease is optionally selected from the group consisting of glaucoma, retinitis pigmentosa, macular degeneration including age-related macular degeneration, Leber congenital amaurosis, Leber congenital amaurosis type 10, Usher syndrome 2A, diabetic retinopathy, achromatosis, diabetic macular edema, choroideremia, Leber hereditary optic neuropathy, retinoschisis including X-linked juvenile retinoschisis and color blindness.

14. A method of delivering a heterologous nucleic acid to the eye of a subject, the method comprising administering to the subject the AAV vector according to claim 10.

15. The method according to claim 14, wherein the delivering is directed to a retinal cell or a retinal layer in the eye of the subject.

16. A method for enhancing gene transduction in a retina cell or a retina layer, the method comprising administering to the subject the AAV vector according to claim 10, wherein the variant AAV capsid polypeptide comprises a peptide insertion comprising an amino acid sequence selected from the group consisting of SEQ ID NOs: 01 to 10 or an amino acid sequence having at least 70%, optionally at least 85% sequence identity thereto.

17. The method of claim 16, wherein the AAV is AAV2 and the peptide insertion is immediately after an amino acid selected from amino acids 579 to 594 (VP1 numbering) of AAV2 or wherein the AAV is AAV8 and the peptide insertion is immediately after an amino acid selected from amino acids 582 to 597 (VP1 numbering) of AAV8.

18. The method of claim 16, wherein the retina layer is retinal pigment epithelium (RPE).

19. The method of claim 16, wherein the retina cell selected from the group consisting of bipolar cells, RGC, Mueller Glia cells, Amacrine cells, rod cells, microglia cells, horizontal cells, cone cells and vascular cells.
